SEQUENCE LISTING (126 sequences)

SEQ ID NO: 1 - DNA, 261 bp, Chlamydia trachomatis
atgagtcaaaataagaactctgctttcatgcagcctgtgaacgtatccgctgatttagctgccatcgttggtgcaggacctatgcctcgcacagagatcattaagaaaatgtgggattacattaaggagaatagtcttcaagatcctacaaacaaacgtaatatcaatcccgatgataaattggctaaagtttttggaactgaaaaacctatcgatatgttccaaatgacaaaaatggtttctcaacacatcattaaataa

SEQ ID NO: 2 - protein, 86 aa, Chlamydia trachomatis
MSQNKNSAFMQPVNVSADLAAIVGAGPMPRTEIIKKMWDYIKENSLQDPTNKRNINPDDKLAKVFGTEKPIDMFQMTKMVSQHIIK

SEQ ID NO: 3 - DNA, 1122 bp, Chlamydia trachomatis
ctgcctgtggggaatcctgctgaaccaagccttatgatcgacggaattctgtgggaaggtttcggcggagatccttgcgatccttgcaccacttggtgtgacgctatcagcatgcgtatgggttactatggtgactttgttttcgaccgtgttttgaaaacagatgtgaataaagagtttgaaatgggcgaggctttagccggagcttctgggaatacgacctctactctttcaaaattggtagaacgaacgaaccctgcatatggcaagcatatgcaagacgcagagatgtttaccaatgccgcttgcatgacattgaatatttgggatcgttttgatgtattctgtacattaggagccaccagtggatatcttaaaggaaattcagcatctttcaacttagttgggttattcggcgatggtgtaaacgccacgaaacctgctgcagatagtattcctaacgtgcagttaaatcagtctgtggtggaactgtatacagatactacttttgcttggagtgttggagctcgtgcagctttgtgggaatgtggatgtgcaactttaggagcttctttccaatatgctcaatctaaacctaaaatcgaagaattaaacgttctctgtaacgcagcagagtttactattaataaacctaaagggtatgtaggtaaggagtttcctcttgatcttacagcaggaacagatgcagcgacgggcactaaagatgcctctattgattaccatgagtggcaagcaagtttatctctttcttacagactcaatatgttcactccctacattggagttaaatggtctcgtgcaagctttgattctgatacaattcgtatagcccagccgaggttggtaacacctgttgtagatattacaacccttaacccaactattgcaggatgcggcagtgtagctggagctaacacggaaggacagatatctgatacaatgcaaatcgtctccttgcaattgaacaagatgaaatctagaaaatcttgcggtattgcagtaggaacaactattgtggatgcagacaaatacgcagttacagttgagactcgcttgatcgatgagagagctgctcacgtaaatgcacaattccgcttctag

SEQ ID NO: 4 - protein, 373 aa, Chlamydia trachomatis
LPVGNPAEPSLMIDGILWEGFGGDPCDPCTTWCDAISMRMGYYGDFVFDRVLKTDVNKEFEMGEALAGASGNTTSTLSKLVERTNPAYGKHMQDAEMFTNAACMTLNIWDRFDVFCTLGATSGYLKGNSASFNLVGLFGDGVNATKPAADSIPNVQLNQSVVELYTDTTFAWSVGARAALWECGCATLGASFQYAQSKPKIEELNVLCNAAEFTINKPKGYVGKEFPLDLTAGTDAATGTKDASIDYHEWQASLSLSYRLNMFTPYIGVKWSRASFDSDTIRIAQPRLVTPVVDITTLNPTIAGCGSVAGANTEGQISDTMQIVSLQLNKMKSRKSCGIAVGTTIVDADKYAVTVETRLIDERAAHVNAQFRF

SEQ ID NO: 5 - DNA, 1746 bp, Chlamydia trachomatis
gtacgaggagaaagcttggtttgcaagaatgctcttcaagatttgagttttttagagcatttattacaggttaaatatgctcctaaaacatggaaagagcaatacttaggatgggatcttgttcaaagctccgtttctgcacagcagaagcttcgtacacaagaaaatccatcaacaagtttttgccagcaggtccttgctgattttatcggaggattaaatgactttcacgctggagtaactttctttgcgatagaaagtgcttaccttccttataccgtacaaaaaagtagtgacggccgtttctactttgtagatatcatgactttttcttcagagatccgtgttggagatgagttgctagaggtggatggggcgcctgtccaagatgtgctcgctactctatatggaagcaatcacaaagggactgcagctgaagagtcggctgctttaagaacactattttctcgcatggcctctttagggcacaaagtaccttctgggcgcactactttaaagattcgtcgtccttttggtactacgagagaagttcgtgtgaaatggcgttatgttcctgaaggtgtaggagatttggctaccatagctccttctatcagggctccacagttacagaaatcgatgagaagctttttccctaagaaagatgatgcgtttcatcggtctagttcgctattctactctccaatggttccgcatttttgggcagagcttcgcaatcattatgcaacgagtggtttgaaaagcgggtacaatattgggagtaccgatgggtttctccctgtcattgggcctgttatatgggagtcggagggtcttttccgcgcttatatttcttcggtgactgatggggatggtaagagccataaagtaggatttctaagaattcctacatatagttggcaggacatggaagattttgatccttcaggaccgcctccttgggaagaatttgctaagattattcaagtattttcttctaatacagaagctttgattatcgaccaaacgaacaacccaggtggtagtgtcctttatctttatgcactgctttccatgttgacagaccgtcctttagaacttcctaaacatagaatgattctgactcaggatgaagtggttgatgctttagattggttaaccctgttggaaaacgtagacacaaacgtggagtctcgccttgctctgggagacaacatggaaggatatactgtggatctacaggttgccgagtatttaaaaagctttggacgtcaagtattgaattgttggagtaaaggggatatcgagttatcaacacctattcctctttttggttttgagaagattcatccacatcctcgagttcaatactctaaaccgatttgtgttttgatcaatgagcaagacttttcttgtgctgacttcttccctgtagttttgaaagacaatgatcgagctcttattgttggtactcgaacagctggagctggaggatttgtctttaatgtgcagttcccaaatagaactggaataaaaacttgttctttaacaggatcattagctgttagagagcatggtgccttcattgagaacatcggagtcgaaccgcatatcgatctgccttttacagcgaatgatattcgctataaaggctattccgagtatcttgataaggtcaaaaaattggtttgtcagctgatcaataacgacggtaccattattcttgcggaagatggtagtttttaa

SEQ ID NO: 6 - protein, 581 aa, Chlamydia trachomatis
VRGESLVCKNALQDLSFLEHLLQVKYAPKTWKEQYLGWDLVQSSVSAQQKLRTQENPSTSFCQQVLADFIGGLNDFHAGVTFFAIESAYLPYTVQKSSDGRFYFVDIMTFSSEIRVGDELLEVDGAPVQDVLATLYGSNHKGTAAEESAALRTLFSRMASLGHKVPSGRTTLKIRRPFGTTREVRVKWRYVPEGVGDLATIAPSIRAPQLQKSMRSFFPKKDDAFHRSSSLFYSPMVPHFWAELRNHYATSGLKSGYNIGSTDGFLPVIGPVIWESEGLFRAYISSVTDGDGKSHKVGFLRIPTYSWQDMEDFDPSGPPPWEEFAKIIQVFSSNTEALIIDQTNNPGGSVLYLYALLSMLTDRPLELPKHRMILTQDEVVDALDWLTLLENVDTNVESRLALGDNMEGYTVDLQVAEYLKSFGRQVLNCWSKGDIELSTPIPLFGFEKIHPHPRVQYSKPICVLINEQDFSCADFFPVVLKDNDRALIVGTRTAGAGGFVFNVQFPNRTGIKTCSLTGSLAVREHGAFIENIGVEPHIDLPFTANDIRYKGYSEYLDKVKKLVCQLINNDGTIILAEDGSF

SEQ ID NO: 7 - DNA, 1776 bp, Chlamydia trachomatis
atgagcatcaggggagtaggaggcaacgggaatagtcgaatcccttctcataatggggatggatcgaatcgcagaagtcaaaatacgaagggtaataataaagttgaagatcgagtttgttctctatattcatctcgtagtaacgaaaatagagaatctccttatgcagtagtagacgtcagctctatgatcgagagcaccccaacgagtggagagacgacaagagcttcgcgtggagtgctcagtcgtttccaaagaggtttagtacgaatagctgacaaagtaagacgagctgttcagtgtgcgtggagttcagtctctacaagcagatcgtctgcaacaagagccgcagaatccggatcaagtagtcgtactgctcgtggtgcaagttctgggtatagggagtattctccttcagcagctagagggctgcgtcttatgttcacagatttctggagaactcgggttttacgccagacctctcctatggctggagtttttgggaatcttgatgtgaacgaggctcgtttgatggctgcgtacacaagtgagtgcgcggatcatttagaagcgaaggagttggctggccctgacggggtagcggccgcccgggaaattgctaaaagatgggagaaaagagttagagatctacaagataaaggtgctgcacgaaaattattaaatgatcctttaggccgacgaacacctaattatcagagcaaaaatccaggtgagtatactgtagggaattccatgttttacgatggtcctcaggtagcgaatctccagaacgtcgacactggtttttggctggacatgagcaatctctcagacgttgtattatccagagagattcaaacaggacttcgagcacgagctactttggaagaatccatgccgatgttagagaatttagaagagcgttttagacgtttgcaagaaacttgtgatgcggctcgtactgagatagaagaatcgggatggactcgagagtccgcatcaagaatggaaggcgatgaggcgcaaggaccttctagagtacaacaagcttttcagagctttgtaaatgaatgtaacagcatcgagttctcatttgggagctttggagagcatgtgcgagttctctgcgctagagtatcacgaggattagctgccgcaggagaggcgattcgccgttgcttctcttgttgtaaaggatcgacgcatcgctacgctcctcgcgatgacctatctcctgaaggtgcatcgttagcagagactttggctagattcgcagatgatatgggaatagagcgaggtgctgatggaacctacgatattcctttggtagatgattggagaagaggggttcctagtattgaaggagaaggatctgactcgatctatgaaatcatgatgcctatctatgaagttatgaatatggatctagaaacacgaagatcttttgcggtacagcaagggcactatcaggacccaagagcttcagattatgacctcccacgtgctagcgactatgatttgcctagaagcccatatcctactccacctttgcctcctagatatcagctacagaatatggatgtagaagcagggttccgtgaggcagtttatgcttcttttgtagcaggaatgtacaattatgtagtgacacagccgcaagagcgtattcccaatagtcagcaggtggaagggattctgcgtgatatgcttaccaacgggtcacagacatttagagacctgatgaagcgttggaatagagaagtcgatagggaataa

SEQ ID NO: 8 - protein, 591 aa, Chlamydia trachomatis
MSIRGVGGNGNSRIPSHNGDGSNRRSQNTKGNNKVEDRVCSLYSSRSNENRESPYAVVDVSSMIESTPTSGETTRASRGVLSRFQRGLVRIADKVRRAVQCAWSSVSTSRSSATRAAESGSSSRTARGASSGYREYSPSAARGLRLMFTDFWRTRVLRQTSPMAGVFGNLDVNEARLMAAYTSECADHLEAKELAGPDGVAAAREIAKRWEKRVRDLQDKGAARKLLNDPLGRRTPNYQSKNPGEYTVGNSMFYDGPQVANLQNVDTGFWLDMSNLSDVVLSREIQTGLRARATLEESMPMLENLEERFRRLQETCDAARTEIEESGWTRESASRMEGDEAQGPSRVQQAFQSFVNECNSIEFSFGSFGEHVRVLCARVSRGLAAAGEAIRRCFSCCKGSTHRYAPRDDLSPEGASLAETLARFADDMGIERGADGTYDIPLVDDWRRGVPSIEGEGSDSIYEIMMPIYEVMNMDLETRRSFAVQQGHYQDPRASDYDLPRASDYDLPRSPYPTPPLPPRYQLQNMDVEAGFREAVYASFVAGMYNYVVTQPQERIPNSQQVEGILRDMLTNGSQTFRDLMKRWNREVDRE

SEQ ID NO: 9 - DNA, 1962 bp, Chlamydia trachomatis
atggaatcaggaccagaatcagtttcttctaatcagagctcgatgaatccaattattaatgggcaaatcgcttctaattcggagaccaaagagtccacgaaggcgtccgaagcgagtccttcagcatcgtcctctgtaagcagctggagttttttatcctcagcaaagaatgcattaatctctcttcgtgatgccatcttgaataaaaattccagtccaacagactctctctctcaattagaggcctctacttctacctctacggttacacgtgtagcggcaaaagattatgatgaggctaaatcgaattttgatacggcgaaaagtggattagagaacgctaagacacttgctgaatacgaaacgaaaatggctgatttgatggcagctctccaagatatggagcgtttagctaattcagatcctagtaacaatcataccgaagaagtaaataatattaagaaagcgctcgaagcacaaaaagatactattgataagctgaataaactcgttacgctgcaaaatcagaataaatctttaacagaagtgttgaaaacaactgactctgcagatcagattccagcgattaatagtcagttagagatcaacaaaaattctgcagatcaaattatcaaagatctggaaagacaaaacataagttatgaagctgttctcactaacgcaggagaggttatcaaagcttcttctgaagcgggaattaagttaggacaagctttgcagtctattgtggatgctggggaccaaagtcaggctgcagttctgcaagcacagcaaaataatagcccagataatattgcagccacgaaggaattaattgatgctgctgaaacgaaggtaaacgagttaaaacaagagcatacagggctaacggactcgcctttagtgaaaaaagctgaggagcagattagtcaagcacaaaaagatattcaagagatcaaacctagtggttcggatattcctatcgttggtccgagtgggtcagctgcttccgcaggaagtgcggcaggagcgttgaaatcctctaacaattcaggaagaatttccttgttgcttgatgatgtagacaatgaaatggcagcgattgcactgcaaggttttcgatctatgatcgaacaatttaatgtaaacaatcctgcaacagctaaagagctacaagctatggaggctcagctgactgcgatgtcagatcaactggttggtgcggatggcgagctcccagccgaaatacaagcaatcaaagatgctcttgcgcaagctttgaaacaaccatcagcagatggtttggctacagctatgggacaagtggcttttgcagctgccaaggttggaggaggctccgcaggaacagctggcactgtccagatgaatgtaaaacagctttacaagacagcgttttcttcgacttcttccagctcttatgcagcagcactttccgatggatattctgcttacaaaacactgaactctttatattccgaaagcagaagcggcgtgcagtcagctattagtcaaactgcaaatcccgcgctttccagaagcgtttctcgttctggcatagaaagtcaaggacgcagtgcagatgctagccaaagagcagcagaaactattgtcagagatagccaaacgttaggtgatgtatatagccgcttacaggttctggattctttgatgtctacgattgtgagcaatccgcaagcaaatcaagaagagattatgcagaagctcacggcatctattagcaaagctccacaatttgggtatcctgctgttcagaattctgcggatagcttgcagaagtttgctgcgcaattggaaagagagtttgttgatggggaacgtagtctcgcagaatctcaagagaatgcgtttagaaaacagcccgctttcattcaacaggtgttggtaaacattgcttctctattctctggttatctttcttaa

SEQ ID NO: 10 - protein, 653 aa, Chlamydia trachomatis
MESGPESVSSNQSSMNPIINGQIASNSETKESTKASEASPSASSSVSSWSFLSSAKNALISLRDAILNKNSSPTDSLSQLEASTSTSTVTRVAAKDYDEAKSNFDTAKSGLENAKTLAEYETKMADLMAALQDMERLANSDPSNNHTEEVNNIKKALEAQKDTIDKLNKLVTLQNQNKSLTEVLKTTDSADQIPAINSQLEINKNSADQIIKDLERQNISYEAVLTNAGEVIKASSEAGIKLGQALQSIVDAGDQSQAAVLQAQQNNSPDNIAATKELIDAAETKVNELKQEHTGLTDSPLVKKAEEQISQAQKDIQEIKPSGSDIPIVGPSGSAASAGSAAGALKSSNNSGRISLLLDDVDNEMAAIALQGFRSMIEQFNVNNPATAKELQAMEAQLTAMSDQLVGADGELPAEIQAIKDALAQALKQPSADGLATAMGQVAFAAAKVGGGSAGTAGTVQMNVKQLYKTAFSSTSSSSYAAALSDGYSAYKTLNSLYSESRSGVQSAISQTANPALSRSVSRSGIESQGRSADASQRAAETIVRDSQTLGDVYSRLQVLDSLMSTIVSNPQANQEEIMQKLTASISKAPQFGYPAVQNSADSLQKFAAQLEREFVDGERSLAESQENAFRKQPAFIQQVLVNIASLFSGYLS

SEQ ID NO: 11 - DNA, 2010 bp, Chlamydia trachomatis
gcagaaatcatgattcctcaaggaatttacgatggggagacgttaactgtatcatttccctatactgttataggagatccgagtgggactactgttttttctgcaggagagttaacattaaaaaatcttgacaattctattgcagctttgcctttaagttgttttgggaacttattagggagttttactgttttagggagaggacactcgttgactttcgagaacatacggacttctacaaatggggcagctctaagtaatagcgctgctgatggactgtttactattgagggttttaaagaattatccttttccaattgcaattcattacttgccgtactgcctgctgcaacgactaataagggtagccagactccgacgacaacatctacaccgtctaatggtactatttattctaaaacagatcttttgttactcaataatgagaagttctcattctatagtaatttagtctctggagatgggggagctatagatgctaagagcttaacggttcaaggaattagcaagctttgtgtcttccaagaaaatactgctcaagctgatgggggagcttgtcaagtagtcaccagtttctctgctatggctaacgaggctcctattgcctttgtagcgaatgttgcaggagtaagagggggagggattgctgctgttcaggatgggcagcagggagtgtcatcatctacttcaacagaagatccagtagtaagtttttccagaaatactgcggtagagtttgatgggaacgtagcccgagtaggaggagggatttactcctacgggaacgttgctttcctgaataatggaaaaaccttgtttctcaacaatgttgcttctcctgtttacattgctgctaagcaaccaacaagtggacaggcttctaatacgagtaataattacggagatggaggagctatcttctgtaagaatggtgcgcaagcaggatccaataactctggatcagtttcctttgatggagagggagtagttttctttagtagcaatgtagctgctgggaaagggggagctatttatgccaaaaagctctcggttgctaactgtggccctgtacaatttttaaggaatatcgctaatgatggtggagcgatttatttaggagaatctggagagctcagtttatctgctgattatggagatattattttcgatgggaatcttaaaagaacagccaaagagaatgctgccgatgttaatggcgtaactgtgtcctcacaagccatttcgatgggatcgggagggaaaataacgacattaagagctaaagcagggcatcagattctctttaatgatcccatcgagatggcaaacggaaataaccagccagcgcagtcttccaaacttctaaaaattaacgatggtgaaggatacacaggggatattgtttttgctaatggaagcagtactttgtaccaaaatgttacgatagagcaaggaaggattgttcttcgtgaaaaggcaaaattatcagtgaattctctaagtcagacaggtgggagtctgtatatggaagctgggagtacattggattttgtaactccacaaccaccacaacagcctcctgccgctaatcagttgatcacgctttccaatctgcatttgtctctttcttctttgttagcaaacaatgcagttacgaatcctcctaccaatcctccagcgcaagattctcatcctgcagtcattggtagcacaactgctggttctgttacaattagtgggcctatcttttttgaggatttggatgatacagcttatgataggtatgattggctaggttctaatcaaaaaatcaatgtcctgaaattacagttagggactaagcccccagctaatgccccatcagatttgactctagggaatgagatgcctaagtatggctatcaaggaagctggaagcttgcgtgggatcctaatacagcaaataatggtccttatactctgaaagctacatggactaaaactgggtaa

SEQ ID NO: 12 - protein, 669 aa, Chlamydia trachomatis
AEIMIPQGIYDGETLTVSFPYTVIGDPSGTTVFSAGELTLKNLDNSIAALPLSCFGNLLGSFTVLGRGHSLTFENIRTSTNGAALSNSAADGLFTIEGFKELSFSNCNSLLAVLPAATTNKGSQTPTTTSTPSNGTIYSKTDLLLLNNEKFSFYSNLVSGDGGAIDAKSLTVQGISKLCVFQENTAQADGGACQVVTSFSAMANEAPIAFVANVAGVRGGGIAAVQDGQQGVSSSTSTEDPVVSFSRNTAVEFDGNVARVGGGIYSYGNVAFLNNGKTLFLNNVASPVYIAAKQPTSGQASNTSNNYGDGGAIFCKNGAQAGSNNSGSVSFDGEGVVFFSSNVAAGKGGAIYAKKLSVANCGPVQFLRNIANDGGAIYLGESGELSLSADYGDIIFDGNLKRTAKENAADVNGVTVSSQAISMGSGGKITTLRAKAGHQILFNDPIEMANGNNQPAQSSKLLKINDGEGYTGDIVFANGSSTLYQNVTIEQGRIVLREKAKLSVNSLSQTGGSLYMEAGSTLDFVTPQPPQQPPAANQLITLSNLHLSLSSLLANNAVTNPPTNPPAQDSHPAVIGSTTAGSVTISGPIFFEDLDDTAYDRYDWLGSNQKINVLKLQLGTKPPANAPSDLTLGNEMPKYGYQGSWKLAWDPNTANNGPYTLKATWTKTG

SEQ ID NO: 13 - DNA, 3519 bp, Chlamydia trachomatis
agttgcgtagatcttcatgctggaggacagtctgtaaatgagctggtatatgtaggccctcaagcggttttattgttagaccaaattcgagatctattcgttgggtctaaagatagtcaggctgaaggacagtataggttaattgtaggagatccaagttctttccaagagaaagatgcagatactcttcccgggaaggtagagcaaagtactttgttctcagtaaccaatcccgtggttttccaaggtgtggaccaacaggatcaagtctcttcccaagggttaatttgtagttttacgagcagcaaccttgattctccccgtgacggagaatcttttttaggtattgcttttgttggggatagtagtaaggctggaatcacattaactgacgtgaaagcttctttgtctggagcggctttatattctacagaagatcttatctttgaaaagattaagggtggattggaatttgcatcatgttcttctctagaacaggggggagcttgtgcagctcaaagtattttgattcatgattgtcaaggattgcaggttaaacactgtactacagccgtgaatgctgaggggtctagtgcgaatgatcatcttggatttggaggaggcgctttctttgttacgggttctctttctggagagaaaagtctctatatgcctgcaggagatatggtagttgcgaattgtgatggggctatatcttttgaaggaaacagcgcgaactttgctaatggaggagcgattgctgcctctgggaaagtgctttttgtcgctaatgataaaaagacttcttttatagagaaccgagctttgtctggaggagcgattgcagcctcttctgatattgcctttcaaaactgcgcagaactagttttcaaaggcaattgtgcaattggaacagaggataaaggttctttaggtggaggggctatatcttctctaggcaccgttcttttgcaagggaatcacgggataacttgtgataagaatgagtctgcttcgcaaggaggcgccatttttggcaaaaattgtcagatttctgacaacgaggggccagtggttttcagagatagtacagcttgcttaggaggaggcgctattgcagctcaagaaattgtttctattcagaacaatcaggctgggatttccttcgagggaggtaaggctagtttcggaggaggtattgcgtgtggatctttttcttccgcaggcggtgcttctgttttagggactattgatatttcgaagaatttaggcgcgatttcgttctctcgtactttatgtacgacctcagatttaggacaaatggagtaccagggaggaggagctctatttggtgaaaatatttctctttctgagaatgctggtgtgctcacctttaaagacaacattgtgaagacttttgcttcgaatgggaaaattctgggaggaggagcgattttagctactggtaaggtggaaattaccaataattccggaggaatttcttttacaggaaatgcgagagctccacaagctcttccaactcaagaggagtttcctttattcagcaaaaaagaagggcgaccactctcttcaggatattctgggggaggagcgattttaggaagagaagtagctattctccacaacgctgcagtagtatttgagcaaaatcgtttgcagtgcagcgaagaagaagcgacattattaggttgttgtggaggaggcgctgttcatgggatggatagcacttcgattgttggcaactcttcagtaagatttggtaataattacgcaatgggacaaggagtctcaggaggagctcttttatctaaaacagtgcagttagctggaaatggaagcgtcgatttttctcgaaatattgctagtttgggaggaggagctcttcaagcttctgaaggaaattgtgagctagttgataacggctatgtgctattcagagataatcgagggagggtttatgggggtgctatttcttgcttacgtggagatgtagtcatttctggaaacaagggtagagttgaatttaaagacaacatagcaacacgtctttatgtggaagaaactgtagaaaaggttgaagaggtagagccagctcctgagcaaaaagacaataatgagctttctttcttagggagtgtagaacagagttttattactgcagctaatcaagctcttttcgcatctgaagatggggatttatcacctgagtcatccatttcttctgaagaacttgcgaaaagaagagagtgtgctggaggagctatttttgcaaaacgggttcgtattgtagataaccaagaggccgttgtattctcgaataacttctctgatatttatggcggcgccatttttacaggttctcttcgagaagaggataagttagatgggcaaatccctgaagtcttgatctcaggcaatgcaggggatgttgttttttccggaaattcctcgaagcgtgatgagcatcttcctcatacaggtgggggagccatttgtactcaaaatttgacgatttctcagaatacagggaatgttctgttttataacaacgtggcctgttcgggaggagctgttcgtatagaggatcatggtaatgttcttttagaagcttttggaggagatattgtttttaaaggaaattcttctttcagagcacaaggatccgatgctatctattttgcaggtaaagaatcgcatattacagccctgaatgctacggaaggacatgctattgttttccacgacgcattagtttttgaaaatctaaaagaaaggaaatctgctgaagtattgttaatcaatagtcgagaaaatccaggttacactggatctattcgatttttagaagcagaaagtaaagttcctcaatgtattcatgtacaacaaggaagccttgagttgctaaatggagctacattatgtagttatggttttaaacaagatgctggagctaagttggtattggctgctggatctaaactgaagattttagattcaggaactcctgtacaagggcatgctatcagtaaacctgaagcagaaatcgagtcatcttctgaaccagagggtgcacattctctttggattgcgaagaatgctcaaacaacagttcctatggttgatatccatactatttctgtagatttagcctccttctcttctagtcaacaggaggggacagtagaagctcctcaggttattgttcctggaggaagttatgttcgatctggagagcttaatttggagttagttaacacaacaggtactggttatgaaaatcatgctttgttgaagaatgaggctaaagttccattgatgtctttcgttgcttctagtgatgaagcttcagccgaaatcagtaacttgtcggtttctgatttacagattcatgtagcaactccagagattgaagaagacacatacggccatatgggagattggtctgaggctaaaattcaagatggaactcttgtcattagttggaatcctactggataa

SEQ ID NO: 14 - protein, 1172 aa, Chlamydia trachomatis
SCVDLHAGGQSVNELVYVGPQAVLLLDQIRDLFVGSKDSQAEGQYRLIVGDPSSFQEKDADTLPGKVEQSTLFSVTNPVVFQGVDQQDQVSSQGLICSFTSSNLDSPRDGESFLGIAFVGDSSKAGITLTDVKASLSGAALYSTEDLIFEKIKGGLEFASCSSLEQGGACAAQSILIHDCQGLQVKHCTTAVNAEGSSANDHLGFGGGAFFVTGSLSGEKSLYMPAGDMVVANCDGAISFEGNSANFANGGAIAASGKVLFVANDKKTSFIENRALSGGAIAASSDIAFQNCAELVFKGNCAIGTEDKGSLGGGAISSLGTVLLQGNHGITCDKNESASQGGAIFGKNCQISDNEGPVVFRDSTACLGGGAIAAQEIVSIQNNQAGISFEGGKASFGGGIACGSFSSAGGASVLGTIDISKNLGAISFSRTLCTTSDLGQMEYQGGGALFGENISLSENAGVLTFKDNIVKTFASNGKILGGGAILATGKVEITNNSGGISFTGNARAPQALPTQEEFPLFSKKEGRPLSSGYSGGGAILGREVAILHNAAVVFEQNRLQCSEEEATLLGCCGGGAVHGMDSTSIVGNSSVRFGNNYAMGQGVSGGALLSKTVQLAGNGSVDFSRNIASLGGGALQASEGNCELVDNGYVLFRDNRGRVYGGAISCLRGDVVISGNKGRVEFKDNIATRLYVEETVEKVEEVEPAPEQKDNNELSFLGSVEQSFITAANQALFASEDGDLSPESSISSEELAKRRECAGGAIFAKRVRIVDNQEAVVFSNNFSDIYGGAIFTGSLREEDKLDGQIPEVLISGNAGDVVFSGNSSKRDEHLPHTGGGAICTQNLTISQNTGNVLFYNNVACSGGAVRIEDHGNVLLEAFGGDIVFKGNSSFRAQGSDAIYFAGKESHITALNATEGHAIVFHDALVFENLKERKSAEVLLINSRENPGYTGSIRFLEAESKVPQCIHVQQGSLELLNGATLCSYGFKQDAGAKLVLAAGSKLKILDSGTPVQGHAISKPEAEIESSSEPEGAHSLWIAKNAQTTVPMVDIHTISVDLASFSSSQQEGTVEAPQVIVPGGSYVRSGELNLELVNTTGTGYENHALLKNEAKVPLMSFVASSDEASAEISNLSVSDLQIHVATPEIEEDTYGHMGDWSEAKIQDGTLVISWNPTG

SEQ ID NO: 15 - DNA, 1266 bp, Chlamydia trachomatis
atgactgcatcaggaggagctggagggctaggcagcacccaaacagtagacgttgcgcgagcacaagctgctgcagctactcaagatgcacaagaggttatcggctctcaggaagcttctgaggcaagtatgctcaaaggatgtgaggatctcataaatcctgcagctgcaacccgaatcaaaaaaaaagaagagaagtttgaatcattagaagctcgtcgcaaaccaacagcggataaagcagaaaagaaatccgagagcacagaggaaaaaggcgatactcctcttgaagatcgtttcacagaagatctttccgaagtctccggagaagattttcgaggattgaaaaattcgttcgatgatgattcttctcctgaagaaattctcgatgcgctcacaagtaaattttctgatcccacaataaaggatctagctcttgattatctaattcaaacagctccctctgataggaaacttaagtccgctctcattcaggcaaagcatcaactgatgagccagaatcctcaggcgattgttggaggacgcaatgttctgttagcttcagaaacctttgcttccagagcaaatacatctccttcatcgcttcgctccttatatctccaagtaacctcatccccctctaattgtgataatttacgtcaaatgcttgcttcttacttgccatcagagaaaaccgctgttatggagtttctagtaaatggcatggtagcagatttaaaatcggagggcccttccattcctcctgcaaaattgcaagtatatatgacggaactaagcaatctccaagccttacactctgtagatagcttttttgatagaaatattgggaacttggaaaatagcttaaagcatgaaggacatgcccctattccatccttaacgacaggaaatttaactaaaaccttcttacaattagtagaagataaattcccttcctcttccaaagctcaaaaggcattaaatgaactggtaggcccagatactggtcctcaaactgaagttttaaacttattcttccgcgctcttaatggctgttcgcctagaatattctctggagctgaaaaaaaacagcagctggcatcggttatcacaaatacgctagatgcgataaatgcggataatgaggattatcctaaaccaggtgacttcccacgatcttccttctctagtacgcctcctcatgctccagtacctcaatctgagattccaacgtcacctacctcaacacagcctccatcaccctaa

SEQ ID NO: 16 - protein, 421 aa, Chlamydia trachomatis
MTASGGAGGLGSTQTVDVARAQAAAATQDAQEVIGSQEASEASMLKGCEDLINPAAATRIKKKEEKFESLEARRKPTADKAEKKSESTEEKGDTPLEDRFTEDLSEVSGEDFRGLKNSFDDDSSPEEILDALTSKFSDPTIKDLALDYLIQTAPSDRKLKSALIQAKHQLMSQNPQAIVGGRNVLLASETFASRANTSPSSLRSLYLQVTSSPSNCDNLRQMLASYLPSEKTAVMEFLVNGMVADLKSEGPSIPPAKLQVYMTELSNLQALHSVDSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 17 - DNA, 1170 bp, Chlamydia psitacci
atgaaaaaactcttgaaatcggcattattgtttgccactacgggttccgctctctccttacaagccttgcctgtagggaatccagctgaaccaagtttattaattgatggcactatgtgggaaggcgcttcaggcgatccttgtgatccttgctctacttggtgtgatgctatcagcatccgcgcagggtactacggagattatgttttcgatcgcatcttaaaagttgatgttaataaaactatcagcatggggacagctccaactggtaatgcagctgctgactttaaaaccgttgcagacaggaataacatagcctacggcaaacatatgcaagatgcagaatggtccacaaacgcggctttcttagcattaaacatttgggatcgttttgatgtcttctgcacattaggggcatctaacggctatctcaaagcaaatgctgcagctttcaatctagtcggcttacttggggtaacaggaacagatcttcaaggccaatatccaaacgtagccatctctcaaggccttgtagagctttatactgacacaaccttctcttggagcgttggtgcgcgtggagctttatgggaatgtggttgcgcaactttaggagcagagttccaatatgcgcagtctaatcctaagatcgaaatgcttaatgtaatttctagcccaacacaatttgtgattcataagcctagaggatataaagggacagcggccaacttccctctgcctttaaccgctggaacagagagcgctactgatactaaatcagctacaattaagtatcatgaatggcaaattggtttagctctttcttatagattgaacatgcttgttccatatattggagtaaactggtccagagctacatttgatgctgactctatccgcattgctcagcctaaattacctacggccattttaaacctaactacatggaaccctactttattaggggaggctactactataaacactggagcaaaatatgctgaccagttacaaattgcttcgcttcaaatcaacaaaatgaagtctagaaaagcttgtggtattgctgttggtgcaaccttaattgatgctgacaaatggtcgatcactggtgaagctcgcttaatcaacgaaagagctgctcacgtaaacgctcaattcagattctaa

SEQ ID NO: 18 - protein, 389 aa, Chlamydia psitacci
MKKLLKSALLFATTGSALSLQALPVGNPAEPSLLIDGTMWEGASGDPCDPCSTWCDAISIRAGYYGDYVFDRILKVDVNKTISMGTAPTGNAAADFKTVADRNNIAYGKHMQDAEWSTNAAFLALNIWDRFDVFCTLGASNGYLKANAAAFNLVGLLGVTGTDLQGQYPNVAISQGLVELYTDTTFSWSVGARGALWECGCATLGAEFQYAQSNPKIEMLNVISSPTQFVIHKPRGYKGTAANFPLPLTAGTESATDTKSATIKYHEWQIGLALSYRLNMLVPYIGVNWSRATFDADSIRIAQPKLPTAILNLTTWNPTLLGEATTINTGAKYADQLQIASLQINKMKSRKACGIAVGATLIDADKWSITGEARLINERAAHVNAQFRF

SEQ ID NO: 19 - DNA, 1170 bp, Chlamydia pneumoniae
atgaaaaaactcttaaagtcggcgttattatccgccgcatttgctggttctgttggctccttacaagccttgcctgtagggaacccttctgatccaagcttattaattgatggtacaatatgggaaggtgctgcaggagatccttgcgatccttgcgctacttggtgcgacgctattagcttacgtgctggattttacggagactatgttttcgaccgtatcttaaaagtagatgcacctaaaacattttctatgggagccaagcctactggatccgctgctgcaaactatactactgccgtagatagacctaacccggcctacaataagcatttacacgatgcagagtggttcactaatgcaggcttcattgccttaaacatttgggatcgctttgatgttttctgtactttaggagcttctaatggttacattagaggaaactctacagcgttcaatctcgttggtttattcggagttaaaggtactactgtaaatgcaaatgaactaccaaacgtttctttaagtaacggagttgttgaactttacacagacacctctttctcttggagcgtaggcgctcgtggagccttatgggaatgcggttgtgcaactttgggagctgaattccaatatgcacagtccaaacctaaagttgaagaacttaatgtgatctgtaacgtatcgcaattctctgtaaacaaacccaagggctataaaggcgttgctttccccttgccaacagacgctggcgtagcaacagctactggaacaaagtctgcgaccatcaattatcatgaatggcaagtaggagcctctctatcttacagactaaactctttagtgccatacattggagtacaatggtctcgagcaacttttgatgctgataacatccgcattgctcagccaaaactacctacagctgttttaaacttaactgcatggaacccttctttactaggaaatgccacagcattgtctactactgattcgttctcagacttcatgcaaattgtttcctgtcagatcaacaagtttaaatctagaaaagcttgtggagttactgtaggagctactttagttgatgctgataaatggtcacttactgcagaagctcgtttaattaacgagagagctgctcacgtatctggtcagttcagattctaa

SEQ ID NO: 20 - protein, 389 aa, Chlamydia pneumoniae
MKKLLKSALLSAAFAGSVGSLQALPVGNPSDPSLLIDGTIWEGAAGDPCDPCATWCDAISLRAGFYGDYVFDRILKVDAPKTFSMGAKPTGSAAANYTTAVDRPNPAYNKHLHDAEWFTNAGFIALNIWDRFDVFCTLGASNGYIRGNSTAFNLVGLFGVKGTTVNANELPNVSLSNGVVELYTDTSFSWSVGARGALWECGCATLGAEFQYAQSKPKVEELNVICNVSQFSVNKPKGYKGVAFPLPTDAGVATATGTKSATINYHEWQVGASLSYRLNSLVPYIGVQWSRATFDADNIRIAQPKLPTAVLNLTAWNPSLLGNATALSTTDSFSDFMQIVSCQINKFKSRKACGVTVGATLVDADKWSLTAEARLINERAAHVSGQFRF

SEQ ID NO: 21 - DNA, 1776 bp, Chlamydia trachomatis
atgagcatcaggggagtaggaggcaacgggaatagtcgaatcccttctcataatggggatggatcgaatcgcagaagtcaaaatacgaagggtaataataaagttgaagatcgagtttgttctctatattcatctcgtagtaacgaaaatagagaatctccttatgcagtagtagacgtcagctctatgatcgagagcaccccaacgagtggagagacgacaagagcttcgcgtggagtgttcagtcgtttccaaagaggtttagtacgagtagctgacaaagtaagacgagctgttcagtgtgcgtggagttcagtctctacaagaagatcgtctgcaacaagagccgcagaatccggatcaagtagtcgtactgctcgtggtgcaagttctgggtatagggagtattctccttcagcagctagagggctgcgtcttatgttcacagatttctggagaactcgggttttacgccagacctctcctatggctggagtttttgggaatcttgatgtgaacgaggctcgtttgatggctgcgtacacaagtgagtgcgcggatcatttagaagcgaacaagttggctggccctgacggggtagcggccgcccgggaaattgctaaaagatgggagcaaagagttagagatctacaagataaaggtgctgcacgaaaattattaaatgatcctttaggccgacgaacacctaattatcagagcaaaaatccaggtgagtatactgtagggaattccatgttttacgatggtcctcaggtagcgaatctccagaacgtcgacactggtttttggctggacatgagcaatctctcagacgttgtattatccagagagattcaaacaggacttcgagcacgagctactttggaagaatccatgccgatgttagagaatttagaagagcgttttagacgtttgcaagaaacttgtgatgcggctcgtactgagatagaagaatcgggatggactcgagagtccgcatcaagaatggaaggcgatgaggcgcaaggaccttctagagcacaacaagcttttcagagctttgtaaatgaatgtaacagcatcgagttctcatttgggagctttggagagcatgtgcgagttctctgcgctagagtatcacgaggattagctgccgcaggagaggcgattcgccgttgcttctcttgttgtaaaggatcgacgcatcgctacgctcctcgcgatgacctatctcctgaaggtgcatcgttagcagagactttggctagattcgcagatgatatgggaatagagcgaggtgctgatggaacctacgatattcctttggtagatgattggagaagaggggttcctagtattgaaggagaaggatctgactcgatctatgaaatcatgatgcctatctatgaagttatggatatggatctagaaacacgaagatcttttgcggtacagcaagggcactatcaggacccaagagcttcagattatgacctcccacgtgctagcgactatgatttgcctagaagcccatatcctactccacctttgcctcctagatatcagctacagaatatggatgtagaagcagggttccgtgaggcagtttatgcttcttttgtagcaggaatgtacaattatgtagtgacacagccgcaagagcgtattcccaatagtcagcaggtggaagggattctgcgtgatatgcttaccaacgggtcacagacatttagagacctgatgaggcgttggaatagagaagtcgatagggaataa

SEQ ID NO: 22 - protein, 591 aa, Chlamydia trachomatis
MSIRGVGGNGNSRIPSHNGDGSNRRSQNTKGNNKVEDRVCSLYSSRSNENRESPYAVVDVSSMIESTPTSGETTRASRGVFSRFQRGLVRVADKVRRAVQCAWSSVSTRRSSATRAAESGSSSRTARGASSGYREYSPSAARGLRLMFTDFWRTRVLRQTSPMAGVFGNLDVNEARLMAAYTSECADHLEANKLAGPDGVAAAREIAKRWEQRVRDLQDKGAARKLLNDPLGRRTPNYQSKNPGEYTVGNSMFYDGPQVANLQNVDTGFWLDMSNLSDVVLSREIQTGLRARATLEESMPMLENLEERFRRLQETCDAARTEIEESGWTRESASRMEGDEAQGPSRAQQAFQSFVNECNSIEFSFGSFGEHVRVLCARVSRGLAAAGEAIRRCFSCCKGSTHRYAPRDDLSPEGASLAETLARFADDMGIERGADGTYDIPLVDDWRRGVPSIEGEGSDSIYEIMMPIYEVMDMDLETRRSFAVQQGHYQDPRASDYDLPRASDYDLPRSPYPTPPLPPRYQLQNMDVEAGFREAVYASFVAGMYNYVVTQPQERIPNSQQVEGILRDMLTNGSQTFRDLMRRWNREVDRE

SEQ ID NO: 23 - DNA, 1845 bp, Chlamydia muridarum
atgttttattttttaggttggtttgttatgggcatcaagggagtaggcggtagcggtcatagcgattatccaatcccttctcataatggagatggggagagtgaaaaaaacagctcagattcaacaagtagtaaggttaatgcaaaagttacttcttccttacagggggctccgtcaacgaatgatgaaaattcagtttccccttattctgtggtggatgtcactgatttaatagagagcggagagtcttctaggcatgtaataaagaaatctatagaaacagaagaagctgctcatcgagaatctagtgtagagggggctgggcattcttctcgcggaatatttggacggttgcaagcaggattaggacgtctggctagaagagtgggggaagctgtcagaaatactgtaggctctatctttccacaaagagctggtgctgagcaaagaacaggcaaagctcggacaaaatattccccttcagcatcaagaggattacgcctcatgttcacagacttctggcgatatcgagttttgcatcggaatcctcctatggatggactttttgcaaagcttgatgccgatgaggctgaagatatggcagcttacacgaaagagtatgttagcaatctagaaaaacgaggagcagctgatcgagaaactatagaacactgtcaaatggtagctaaaaattgggaaaaaagagctagagatttgcgagacatgggggccgcaaaaaaatttttacgcgacccttttggtaagagtgatcctaagtataaggggacactgcctggagaatacactgtcggaaataccatgttttacgatggaccaggtgtgagcaaactatcagaggttgatacaggtttttggttggacatggagaagctctcggatgctgtcttgtctgcaaatattcaaaaagggcttcgagctcgatttgttttaaatcagtctattccacagttagagagtctagaagagcgttttagaaaactggagagtgcttgcgatgaggctcgtgcttcgttaaaggaagcaggttggataaaagaaggcaaggaacctaacaaagcgcaacgagcttttcggcgatttgtagaagaaagccggaatctagagctttcttttggtagttttggagaaagtgctcgtcgtctttccgctcgtgtttcccaaggtttagctgctgcaggggaggcaattcgccgctgctttgattgtcgcaaaggcaaatattcccttaaaaaggacttgtcttctgaagaattaaatttggcagaagagttaattaggtttactgatgagatggggatagagagagacccagatggaaattacaatattccttgggtagaaaactggagaacaggagttcctgttattgaaggagaaggggcagaacatatttatgaaacgatgatgcctgtccaggaatcttttgagcaggtttatgaagttatggatatgggattggaagagcgtagggattttgctgtgagtcaacaacactatcaagttcctcctagatcttcgttgaattacgagactccgcgattcagagaatatgacgttccacgtaattccgctcgttcttattacgatgttccaagagtacctccccaaaatgaggtagaagagatgcatgtgactaaaggaatgaggagttctgtgtatgcttgttttgtagcaggaatgcgcaactacattgtttcacagccacaagaacagattccaaattctgaacaggtggagcagcttttccaagagcttattaacgatggggatcagataattcaagagcttatgaagatatggaatgaggaactagataatcaataa

SEQ ID NO: 24 - protein, 614 aa, Chlamydia muridarum
MFYFLGWFVMGIKGVGGSGHSDYPIPSHNGDGESEKNSSDSTSSKVNAKVTSSLQGAPSTNDENSVSPYSVVDVTDLIESGESSRHVIKKSIETEEAAHRESSVEGAGHSSRGIFGRLQAGLGRLARRVGEAVRNTVGSIFPQRAGAEQRTGKARTKYSPSASRGLRLMFTDFWRYRVLHRNPPMDGLFAKLDADEAEDMAAYTKEYVSNLEKRGAADRETIEHCQMVAKNWEKRARDLRDMGAAKKFLRDPFGKSDPKYKGTLPGEYTVGNTMFYDGPGVSKLSEVDTGFWLDMEKLSDAVLSANIQKGLRARFVLNQSIPQLESLEERFRKLESACDEARASLKEAGWIKEGKEPNKAQRAFRRFVEESRNLELSFGSFGESARRLSARVSQGLAAAGEAIRRCFDCRKGKYSLKKDLSSEELNLAEELIRFTDEMGIERDPDGNYNIPWVENWRTGVPVIEGEGAEHIYETMMPVQESFEQVYEVMDMGLEERRDFAVSQQHYQVPPRSSLNYETPRFREYDVPRNSARSYYDVPRVPPQNEVEEMHVTKGMRSSVYACFVAGMRNYIVSQPQEQIPNSEQVEQLFQELINDGDQIIQELMKIWNEELDNQ

SEQ ID NO: 25 - DNA, 2346 bp, Chlamydia psitacci
atggctggggtaagcggaattggaggtggcggtgggccagggaaactccctcctcatggaaatgatgatgataaacaatctaaatcagccagtttcggaggccatgatatagtttttggagatggggagcgctctagatccggtagtgtgagtagtgaacactcaatagaggagagaacccggacgttaatggaggagggttttcaagtacgcactcctgaagaggtagaggaaactcgaagagcgtctatttccccagaggaagcatctaacccaggattcttttctcgtatatggtcatctgttaagggaatattcacaggtgggaaaaagagtgatagagctcaaggaccagaaatttcctctcctatcattgcgggatataaacgtcatggcgtgcgtcttcctgatgcgcgcgctatgcaggcacatttgcaaagtcaaagtctccaagagatttctgcatcagacgtttcagaaataggagatttagattctggggatacagatatcacggatatttctgatgaaagttcgctacagtcgatagatttggatacagacgatagagccgaagcttctacatcttcagggagaggtgttggtggattggcggctcgtgttcgtggtttgtgggattttgctactaggcagcaagaaactcctgttgatggatttacggggatgactttttctgagttggtcgatacggtccaattgtatgatcagatgattttagatgcggacaatgagactgagcggcaggaactcttaaagtatcgcgatatgtatcaaagctatgttaatacgatgttaggtgagggcaatacctcacctacagatcagttcgatgtgagtgcttctgctggtatcccaggggcttcttctagaagatatagcgatggcgttggagaagcgagatttttagacatagatgacgatttatccagtgtgtcggaaagtgagcttttagatgctatagaaagtggagagtatgccgatcatgtcttagaagagatcagccctgaagtaagaagagttttagatgaagctaataacttgcgtttacagtttgatatggaagtttctgcaagtgtaacaccttcattaagagagcgtattcaatttgctcttgtgaggttggaaagagggattatccgtatacttactttgattagacgtaacctagtcgctctagcacgtttagtaagaagaggtcttcgatcccttggggagcttgtaagacgttgttgcgtgcgtgagagaggtgtttacagatttcttggtagagatcgggcttatgctagggaggccgaaagatttattcaaaggcataccaactcagagaatttttacagtccaggaactcttacggttccctatgaggttgtaaacgcttgggtaaatggaagacctgatgttgtctatgtttctgatgttagaggtatgtttggtcatgaagttgtgagacttcacgttgatgatcgtgagggtacatatgagataattggctctagctggattccttatgaaagtgatggtggggatacacccccacctttaccgggaaatcatcctagtttagattacgcagatattaacgatgactctgaagatcttcccacaacaggggatagggatgctgagccgctatatgctcagatgagaccccgccctcgtggaagagatgagggaccgatttacgatgtcccaagtcctcaaagtagaaggcccagagcaggtgatgatagggatacacctccgcctttaccgggaaatcatcccggtttagattctacagatcttcccacaacagggggtagagatcctgagctactatatgctcagatgagacgtcgccctcgtggaagagatgagggaacgatttacgatgttccaagttctcaaaatagaaggcccggaacaggtgatgctagggattctatttacgacacgccaagacctgtctctgatggtatttacgacgtccccagatctccttccgaagatatttataatgtgccaagatctggccctcaactatttactgtgcttcctgaggatgggtataggcttccaaatctatcaggatctgctcttggagtgactccaggatttggaaatggtgttggggcagcttctatggcagaagaaattgataggtttattgaagaaacccatgaaagaagagagtcggcagcggcagcgcgtcgtcctttaccccctcttcctccgttgcaaactcctccggaaagtccttatggaagtaatcggatgatgcggttgttgagactcatgaacgatagggtacaggagtacaaagagcgtcgtaaggataagcaataa

SEQ ID NO: 26 - protein, 781 aa, Chlamydia psitacci
MAGVSGIGGGGGPGKLPPHGNDDDKQSKSASFGGHDIVFGDGERSRSGSVSSEHSIEERTRTLMEEGFQVRTPEEVEETRRASISPEEASNPGFFSRIWSSVKGIFTGGKKSDRAQGPEISSPIIAGYKRHGVRLPDARAMQAHLQSQSLQEISASDVSEIGDLDSGDTDITDISDESSLQSIDLDTDDRAEASTSSGRGVGGLAARVRGLWDFATRQQETPVDGFTGMTFSELVDTVQLYDQMILDADNETERQELLKYRDMYQSYVNTMLGEGNTSPTDQFDVSASAGIPGASSRRYSDGVGEARFLDIDDDLSSVSESELLDAIESGEYADHVLEEISPEVRRVLDEANNLRLQFDMEVSASVTPSLRERIQFALVRLERGIIRILTLIRRNLVALARLVRRGLRSLGELVRRCCVRERGVYRFLGRDRAYAREAERFIQRHTNSENFYSPGTLTVPYEVVNAWVNGRPDVVYVSDVRGMFGHEVVRLHVDDREGTYEIIGSSWIPYESDGGDTPPPLPGNHPSLDYADINDDSEDLPTTGDRDAEPLYAQMRPRPRGRDEGPIYDVPSPQSRRPRAGDDRDTPPPLPGNHPGLDSTDLPTTGGRDPELLYAQMRRRPRGRDEGTIYDVPSSQNRRPGTGDARDSIYDTPRPVSDGIYDVPRSPSEDIYNVPRSGPQLFTVLPEDGYRLPNLSGSALGVTPGFGNGVGAASMAEEIDRFIEETHERRESAAAARRPLPPLPPLQTPPESPYGSNRMMRLLRLMNDRVQEYKERRKDKQ

SEQ ID NO: 27 - DNA, 3042 bp, Chlamydia trachomatis
atgcaaacgtctttccataagttctttctttcaatgattctagcttattcttgctgctctttaagtgggggggggtatgcagcagaaatcatgattcctcaaggaatttacgatggggagacgttaactgtatcatttccctatactgttataggagatccgagtgggactactgttttttctgcaggagagttaacgttaaaaaatcttgacaattctattgcagctttgcctttaagttgttttgggaacttattagggagttttactgttttagggagaggacactcgttgactttcgagaacatacggacttctacaaatggagctgcactaagtgacagcgctaatagcgggttatttactattgagggttttaaagaattatctttttccaattgcaactcattacttgccgtactgcctgctgcaacgactaataatggtagccagactccgacgacaacatctacaccgtctaatggtactatttattctaaaacagatcttttgttactcaataatgagaagttctcattctatagtaatttagtctctggagatgggggagctatagatgctaagagcttaacggttcaaggaattagcaagctttgtgtcttccaagaaaatactgctcaagctgatgggggagcttgtcaagtagtcaccagtttctctgctatggctaacgaggctcctattgcctttatagcgaatgttgcaggagtaagagggggagggattgctgctgttcaggatgggcagcagggagtgtcatcatctacttcaacagaagatccagtagtaagtttttccagaaatactgcggtagagtttgatgggaacgtagcccgagtaggaggagggatttactcctacgggaacgttgctttcctgaataatggaaaaaccttgtttctcaacaatgttgcttctcctgtttacattgctgctgagcaaccaacaaatggacaggcttctaatacgagtgataattacggagatggaggagctatcttctgtaagaatggtgcgcaagcagcaggatccaataactctggatcagtttcctttgatggagagggagtagttttctttagtagcaatgtagctgctgggaaagggggagctatttatgccaaaaagctctcggttgctaactgtggccctgtacaattcttagggaatatcgctaatgatggtggagcgatttatttaggagaatctggagagctcagtttatctgctgattatggagatattattttcgatgggaatcttaaaagaacagccaaagagaatgctgccgatgttaatggcgtaactgtgtcctcacaagccatttcgatgggatcgggagggaaaataacgacattaagagctaaagcagggcatcagattctctttaatgatcccatcgagatggcaaacggaaataaccagccagcgcagtcttccgaacctctaaaaattaacgatggtgaaggatacacaggggatattgtttttgctaatggaaacagtactttgtaccaaaatgttacgatagagcaaggaaggattgttcttcgtgaaaaggcaaaattatcagtgaattctctaagtcagacaggtgggagtctgtatatggaagctgggagtacattggattttgtaactccacaaccaccacaacagcctcctgccgctaatcagttgatcacgctttccaatctgcatttgtctctttcttctttgttagcaaacaatgcagttacgaatcctcctaccaatcctccagcgcaagattctcatcctgcaatcattggtagcacaactgctggttctgttacaattagtgggcctatcttttttgaggatttggatgatacagcttatgataggtatgattggctaggttctaatcaaaaaatcgatgtcctgaaattacagttagggactcagccctcagctaatgccccatcagatttgactctagggaatgagatgcctaagtatggctatcaaggaagctggaagcttgcgtgggatcctaatacagcaaataatggtccttatactctgaaagctacatggactaaaactgggtataatcctgggcctgagcgagtagcttctttggttccaaatagtttatggggatccattttagatatacgatctgcgcattcagcaattcaagcaagtgtggatgggcgctcttattgtcgaggattatgggtttctggagtttcgaatttcttctatcatgaccgcgatgctttaggtcagggatatcggtatattagtgggggttattccttaggagcaaactcctactttggatcatcgatgtttggtctagcatttaccgaagtatttggtagatctaaagattatgtagtgtgtcgttccaatcatcatgcttgcataggatccgtttatctatctaccaaacaagctttatgtggatcctatttgttcggagatgcgtttatccgtgctagctacgggtttgggaaccagcatatgaaaacctcatacacatttgcagaggagagcgatgttcgttgggataataactgtctggttggagagattggagtgggattaccgattgtgattactccatctaagctctatttgaatgagttgcgtcctttcgtgcaagctgagttttcttatgccgatcatgaatcttttacagaggaaggcgatcaagctcgggcattcaggagtggacatctcatgaatctatcagttcctgttggagtaaaatttgatcgatgttctagtacacaccctaataaatatagctttatgggggcttatatctgtgatgcttatcgcaccatctctgggactcagacaacactcctatcccatcaagagacatggacaacagatgcctttcatttggcaagacatggagtcatagttagagggtctatgtatgcttctctaacaagcaatatagaagtatatggccatggaagatatgagtatcgagatacttctcgaggttatggtttgagtgcaggaagtaaagtccggttctaa

SEQ ID NO: 28 - protein, 1013 aa, Chlamydia trachomatis
MQTSFHKFFLSMILAYSCCSLSGGGYAAEIMIPQGIYDGETLTVSFPYTVIGDPSGTTVFSAGELTLKNLDNSIAALPLSCFGNLLGSFTVLGRGHSLTFENIRTSTNGAALSDSANSGLFTIEGFKELSFSNCNSLLAVLPAATTNNGSQTPTTTSTPSNGTIYSKTDLLLLNNEKFSFYSNLVSGDGGAIDAKSLTVQGISKLCVFQENTAQADGGACQVVTSFSAMANEAPIAFIANVAGVRGGGIAAVQDGQQGVSSSTSTEDPVVSFSRNTAVEFDGNVARVGGGIYSYGNVAFLNNGKTLFLNNVASPVYIAAEQPTNGQASNTSDNYGDGGAIFCKNGAQAAGSNNSGSVSFDGEGVVFFSSNVAAGKGGAIYAKKLSVANCGPVQFLGNIANDGGAIYLGESGELSLSADYGDIIFDGNLKRTAKENAADVNGVTVSSQAISMGSGGKITTLRAKAGHQILFNDPIEMANGNNQPAQSSEPLKINDGEGYTGDIVFANGNSTLYQNVTIEQGRIVLREKAKLSVNSLSQTGGSLYMEAGSTLDFVTPQPPQQPPAANQLITLSNLHLSLSSLLANNAVTNPPTNPPAQDSHPAIIGSTTAGSVTISGPIFFEDLDDTAYDRYDWLGSNQKIDVLKLQLGTQPSANAPSDLTLGNEMPKYGYQGSWKLAWDPNTANNGPYTLKATWTKTGYNPGPERVASLVPNSLWGSILDIRSAHSAIQASVDGRSYCRGLWVSGVSNFFYHDRDALGQGYRYISGGYSLGANSYFGSSMFGLAFTEVFGRSKDYVVCRSNHHACIGSVYLSTKQALCGSYLFGDAFIRASYGFGNQHMKTSYTFAEESDVRWDNNCLVGEIGVGLPIVITPSKLYLNELRPFVQAEFSYADHESFTEEGDQARAFRSGHLMNLSVPVGVKFDRCSSTHPNKYSFMGAYICDAYRTISGTQTTLLSHQETWTTDAFHLARHGVIVRGSMYASLTSNIEVYGHGRYEYRDTSRGYGLSAGSKVRF

SEQ ID NO: 29 - DNA, 2964 bp, Chlamydia muridarum
gtgatgcaaacgccttttcataagttctttcttctagcaatgctatcttactctttattgcaaggagggcatgcggcagatatttccatgcctccgggaatttatgatgggacaacattgacggcgccatttccctacactgtgatcggagatcccagagggacaaaggttacttcatcgggatcgctagagttgaaaaacctggacaattccattgcgactttacctctaagttgttttggtaatttgttggggaatttcactattgcaggaagagggcattcgttagtatttgagaatatacgaacatctacaaatggggcggcattgagtaatcatgctccttctggactgtttgtaattgaagcttttgatgaactctctcttttgaattgtaattcattggtatctgtagttcctcaaacagggggtacgactacttctgttccttctaatgggacgatctattctagaacagatcttgttctaagagatatcaagaaggtttctttctatagtaacttagtttctggagatgggggagctatagatgcacaaagtttaatggttaacggaattgaaaaactttgtaccttccaagaaaatgtagcgcagtccgatgggggagcgtgtcaggtaacaaagaccttctctgctgtgggcaataaggttcctttgtcttttttaggcaatgttgctggtaataaggggggaggagttgctgctgtcaaagatggtcagggggcaggaggggcgactgatctatcggttaattttgccaataatactgctgtagaatttgagggaaatagtgctcgaataggtggagggatctactcggacggaaatatttcctttttagggaatgcaaagacagttttcctaagtaacgtagcttcgcctatttatgttgaccctgctgctgcaggaggacagccccctgcagataaagataactatggagatggaggagccatcttctgcaaaaatgatactaacataggtgaagtctctttcaaagacgagggtgttgttttctttagtaaaaatattgccgcaggaaaggggggcgctatttatgctaagaaactgacaatttctgactgtggtccggtccagtttcttggtaatgtcgcgaatgacgggggcgctatttatctagtagatcagggggaacttagtctatctgctgatcgcggagatattatttttgatggaaatttaaagagaatggctacgcaaggcgctgccaccgtccatgatgtaatggttgcatcgaatgctatctctatggctacaggggggcaaatcacaacattaagggctaaggaaggtcgccgaattctttttaatgaccctattgaaatggcgaatggacaacctgtaatacaaactcttacagtaaacgagggcgaaggatatacgggggacattgtttttgctaaaggtgataatgttttgtactcaagtattgagctgagtcagggaagaattattctccgagagcaaacaaaattattggttaactccctgactcagactggagggagtgtacatatggaaggggggagtacactagactttgcagtaacaacgccaccagctgctaattcgatggctcttactaatgtacacttctccttagcttctttactaaaaaataatggggttacaaatcctccaacgaatcctccagtacaggtttctagtccagctgtaattggtaatacagctgctggtactgttacgatttctggtccgatcttttttgaagatttagatgaaactgcttacgataataatcagtggttaggtgcggatcaaactattgatgtgctgcagttgcatttaggagcgaatcctccggctaacgctccaactgatttgactttagggaacgaaagttctaaatatgggtatcaaggaagttggacacttcaatgggaaccagatcctgcgaatcctccacagaacaatagctacatgttgaaggcaagctggactaaaacaggttataatcctggtccggagcgcgtagcttctctggtctctaatagtctttggggatccattttagatgtgcgttccgcgcattctgcgattcaagcaagtatagatggacgagcttattgtcggggtatttggatttctgggatttcgaactttttctatcatgatcaggatgctttaggacaggggtatcgtcatattagtgggggatattcgataggagcaaactcttatttcgggtcttctatgtttggacttgcttttactgaaacttttggtaggtccaaagattatgtggtctgtcgatctaacgatcacacttgtgtaggctctgtttacttatccactagacaagcgttatgcggatcctgtttatttggagatgcttttgttcgggcgagttacggatttggaaatcagcatatgaagacctcttatacatttgctgaagagagtaatgtgcgttgggataataactgtgtagtgggagaagttggagctgggctccctatcatgctcgctgcatctaagctttatctaaatgagttgcgtcccttcgtgcaagcagagtttgcttatgcagagcatgaatcttttacagagagaggggatcaggctagggagtttaagagtgggcatcttatgaatctatctattccagttggggtgaagtttgatcgatgctctagtaaacatcctaacaagtatagttttatgggagcttatatctgtgatgcttaccggtccatttctggaacggagacaacactcctgtctcataaagagacttggacaacagatgctttccatttagcaaggcatggagttatggtcagaggatctatgtatgcttctttaacaggtaatatagaagtctatggccatggaaaatatgaatacagggatgcctctcgagggtatggtttaagtattggaagtaaaatccgattctaa

SEQ ID NO: 30 - protein, 987 aa, Chlamydia muridarum
MMQTPFHKFFLLAMLSYSLLQGGHAADISMPPGIYDGTTLTAPFPYTVIGDPRGTKVTSSGSLELKNLDNSIATLPLSCFGNLLGNFTIAGRGHSLVFENIRTSTNGAALSNHAPSGLFVIEAFDELSLLNCNSLVSVVPQTGGTTTSVPSNGTIYSRTDLVLRDIKKVSFYSNLVSGDGGAIDAQSLMVNGIEKLCTFQENVAQSDGGACQVTKTFSAVGNKVPLSFLGNVAGNKGGGVAAVKDGQGAGGATDLSVNFANNTAVEFEGNSARIGGGIYSDGNISFLGNAKTVFLSNVASPIYVDPAAAGGQPPADKDNYGDGGAIFCKNDTNIGEVSFKDEGVVFFSKNIAAGKGGAIYAKKLTISDCGPVQFLGNVANDGGAIYLVDQGELSLSADRGDIIFDGNLKRMATQGAATVHDVMVASNAISMATGGQITTLRAKEGRRILFNDPIEMANGQPVIQTLTVNEGEGYTGDIVFAKGDNVLYSSIELSQGRIILREQTKLLVNSLTQTGGSVHMEGGSTLDFAVTTPPAANSMALTNVHFSLASLLKNNGVTNPPTNPPVQVSSPAVIGNTAAGTVTISGPIFFEDLDETAYDNNQWLGADQTIDVLQLHLGANPPANAPTDLTLGNESSKYGYQGSWTLQWEPDPANPPQNNSYMLKASWTKTGYNPGPERVASLVSNSLWGSILDVRSAHSAIQASIDGRAYCRGIWISGISNFFYHDQDALGQGYRHISGGYSIGANSYFGSSMFGLAFTETFGRSKDYVVCRSNDHTCVGSVYLSTRQALCGSCLFGDAFVRASYGFGNQHMKTSYTFAEESNVRWDNNCVVGEVGAGLPIMLAASKLYLNELRPFVQAEFAYAEHESFTERGDQAREFKSGHLMNLSIPVGVKFDRCSSKHPNKYSFMGAYICDAYRSISGTETTLLSHKETWTTDAFHLARHGVMVRGSMYASLTGNIEVYGHGKYEYRDASRGYGLSIGSKIRF

SEQ ID NO: 31 - DNA, 3036 bp, Chlamydia psitacci
atgaaagcgtctcttcgtaagtttctaatttcaacaacgctaacacttccatactcattccaagccttttctttggaagtcgtagtccctaatggaacttatgatgggaaccttagagaaacgttcccctatacgattacatccaatcctgaaggaacaacggcaatactgtcaggaaatttaaatcttttaaatcttgataactccatggtagcaacgccttcaagttgctttttcaactctgcaggatccatgacaattgtggggagaaaccacaatctaacctttacaaaccttcgcacgtcggcaaacggtgctgccctaagctctattcctacaacaactcctgaatcgttcccttatacgattaaaggagtgaacaccctctccttttctaactgcctagccctaatggcccgcacaacaacggcgccaaatacgacaactcctgtaaatccaaacggaggggcgttctactccaaagctcctgtatttctagagaatattcagaatgtgctatttaaaaataacagggctgctgatagcggcggtggcctatgggtagaaacagctgggattagcaatatcaaaaaatccatgcagttccttagcaacgtcggagccaacggtggcgctatcaacgcgtctaaaagcctagatgttacgcaatgtccttcgattctcttcagatctaactctgctgagaaactcggaggagctatccaagctgttgatcctgcaacaacaaatcaagtaaatactgccgtcagattctccgaaaatggctcagtacaatttgatgccaataatgcgaaatctggcggagcgatttattcgaaagggaacgtcgatttctcaaataatgcgcaattgctgatacagaataactccgcatctcctgaagtcgctaatactaatgaagtattaggacaaggtggggcgattttctgtgtacaacagactcctactcaaccgccgccgccaccaccacctacaacgaatcctgtcttctcaggattaactataaccaatcaaaaagatatcctctttgcaaataactttgccgcaactgcaggcggagcgatttatggagaaaaggtcagcattacctcctcagggaaaacgatgtttacaaacaacatcgctaaagatggcggagctatctacattcctgaaaatggagagctcaccttatctgctgactacggcgatatgattttctatgaaaatctaaaaaaagatgacgctactgtcacaagaaacgctgttaccttagcaaaaggagcaacgattaagttactagcagcttctggggatcataaactctgtttctacgatcctattgtgactacacttccagaaacagctcctactaatgacaagactctaacgatcaaccaagataaaacatcctccaccccttttactaactatattggaaccctcctattttcaggagcttatgtagatagccaaagcgctagcactacagcgaattttgaatccactatctatcaaaaagtcatcttaggtggcgggaagcttgttctagcagataaagccagcctatctgtagcttcctttactcaggaaacagattctattcttttaatggataatggaactactctagcaattacagagcattcccatcaaacaccagcagctggtgggggtggcggaggcggaggaacccccactcaggaagccaatactgatggagttatttccttaacaaatcttcatgtcaatatcagctcgcttacggaacaaggtgagggggcgaaacttgaaacaaaaaatacagatgggacgataactttaactgggcatgtatccttagacgatgtttcaggaactgcttacgagaatcacgatcttttcaataaagataccgtcacgataaatctgctttctctttctacagcaggagatagtaaaacgacgatcaatggtttggacctcactcttagaggagacgcagaacctcaatacggttaccaaggatcatggcaactggcttgggaaaatggagctgatgccaataaacagaaaatcctaaaagctacatggacaaaaacaggattcactcctaatcctgagcgtcaagcatctttagttcctaatagcttatggggagcattcatcgacctacgttctatgaatgccttagcgacagcaagctgtgacggcttcggttatggtaagggattgtgggtagctgggatttccaatatcttccaccatgatcgcaatagcgtatcccatggtttccgtcgtattagcggtggttatgttattggagccaattcacaaacagtaacggattctgtatttggagtggccttctcccagatatttgctaagtctaaagactatgttgtctcctcagcaaaatcacaagctatagcaggtagcgcttacctatcggtaaaacgtcagttaagcaacacgatattctcatccttcgctgcaagaattaactacagccatactaacgaggatatgaaaacacgctataccttcattcctgaaaaagatggcaattgggataataactgctggttaggagaaataggcggaagcttacctattgttttacaaattactaaattacatctaaatcaaatcattccttttatgaatgttcagcttggctatgctgagcatggatcgtttaaagaaaaacttgcagaagcacgctccttctgttcttctcgtttgattaacttagcggttcctgttggatttaaaattgataggcgttcccactcccatccggatttttacagcctagctatatcctacattcccgatgtatggcgaaggaatccaggatgtaacactttattgctcgcaaatggagtccgttggaaaacgccagcaactaatctaaatagacatggtttattgatgcaaggatccacacatacagctgtgctcagtaatattgagatctttagccatggtagttgcgaattacgtagctcctcacgcaactacaatataaatgtaggaagtaaaattcgattctaa

SEQ ID NO: 32 - protein, 1011 aa, Chlamydia psitacci
MKASLRKFLISTTLTLPYSFQAFSLEVVVPNGTYDGNLRETFPYTITSNPEGTTAILSGNLNLLNLDNSMVATPSSCFFNSAGSMTIVGRNHNLTFTNLRTSANGAALSSIPTTTPESFPYTIKGVNTLSFSNCLALMARTTTAPNTTTPVNPNGGAFYSKAPVFLENIQNVLFKNNRAADSGGGLWVETAGISNIKKSMQFLSNVGANGGAINASKSLDVTQCPSILFRSNSAEKLGGAIQAVDPATTNQVNTAVRFSENGSVQFDANNAKSGGAIYSKGNVDFSNNAQLLIQNNSASPEVANTNEVLGQGGAIFCVQQTPTQPPPPPPPTTNPVFSGLTITNQKDILFANNFAATAGGAIYGEKVSITSSGKTMFTNNIAKDGGAIYIPENGELTLSADYGDMIFYENLKKDDATVTRNAVTLAKGATIKLLAASGDHKLCFYDPIVTTLPETAPTNDKTLTINQDKTSSTPFTNYIGTLLFSGAYVDSQSASTTANFESTIYQKVILGGGKLVLADKASLSVASFTQETDSILLMDNGTTLAITEHSHQTPAAGGGGGGGGTPTQEANTDGVISLTNLHVNISSLTEQGEGAKLETKNTDGTITLTGHVSLDDVSGTAYENHDLFNKDTVTINLLSLSTAGDSKTTINGLDLTLRGDAEPQYGYQGSWQLAWENGADANKQKILKATWTKTGFTPNPERQASLVPNSLWGAFIDLRSMNALATASCDGFGYGKGLWVAGISNIFHHDRNSVSHGFRRISGGYVIGANSQTVTDSVFGVAFSQIFAKSKDYVVSSAKSQAIAGSAYLSVKRQLSNTIFSSFAARINYSHTNEDMKTRYTFIPEKDGNWDNNCWLGEIGGSLPIVLQITKLHLNQIIPFMNVQLGYAEHGSFKEKLAEARSFCSSRLINLAVPVGFKIDRRSHSHPDFYSLAISYIPDVWRRNPGCNTLLLANGVRWKTPATNLNRHGLLMQGSTHTAVLSNIEIFSHGSCELRSSSRNYNINVGSKIRF

SEQ ID NO: 33 - DNA, 1806 bp, Chlamydia trachomatis
atgaaaatgaataggatttggctattactgcttaccttttcttctgccatacattctcctgtacaaggagaaagcttggtttgcaagaatgctcttcaagatttgagttttttagagcatttattacaggttaaatatgctcctaaaacatggaaagagcaatacttaggatgggatcttgttcaaagctccgtttctgcacagcagaagcttcgtacacaagaaaatccatcaacaagtttttgccagcaggtccttgctgattttatcggaggattaaatgactttcacgctggagtaactttctttgcgatagaaagtgcttaccttccttataccgtacaaaaaagtagtgacggccgtttctactttgtagatatcatgactttttcttcagagatccgtgttggagatgagttgctagaggtggatggggcgcctgtccaagatgtactcgctactctatatggaagcaatcacaaagggactgcagctgaagagtcggctgctttaagaacactattttctcgcatggcctctttagggcacaaagtaccttctgggcgcactactttaaagattcgtcgtccttttggtactacgagagaagttcgtgtgaaatggcgttatgttcctgaaggtgtaggagatttggctaccatagctccttctatcagggctccacagttacagaaatcgatgagaagctttttccctaagaaagatgatgcgtttcatcggtctagttcgctattctactctccaatggttccgcatttttgggcagagcttcgcaatcattatgcaacgagtggtttgaaaagcgggtacaatattgggagtaccgatgggtttctccctgtcattgggcctgttatatgggagtcggagggtcttttccgcgcttatatttcttcggtgactgatggggatggtaagagccataaagtaggatttctaagaattcctacatatagttggcaggacatggaagattttgatccttcaggaccgcctccttgggaagaatttgctaagattattcaagtattttcttctaatacagaagctttgattatcgaccaaacgaacaacccaggtggtagtgtcctttatctttatgcactgctttccatgttgacagaccgtcctttagaacttcctaaacatagaatgattctgactcaggatgaagtggttgatgctttagattggttaaccctgttggaaaacgtagacacaaacgtggagtctcgccttgctctgggagacaacatggaaggatatactgtggatctacaggttgccgagtatttaaaaagctttggacgtcaagtattgaattgttggagtaaaggggatatcgagttatcaacgcctattcctctttttggttttgagaagattcatccacatcctcgagttcaatactctaaaccgatttgtgttttgatcaatgagcaagacttttcttgtgctgacttcttccctgtagttttgaaagacaatgatcgagctcttattgttggtactcgaacagctggagctggaggatttgtctttaatgtgcagttcccaaatagaactggaataaaaacttgttctttaacaggatcattagctgttagagagcatggtgccttcattgagaacatcggagtcgaaccgcatatcgatctgccttttacagcgaatgatattcgctataaaggctattccgagtatcttgataaggtcaaaaaattggtttgtcagctgatcaataacgacggtaccattattcttgcggaagatggtagtttttaa

SEQ ID NO: 34 - protein, 601 aa, Chlamydia trachomatis
MKMNRIWLLLLTFSSAIHSPVQGESLVCKNALQDLSFLEHLLQVKYAPKTWKEQYLGWDLVQSSVSAQQKLRTQENPSTSFCQQVLADFIGGLNDFHAGVTFFAIESAYLPYTVQKSSDGRFYFVDIMTFSSEIRVGDELLEVDGAPVQDVLATLYGSNHKGTAAEESAALRTLFSRMASLGHKVPSGRTTLKIRRPFGTTREVRVKWRYVPEGVGDLATIAPSIRAPQLQKSMRSFFPKKDDAFHRSSSLFYSPMVPHFWAELRNHYATSGLKSGYNIGSTDGFLPVIGPVIWESEGLFRAYISSVTDGDGKSHKVGFLRIPTYSWQDMEDFDPSGPPPWEEFAKIIQVFSSNTEALIIDQTNNPGGSVLYLYALLSMLTDRPLELPKHRMILTQDEVVDALDWLTLLENVDTNVESRLALGDNMEGYTVDLQVAEYLKSFGRQVLNCWSKGDIELSTPIPLFGFEKIHPHPRVQYSKPICVLINEQDFSCADFFPVVLKDNDRALIVGTRTAGAGGFVFNVQFPNRTGIKTCSLTGSLAVREHGAFIENIGVEPHIDLPFTANDIRYKGYSEYLDKVKKLVCQLINNDGTIILAEDGSF

SEQ ID NO: 35 - DNA, 1806 bp, Chlamydia muridarum
atgaaaatgaataggattttgctactgctgctaaccttttcttccgctatacattctcctttgcatggggaaagtttagtctgtcagaatgctctgaaagatttgagttttttggagcatttgctgcaagtcaagtatgcccctaaaacttggaaagaacagtatttaggttgggatctttctaaaagctctgtttttgcagagcagaaattgcgttccgaggacaacccttcaacaagcttttgtcagcaagtaattgcggactttattggagcgttgagtgattttcatgccggggtctctttctttgctgtagagagtgcctaccttccctactctgtacaaaaaagtagcgatggacgcttctatttcgttgatgtaatgaccttttcttctgatattcgcgtcggggatgagttacttgaggtagacgggcagcctgttgcagaagcacttgctaccctatatggaaccaatcacaaggggactctcgctgaagaatctgctgctttaagaacgttattttctcgtatggcttctttaggacataaagtcccttccgggagaatcaccctcaaagttcgtcgttcttctggttctgtgaaagatgtgcgagcaaaatggcgttatactccagaaagtgtaggggatttagctacgatagctccttccataaaagctccacagctgcagaagtctatgagaggggcctttcctaaaaaagaaagtgtatttcatcagtcgagcactctgttttattctccaatggttcctcatttttggtcggagtttcgtaatcactacgcaacgagtggtttaaaaagtgggtacaatattggggataccgatggatttttcccagtcatgggacccgttatttgggagtcggacggaatttttcatgcttatattttccccttggttgatgaaaatggtagaagccataacgtaggatttatcagaattcctacgtatggttggcaagagatggaagatttagattctatagggacacctccttgggaagagtttggtaagatcattacgctattttctgaaaaaacagaggctttgatcattgaccaaacgaataatcctggggggagcgttatgtatttatacggattgctctctatgttgacggataaacctttagatcttcctaaacatagaatgattctaactcaggacgaagtagttgatgctttagattggttgaatttattggaaaatgtggatacaaacgcagaggctcggattgctttgggagataatatggaaggatatcccattgacttgcaggctgctgaatatctgaaaagctttgctcatcaggtattggcatgttggaagaatggagatatcgaattatctacaccgattcctctttttgggtttgagaaaattcatccacatcctcgagtccaatatactaagcctatttgtgttttgattaatgaacaggatttttcttgtgcggatttcttccctgctattctgaaagacaatgacagagcccttgtcgttggaactcgaacagcgggagctgggggatttgtcttcaatgtacaattccctaacagaacgggaattaaaagttgctctttaacaggatctttagcagttagagagcatggggatttgattgaaaatgttggggttgaacctcatattgaaattcctttcacagctaatgatattcgttatagagggtattctgaatatattcagaaagtacaaaaattggttgctcagctaatcaataatgacagtgtaattattctctcagaggatggaagtttttaa

SEQ ID NO: 36 - protein, 601 aa, Chlamydia muridarum
MKMNRILLLLLTFSSAIHSPLHGESLVCQNALKDLSFLEHLLQVKYAPKTWKEQYLGWDLSKSSVFAEQKLRSEDNPSTSFCQQVIADFIGALSDFHAGVSFFAVESAYLPYSVQKSSDGRFYFVDVMTFSSDIRVGDELLEVDGQPVAEALATLYGTNHKGTLAEESAALRTLFSRMASLGHKVPSGRITLKVRRSSGSVKDVRAKWRYTPESVGDLATIAPSIKAPQLQKSMRGAFPKKESVFHQSSTLFYSPMVPHFWSEFRNHYATSGLKSGYNIGDTDGFFPVMGPVIWESDGIFHAYIFPLVDENGRSHNVGFIRIPTYGWQEMEDLDSIGTPPWEEFGKIITLFSEKTEALIIDQTNNPGGSVMYLYGLLSMLTDKPLDLPKHRMILTQDEVVDALDWLNLLENVDTNAEARIALGDNMEGYPIDLQAAEYLKSFAHQVLACWKNGDIELSTPIPLFGFEKIHPHPRVQYTKPICVLINEQDFSCADFFPAILKDNDRALVVGTRTAGAGGFVFNVQFPNRTGIKSCSLTGSLAVREHGDLIENVGVEPHIEIPFTANDIRYRGYSEYIQKVQKLVAQLINNDSVIILSEDGSF

SEQ ID NO: 37 - DNA, 1782 bp, Chlamydia psitacci
atgaaagtgaaacaaattacagccttgatttgctccttagtattaggttttcaaatttcaggttccgctaagactttagttcaaaaaaatgcatgttctgacttggattttttggaacacttacttgatgttaaatatgctcctaaagagtggaaacataagctttttcattgggatttgaaagatgcaacggatcaagcacgtttaaaattgtgtattgaggaaaatccttcaacaagctactgccaaggagttcttgcagaatatatctctgatttaaaagattttcatgccgggattactttcttccgcacagagaattctcatctaccttatacggttaagttaagcaattctcgcagatgctttattgttgatgtgcatacgtataactctgaaatttctgtaggtgatgaaattttagagatggacggtatgccgattatggaggtaatcgagagtatacgtactggtagaggagctctttctgattacgctgcagctgcacgaacactcttttctcgttctgctgccttaggccatcaaattcctatgggagtggcaacattaaaaattcgtcgtcctagcggtttaacgcgtacggtaaaagctaaatggcgccatacgcctgaatatattcaagatctatctttaatatctcctttagtaaaagatcctatcatccagatgagatctagccgtgcttgccctttattatctagtgcttctgaaaattgtttattcacaaacgaaatggttccttatttctggaaggaattacgtcagcaatataaacgtggtttaagtagtgattacaatatcggaagtaaaagaggtttcttacctgattttggacatgtgacatggaaagctaaaagtggtccttaccatgcttatgtgttcacctgcaccgataatcatggacagtctcacagtattggattccttaggatttctacatattcttggacagatatggaagatcgtactgctatgaatatggaatccccatgggatgacttcagcgagatcattagtgttttacaagagaaaaccgaagctttgattatcgatcagacaaacaatcctggtggtagtgtcttctatctttatgcattgatttctagattaacagacagacctttagaaacacctaaacatagaatgattctcactcaaagtgaagtacaatctgcagtacaatggttgaaccttcttgaaggagttgaaaccgatgagcaagcaagaaatgctctcggtgaggatatggaaggttatcctatcgatatgaatgcggcaggatatctacagacattttctaatactgttttaaaatgttgggcaaatggggatattaatctctctacacctatgcctttgttaggatttgctaaagtacatccacatcctgaacatcgttatacacgtcctatctgcgtcttaatcaatcaggaagatttctcctgcggagatttattccctgcgattatgaaggatagcggtcgagctcttattgtaggaacggccacagcaggagccggaggttttgtctttaacgtagagttccctaatagaacaggcattaaaagttgttctttaacaggatctctagcagtaagacctgacggttcttacatagagaatttaggggtctctcctcatatattcttagattttacagatacggatgtacaaacaggaaaatattctgattacattagcactgtgaaaagtttagttcttgatcttattgaaagagaagctgataacaaagcttcttaa

SEQ ID NO: 38 - protein, 593 aa, Chlamydia psitacci
MKVKQITALICSLVLGFQISGSAKTLVQKNACSDLDFLEHLLDVKYAPKEWKHKLFHWDLKDATDQARLKLCIEENPSTSYCQGVLAEYISDLKDFHAGITFFRTENSHLPYTVKLSNSRRCFIVDVHTYNSEISVGDEILEMDGMPIMEVIESIRTGRGALSDYAAAARTLFSRSAALGHQIPMGVATLKIRRPSGLTRTVKAKWRHTPEYIQDLSLISPLVKDPIIQMRSSRACPLLSSASENCLFTNEMVPYFWKELRQQYKRGLSSDYNIGSKRGFLPDFGHVTWKAKSGPYHAYVFTCTDNHGQSHSIGFLRISTYSWTDMEDRTAMNMESPWDDFSEIISVLQEKTEALIIDQTNNPGGSVFYLYALISRLTDRPLETPKHRMILTQSEVQSAVQWLNLLEGVETDEQARNALGEDMEGYPIDMNAAGYLQTFSNTVLKCWANGDINLSTPMPLLGFAKVHPHPEHRYTRPICVLINQEDFSCGDLFPAIMKDSGRALIVGTATAGAGGFVFNVEFPNRTGIKSCSLTGSLAVRPDGSYIENLGVSPHIFLDFTDTDVQTGKYSDYISTVKSLVLDLIEREADNKAS

SEQ ID NO: 39 - DNA, 1860 bp, Chlamydia pneumoniae
atgaaaaaagggaaattaggagccatagtttttggccttctatttacaagtagtgttgctggtttttctaaggatttgactaaagacaacgcttatcaagatttaaatgtcatagagcatttaatatcgttaaaatatgctcctttaccatggaaggaactattatttggttgggatttatctcagcaaacacagcaagctcgcttgcaactggtcttagaagaaaaaccaacaaccaactactgccagaaggtactctctaactacgtgagatcattaaacgattatcatgcagggattacgttttatcgtactgaaagtgcgtatatcccttacgtattgaagttaagtgaagatggtcatgtctttgtagtcgacgtacagactagccaaggggatatttacttaggggatgaaatccttgaagtagatggaatggggattcgtgaggctatcgaaagccttcgctttggacgagggagtgccacagactattctgctgcagttcgttccttgacatcgcgttccgccgcttttggagatgcggttccttcaggaattgccatgttgaaacttcgccgacccagtggtttgatccgttcgacaccggtccgttggcgttatactccagagcatatcggagatttttctttagttgctcctttgattcctgaacataaacctcaattacctacacaaagttgtgtgctattccgttccggggtaaattcacagtcttctagtagctctttattcagttcctacatggtgccttatttctgggaagaattgcgggttcaaaataagcagcgttttgacagtaatcaccatatagggagccgtaatggatttttacctacgtttggtcctattctttgggaacaagacaaggggccctatcgttcctatatctttaaagcaaaagattctcagggcaatccccatcgcataggatttttaagaatttcttcttatgtttggactgatttagaaggacttgaagaggatcataaggatagtccttgggagctctttggagagatcatcgatcatttggaaaaagagactgatgctttgattattgatcagacccataatcctggaggcagtgttttctatctctattcgttactatctatgttaacagatcatcctttagatactcctaaacatagaatgattttcactcaggatgaagtcagctcggctttgcactggcaagatctactagaagatgtcttcacagatgagcaggcagttgccgtgctaggggaaactatggaaggatattgcatggatatgcatgctgtagcctctcttcaaaacttctctcagagtgtcctttcttcctgggtttcaggtgatattaacctttcaaaacctatgcctttgctaggatttgcacaggttcgacctcatcctaaacatcaatatactaaacctttgtttatgttgatagacgaggatgacttctcttgtggagatttagcgcctgcaattttgaaggataatggccgcgctactctcattggaaagccaacagcaggagctggaggttttgtattccaagtcactttccctaaccgttctggaattaaaggtctttctttaacaggatctttagctgttaggaaagatggtgagtttattgaaaacttaggagtggctcctcatattgatttaggatttacctccagggatttgcaaacttccaggtttactgattacgttgaggcagtgaaaactatagttttaacttctttgtctgagaacgctaagaagagtgaagagcagacttctccgcaagagacgcctgaagttattcgagtctcttatcccacaacgacttctgcttcgtaa

SEQ ID NO: 40 - protein, 619 aa, Chlamydia pneumoniae
MKKGKLGAIVFGLLFTSSVAGFSKDLTKDNAYQDLNVIEHLISLKYAPLPWKELLFGWDLSQQTQQARLQLVLEEKPTTNYCQKVLSNYVRSLNDYHAGITFYRTESAYIPYVLKLSEDGHVFVVDVQTSQGDIYLGDEILEVDGMGIREAIESLRFGRGSATDYSAAVRSLTSRSAAFGDAVPSGIAMLKLRRPSGLIRSTPVRWRYTPEHIGDFSLVAPLIPEHKPQLPTQSCVLFRSGVNSQSSSSSLFSSYMVPYFWEELRVQNKQRFDSNHHIGSRNGFLPTFGPILWEQDKGPYRSYIFKAKDSQGNPHRIGFLRISSYVWTDLEGLEEDHKDSPWELFGEIIDHLEKETDALIIDQTHNPGGSVFYLYSLLSMLTDHPLDTPKHRMIFTQDEVSSALHWQDLLEDVFTDEQAVAVLGETMEGYCMDMHAVASLQNFSQSVLSSWVSGDINLSKPMPLLGFAQVRPHPKHQYTKPLFMLIDEDDFSCGDLAPAILKDNGRATLIGKPTAGAGGFVFQVTFPNRSGIKGLSLTGSLAVRKDGEFIENLGVAPHIDLGFTSRDLQTSRFTDYVEAVKTIVLTSLSENAKKSEEQTSPQETPEVIRVSYPTTTSAS

SEQ ID NO: 41 - DNA, 4596 bp, Chlamydia trachomatis
atgagttccgagaaagatataaaaagcacctgttctaagttttctttgtctgtagtagcagctatccttgcctctgttagcgggttagctagttgcgtagatcttcatgctggaggacagtctgtaaatgagctggtatatgtaggccctcaagcggttttattgttagaccaaattcgagatctattcgttgggtctaaagatagtcaggctgaaggacagtataggttaattgtaggagatccaagttctttccaagagaaagatgcggatactcttcccgggaaggtagagcaaagtactttgttctcagtaaccaatcccgtggttttccaaggtgtggaccaacaggatcaagtctcttcccaagggttaatttgtagttttacgagcagcaaccttgattctcctcgtgacggagaatcttttttaggtattgcttttgttggggatagtagtaaggctggaatcacattaactgacgtgaaagcttctttgtctggagcggctttatattctacagaagatcttatctttgaaaagattaagggtggattggaatttgcatcatgttcttctctagaacaggggggagcttgtgcagctcaaagtattttgattcatgattgtcaaggattgcaggttaaacactgtactacagccgtgaatgctgaggggtctagtgcgaatgatcatcttggatttggaggaggcgctttctttgttacgggttctctttctggagagaaaagtctctatatgcctgcaggagatatggtagttgcgaattgtgatggggctatatcttttgaaggaaacagcgcgaactttgctaatggaggagcgattgctgcctctgggaaagtgctttttgtcgctaatgataaaaagacttcttttatagagaaccgagctttgtctggaggagcgattgcagcctcttctgatattgcctttcaaaactgcgcagaactagttttcaaaggcaattgtgcaattggaacagaggataaaggttctttaggtggaggggctatatcttctctaggcaccgttcttttgcaagggaatcacgggataacttgtgataagaatgagtctgcttcgcaaggaggcgccatttttggcaaaaattgtcagatttctgacaacgaggggccagtggttttcagagatagtacagcttgcttaggaggaggcgctattgcagctcaagaaattgtttctattcagaacaatcaggctgggatttccttcgagggaggtaaggctagtttcggaggaggtattgcgtgtggatctttttcttccgcaggtggtgcttctgttttagggaccattgatatttcgaagaatttaggcgcgatttcgttctctcgtactttatgtacgacctcagatttaggacaaatggagtaccagggaggaggagctctatttggtgaaaatatttctctttctgagaatgctggtgtgctcacctttaaagacaacattgtgaagacttttgcttcgaatgggaaaattctgggaggaggagcgattttagctactggtaaggtggaaattactaataattccgaaggaatttcttttacaggaaatgcgagagctccacaagctcttccaactcaagaggagtttcctttattcagcaaaaaagaagggcgaccactctcttcaggatattctgggggaggagcgattttaggaagagaagtagctattctccacaacgctgcagtagtatttgagcaaaatcgtttgcagtgcagcgaagaagaagcgacattattaggttgttgtggaggaggcgctgttcatgggatggatagcacttcgattgttggcaactcttcagtaagatttggtaataattacgcaatgggacaaggagtctcaggaggagctcttttatctaaaacagtgcagttagctgggaatggaagcgtcgatttttctcgaaatattgctagtttgggaggaggagctcttcaagcttctgaaggaaattgtgagctagttgataacggctatgtgctattcagagataatcgagggagggtttatgggggtgctatttcttgcttacgtggagatgtagtcatttctggaaacaagggtagagttgaatttaaagacaacatagcaacacgtctttatgtggaagaaactgtagaaaaggttgaagaggtagagccagctcctgagcaaaaagacaataatgagctttctttcttagggagagcagaacagagttttattactgcagctaatcaagctcttttcgcatctgaagatggggatttatcacctgagtcatccatttcttctgaagaacttgcgaaaagaagagagtgtgctggaggagctatttttgcaaaacgggttcgtattgtagataaccaagaggccgttgtattctcgaataacttctctgatatttatggcggcgccatttttacaggttctcttcgagaagaggataagttagatgggcaaatccctgaagtcttgatctcaggcaatgcaggggatgttgttttttccggaaattcctcgaagcgtgatgagcatcttcctcatacaggtgggggagccatttgtactcaaaatttgacgatttctcagaatacagggaatgttctgttttataacaacgtggcctgttcgggaggagctgttcgtatagaggatcatggtaatgttcttttagaagcttttggaggagatattgtttttaaaggaaattcttctttcagagcacaaggatccgatgctatctattttgcaggtaaagaatcgcatattacagccctgaatgctacggaaggacatgctattgttttccacgacgcattagtttttgaaaatctagaagaaaggaaatctgctgaagtattgttaatcaatagtcgagaaaatccaggttacactggatctattcgatttttagaagcagaaagtaaagttcctcaatgtattcatgtacaacaaggaagccttgagttgctaaatggagccacattatgtagttatggttttaaacaagatgctggagctaagttggtattggctgctggagctaaactgaagattttagattcaggaactcctgtacaacaagggcatgctatcagtaaacctgaagcagaaatcgagtcatcttctgaaccagagggtgcacattctctttggattgcgaagaatgctcaaacaacagttcctatggttgatatccatactatttctgtagatttagcctccttctcttctagtcaacaggaggggacagtagaagctcctcaggttattgttcctggaggaagttatgttcgatctggagagcttaatttggagttagttaacacaacaggtactggttatgaaaatcatgctttattgaagaatgaggctaaagttccattgatgtctttcgttgcttctggtgatgaagcttcagccgaaatcagtaacttgtcggtttctgatttacagattcatgtagtaactccagagattgaagaagacacatacggccatatgggagattggtctgaggctaaaattcaagatggaactcttgtcattagttggaatcctactggatatcgattagatcctcaaaaagcaggggctttagtatttaatgcattatgggaagaaggggctgtcttgtctgctctgaaaaatgcacgctttgctcataatctcactgctcagcgtatggaattcgattattctacaaatgtgtggggattcgcctttggtggtttccgaactctatctgcagagaatctggttgctattgatggatacaaaggagcttatggtggtgcttctgctggagtcgatattcaattgatggaagattttgttctaggagttagtggagctgctttcctaggtaaaatggatagtcagaagtttgatgcggaggtttctcggaagggagttgttggttctgtatatacaggatttttagctggatcctggttcttcaaaggacaatatagccttggagaaacacagaacgatatgaaaacgcgttatggagtactaggagagtcgagtgcttcttggacatctcgaggagtactggcagatgctttagttgaataccgaagtttagttggtcctgtgagacctactttttatgctttgcatttcaatccttatgtcgaagtatcttatgcttctatgaaattccctggctttacagaacaaggaagagaagcgcgttcttttgaagacgcttcccttaccaatatcaccattcctttagggatgaagtttgaattggcgttcataaaaggacagttttcagaggtgaactctttgggaataagttatgcatgggaagcttatcgaaaagtagaaggaggcgcggtgcagcttttagaagctgggtttgattgggagggagctccaatggatcttcctagacaggagctgcgtgtcgctctggaaaataatacggaatggagttcttacttcagcacagtcttaggattaacagctttttgtggaggatttacttctacagatagtaaactaggatatgaggcgaatactggattgcgattgatcttttaa

SEQ ID NO: 42 - protein, 1531 aa, Chlamydia trachomatis
MSSEKDIKSTCSKFSLSVVAAILASVSGLASCVDLHAGGQSVNELVYVGPQAVLLLDQIRDLFVGSKDSQAEGQYRLIVGDPSSFQEKDADTLPGKVEQSTLFSVTNPVVFQGVDQQDQVSSQGLICSFTSSNLDSPRDGESFLGIAFVGDSSKAGITLTDVKASLSGAALYSTEDLIFEKIKGGLEFASCSSLEQGGACAAQSILIHDCQGLQVKHCTTAVNAEGSSANDHLGFGGGAFFVTGSLSGEKSLYMPAGDMVVANCDGAISFEGNSANFANGGAIAASGKVLFVANDKKTSFIENRALSGGAIAASSDIAFQNCAELVFKGNCAIGTEDKGSLGGGAISSLGTVLLQGNHGITCDKNESASQGGAIFGKNCQISDNEGPVVFRDSTACLGGGAIAAQEIVSIQNNQAGISFEGGKASFGGGIACGSFSSAGGASVLGTIDISKNLGAISFSRTLCTTSDLGQMEYQGGGALFGENISLSENAGVLTFKDNIVKTFASNGKILGGGAILATGKVEITNNSEGISFTGNARAPQALPTQEEFPLFSKKEGRPLSSGYSGGGAILGREVAILHNAAVVFEQNRLQCSEEEATLLGCCGGGAVHGMDSTSIVGNSSVRFGNNYAMGQGVSGGALLSKTVQLAGNGSVDFSRNIASLGGGALQASEGNCELVDNGYVLFRDNRGRVYGGAISCLRGDVVISGNKGRVEFKDNIATRLYVEETVEKVEEVEPAPEQKDNNELSFLGRAEQSFITAANQALFASEDGDLSPESSISSEELAKRRECAGGAIFAKRVRIVDNQEAVVFSNNFSDIYGGAIFTGSLREEDKLDGQIPEVLISGNAGDVVFSGNSSKRDEHLPHTGGGAICTQNLTISQNTGNVLFYNNVACSGGAVRIEDHGNVLLEAFGGDIVFKGNSSFRAQGSDAIYFAGKESHITALNATEGHAIVFHDALVFENLEERKSAEVLLINSRENPGYTGSIRFLEAESKVPQCIHVQQGSLELLNGATLCSYGFKQDAGAKLVLAAGAKLKILDSGTPVQQGHAISKPEAEIESSSEPEGAHSLWIAKNAQTTVPMVDIHTISVDLASFSSSQQEGTVEAPQVIVPGGSYVRSGELNLELVNTTGTGYENHALLKNEAKVPLMSFVASGDEASAEISNLSVSDLQIHVVTPEIEEDTYGHMGDWSEAKIQDGTLVISWNPTGYRLDPQKAGALVFNALWEEGAVLSALKNARFAHNLTAQRMEFDYSTNVWGFAFGGFRTLSAENLVAIDGYKGAYGGASAGVDIQLMEDFVLGVSGAAFLGKMDSQKFDAEVSRKGVVGSVYTGFLAGSWFFKGQYSLGETQNDMKTRYGVLGESSASWTSRGVLADALVEYRSLVGPVRPTFYALHFNPYVEVSYASMKFPGFTEQGREARSFEDASLTNITIPLGMKFELAFIKGQFSEVNSLGISYAWEAYRKVEGGAVQLLEAGFDWEGAPMDLPRQELRVALENNTEWSSYFSTVLGLTAFCGGFTSTDSKLGYEANTGLRLIF

SEQ ID NO: 43 - DNA, 4563 bp, Chlamydia muridarum
atgagttccgagaaagataaaaaaaactcctgttctaagttttccttatcggtagtagcagctattctcgcttctatgagtggtttatcgaattgttccgatctttatgccgtaggaagttctgcagaccatcctgcctacttgattcctcaagcggggttattattggatcatattaaggatatattcattggccctaaagatagtcaggataaggggcagtataagttgattattggtgaggctggctctttccaagatagtaatgcagagactcttcctcaaaaggtagagcacagcactttgttttcagttacaacacctataattgtgcaaggaatagatcaacaagatcaggtctcttcgcagggattggtctgtaatttttcaggagatcattcagaggagatttttgagagagaatcctttttagggatcgctttcctagggaatggtagcaaggatggaatcacgttaacagatataaaatcttcgttatctggtgctgccttgtattcttcagatgatcttatttttgaaagaattaagggagatatagagctttcttcttgttcatctttagaaagaggaggagcttgttcagctcaaagtattttaattcatgattgtcaaggattaacggtaaaacattgtgccgcaggggtgaatgttgaaggagttagtgctagcgaccatctcggatttgggggcggggccttctctactacaagttctctttctggagagaagagtttgtatatgcctgcaggcgatattgtggtggctacctgcgatggtcctgtgtgtttcgaaggaaatagtgctcagttagcaaatggtggcgctattgccgcttctggtaaagttctttttgtagctaacgaaaaaaagatttcctttacagacaaccaagctttgtctggaggagctatttctgcatcttctagtatttctttccaaaattgtgctgagcttgtgttcaagagtaatcttgcaaaaggagttaaagataaatgttctttgggaggaggtgctttagcctctttagaatccgtagttttgaaagataatctcggtattacttatgaaaaaaatcagtcctattcggaaggaggggctatttttgggaaggattgtgagatttttgaaaacagggggcctgttgtattcagagataatacagctgctttaggaggcggagctattttggcgcaacaaactgtggcgatttgtggtaataagtctggaatatcttttgaaggaagtaagtctagttttggaggggccattgcttgtggaaatttctcttctgagaataattcttcagctttgggatcaattgatatctctaacaatctaggagatatctcttttcttcggactctgtgtactacttcggatttagggcaaacggattaccaagggggaggggccttattcgctgaaaatatttctctttctgagaatgctggtgcaattactttcaaagacaatattgtgaagacatttgcctcaaatggaaaaatgttgggtggaggggcaattttagcttcaggaaatgttttgattagcaaaaactctggagagatttcttttgtagggaatgctcgagctcctcaggctattccgactcgttcatctgacgaattgtcttttggcgcacaattaactcaaactacttcaggatgttctggaggaggagctctttttggtaaagaggttgccattgttcaaaatgccactgttgtattcgagcaaaatcgcttacagtgtggcgagcaggaaacacatggtggaggcggtgctgtttatggtatggagagtgcctctattattggaaactcttttgtgagattcggaaataattacgctgtagggaatcagatttctggaggagctcttttatccaagaaggtccgtttagctgaaaatacaagggtagatttttctcgaaatatcgctactttctgcggcggggctgttcaagtttctgatggaagttgcgaattgatcaacaatgggtatgtgctattcagagataaccgagggcagacatttggtggggctatttcttgcttgaaaggagatgtgatcatttccggaaataaagatagggttgagtttagagataacattgtgacgcggccttattttgaagaaaatgaagaaaaagttgagacagcagatattaattcagataagcaagaagcagaagagcgctctttattagagaacattgagcagagctttattactgcaactaatcagacctttttcttagaggaagagaaactcccatcagaagcttttatctctgctgaagaactttcaaagagaagagaatgtgctggtggggcgatttttgcaaaacgggtctacattacggataataaagaacctatcttgttttcgcataatttttctgatgtttatgggggagctatttttacgggttctctacaggaaactgataaacaagatgttgtaactcctgaagttgtgatatcaggcaacgatggggatgtcattttttctggaaatgcagctaaacatgataagcatttacctgatacaggtggtggagccatttgtacacagaatttgacgatttcccaaaacaatgggaatgtcttgttcttgaacaattttgcttgttctggtggagcagttcgcatagaggatcatggagaagttcttttagaggcttttgggggagatattattttcaatggaaactcttctttcagagctcaaggatcggatgcgatctattttgctggtaaggactctagaattaaagctttaaatgctactgaaggacatgcgattgtgttccaagatgcattggtgtttgaaaatatagaagaaagaaagtcttcgggactattggtgattaactctcaggaaaatgagggttatacgggatccgtccgatttttaggatctgaaagtaaggttcctcaatggattcatgtgcaacagggaggtcttgagttgctacatggagctattttatgtagttatggggttaaacaagatcctagagctaaaatagtattatctgctggatctaaattgaagattctagattcagagcaagaaaataacgcagaaattggagatcttgaagattctgttaattcagaaaaaacaccatctctttggattgggaagaacgctcaagcaaaagtccctctggttgatatccatactatttctattgatttagcatcattttcttctaaagctcaggaaacccctgaggaagctccacaagtcatcgtccctaagggaagttgtgtccactcgggagagttaagtttggagttggttaatacaacaggaaaaggttatgagaatcatgcgttgttaaaaaatgatactcaggtttctctcatgtctttcaaagaggaaaatgatggatctttagaagatttgagtaagttgtctgtttcggatttacgcattaaagtttctactccagatattgtagaagaaacttatggccatatgggggattggtctgaagctacaattcaagatggggctcttgtcattaattggcatcctactggatataaattagatccgcaaaaagctggttctttggtattcaatgcattatgggaggaagaggctgtattgtctactctaaaaaatgctcggattgcccataaccttaccattcagagaatggaatttgattattctacaaatgcttggggattagcttttagtagctttagagagctatcttcagagaagcttgtttctgttgatggatatagaggctcttatataggggcttctgcaggcattgatactcagttgatggaagattttgttttgggaatcagcacggcttccttcttcgggaaaatgcatagtcagaattttgatgcagagatttctcgacatggttttgttggttcggtctatacaggcttcctagctggggcctggttcttcaaggggcagtacagtcttggcgaaacacataacgatatgacaactcgttacggggttttgggagaatctaatgctacttggaagtctcgaggagtactagcagatgctttagttgaatatcgtagtttagtcggtccagcacgacctaaattttatgctttgcattttaatccttatgtcgaggtatcttatgcatctgcgaagttccctagttttgtagaacaaggaggagaagctcgtgcttttgaagaaacctctttaacaaacattaccgttccctttggtatgaaatttgaactatcttttacaaaaggacagttttcagagactaattctcttggaataggttgtgcatgggaaatgtatcggaaagtcgaaggaagatctgtagagctactagaagctggttttgattgggaaggatctcctatagatctccctaaacaagagctgagagtggctttagaaaacaatacggaatggagttcgtattttagtacagctctaggagtaacagcattttgtggaggattttcttctatggataataaactaggatacgaagcgaatgctggaatgcgtttgattttctag

SEQ ID NO: 44 - protein, 1520 aa, Chlamydia muridarum
MSSEKDKKNSCSKFSLSVVAAILASMSGLSNCSDLYAVGSSADHPAYLIPQAGLLLDHIKDIFIGPKDSQDKGQYKLIIGEAGSFQDSNAETLPQKVEHSTLFSVTTPIIVQGIDQQDQVSSQGLVCNFSGDHSEEIFERESFLGIAFLGNGSKDGITLTDIKSSLSGAALYSSDDLIFERIKGDIELSSCSSLERGGACSAQSILIHDCQGLTVKHCAAGVNVEGVSASDHLGFGGGAFSTTSSLSGEKSLYMPAGDIVVATCDGPVCFEGNSAQLANGGAIAASGKVLFVANEKKISFTDNQALSGGAISASSSISFQNCAELVFKSNLAKGVKDKCSLGGGALASLESVVLKDNLGITYEKNQSYSEGGAIFGKDCEIFENRGPVVFRDNTAALGGGAILAQQTVAICGNKSGISFEGSKSSFGGAIACGNFSSENNSSALGSIDISNNLGDISFLRTLCTTSDLGQTDYQGGGALFAENISLSENAGAITFKDNIVKTFASNGKMLGGGAILASGNVLISKNSGEISFVGNARAPQAIPTRSSDELSFGAQLTQTTSGCSGGGALFGKEVAIVQNATVVFEQNRLQCGEQETHGGGGAVYGMESASIIGNSFVRFGNNYAVGNQISGGALLSKKVRLAENTRVDFSRNIATFCGGAVQVSDGSCELINNGYVLFRDNRGQTFGGAISCLKGDVIISGNKDRVEFRDNIVTRPYFEENEEKVETADINSDKQEAEERSLLENIEQSFITATNQTFFLEEEKLPSEAFISAEELSKRRECAGGAIFAKRVYITDNKEPILFSHNFSDVYGGAIFTGSLQETDKQDVVTPEVVISGNDGDVIFSGNAAKHDKHLPDTGGGAICTQNLTISQNNGNVLFLNNFACSGGAVRIEDHGEVLLEAFGGDIIFNGNSSFRAQGSDAIYFAGKDSRIKALNATEGHAIVFQDALVFENIEERKSSGLLVINSQENEGYTGSVRFLGSESKVPQWIHVQQGGLELLHGAILCSYGVKQDPRAKIVLSAGSKLKILDSEQENNAEIGDLEDSVNSEKTPSLWIGKNAQAKVPLVDIHTISIDLASFSSKAQETPEEAPQVIVPKGSCVHSGELSLELVNTTGKGYENHALLKNDTQVSLMSFKEENDGSLEDLSKLSVSDLRIKVSTPDIVEETYGHMGDWSEATIQDGALVINWHPTGYKLDPQKAGSLVFNALWEEEAVLSTLKNARIAHNLTIQRMEFDYSTNAWGLAFSSFRELSSEKLVSVDGYRGSYIGASAGIDTQLMEDFVLGISTASFFGKMHSQNFDAEISRHGFVGSVYTGFLAGAWFFKGQYSLGETHNDMTTRYGVLGESNATWKSRGVLADALVEYRSLVGPARPKFYALHFNPYVEVSYASAKFPSFVEQGGEARAFEETSLTNITVPFGMKFELSFTKGQFSETNSLGIGCAWEMYRKVEGRSVELLEAGFDWEGSPIDLPKQELRVALENNTEWSSYFSTALGVTAFCGGFSSMDNKLGYEANAGMRLIF

SEQ ID NO: 45 - DNA, 4614 bp, Chlamydia psitacci
atggtcgcaaaaaaggtatcaagatttccaaaatctacattttcccattccgtagttttagcaatattagtttctactgggatgactgctaataatcatagattatatggttatgagacagtttcagaggcgtttttgagtgattcttctttgaaaacccaattagaaacgacttctgcgggtgtttttagaaaagtaaaatctactgatacacaagaggttcagaaagaaaataaagaagaaaatacgcctgtagagacttcttttatagagaatgcttcttcatgttctgttgctattttaggctcagaatgcggtcaaagacagcatttagtcaatgccagtactttgtttgagatatcggattctttatcatggaagagtatagatggcgagttgtctaaaagctccaagaagtctgctacagccgaagatgcagagagaaaatatcttgtggatgattccagtcaaggtttagctttttgttataaaaacccatcagattgtgttgtggatgaaactacgcctggattcttaggtgttgctcttgtaggagtgggatctacatcaggactatctttttctaatttaaagtcgctctcagcaggatctgcggtctattctgatgaagatgttgtttttgaacaccttaaagaaaaactgttttttgaaggttgtgagtctcaagcaggtggtggagctgtttcaggacgtagtattgctataaatggttgccatgacgtttctgcagtgtcttgtaagaccgatttagatcttgcatcttctgaagtcgtagatttttccaaaggtggaggtgcttttaacgcgcataaggtgcatggtgaagcacataaatccagattttttactggagaaatcatctttacagccaattctgggaatgttttgctagatggtaatcatgcagacaaggcgaacggtggagttgtagcctgtggagcatttgtttgttctgtaaatcgtggagatatccgctacacaagtaaccgcgctctatctggaggcgctgtatctgcttttaagtctattgattttgttggaaacgtaggattgatagagtttgtagataaccaggctttaatttctcctgaaagttctttatttttaggtggtggggccttagcttctggagagagaattagtttcttaaacaatggaggcatccattgttgcaaaaacacctctaaatcctctggaggagctcttttatctagggatgtaagaattgtagagaacatcggaaattctttgtttaaggaaaactctgctcaagtcgtaggtggagccattagttctcaaaatcaagtagaggttggtcagaattttggaaatatcacttttgaaggtaatacttccaagatgggtggtggagctattcactgtttatctgctcagcaaccttatacgagttctgaagaagctctggaaggatctggggatatcaagattgttgataattcgggggctgtaaattttgcatctaatgagaacctattggaatctcaagagacacatagtcatattggtggtggtgctctatacggatcaaatgttttagtttcaggcaatattggagaggttactttttctaagaataccgctggtcaatgtgaatccgacagtacctgtataggtggtggagcggtttttgctaatgaggctgttagaatagtagataactcaggagcgattactttctcttataataaagggacaattcttccatttcctaaagttgctgcaagttctgaaggggaaagtgctccagaagctcctaaagagtcatctcctgtagatttaggggttcgcggcggtggagcaatttttgccaagcgtatagagatagcagataactctggtgtactatctttctcagataatttcatgaaaattagagataataaggcacaaaaagagaatcctctaggcggtggtgctttatttgggatagatgaagtcggtttgaaaaataataaagaacttgcattcactaataaccatgtctctggtgagaatagtagcggtggtgctgtcctatctaaagttgtcactattgctgataatggaaaagtacaatttttccgcaattattcgaatttccttggtggtgccgtttgttctctaggagatgctctaaacattaaaaataatgaatcttcagtatcttttattggcaacagaactgtgactgctggtggagcgcttgctagtgctgcaggtgatgtttctatttctaaaaaccttgggaaagtagaatttaaggataatttagtttttggcgattctcgtgtagataatcttgaagaaggtcaactcaacactacaggacatcatagtggcggcggtgccatttttgctaaagcttcagtggttattcgtgaaaataaagatcaggtgcttttctcagggaattcttcaggatgtttcggtggtgcgattttaacaggttctttaaccccagaagatcaagagcgttttgcttctaaggtagtgaatgataatactaaagtcgttattacagagaacattggagacgtagtattttcaggaaatagcactacggcttcaaaacatcctgagcataatttgttcggtggtggtgctatctatacccaagacttaattatcaataaaaatgcaggttctgtagctttttataataactacgctcctacaggtggtgctgtccgtattagtgaaaagggaactgtgattttagaggctctaggaggagatattgttttccaaggaaatagaaattctgaagatatctctaatggattatattttgccggaaaagagtcgaaattagttgaggtatctgcttctggggaaaaaacggttaatttttcagatgccattatctttgaagatttaaccttaagacaaggcctagaaggtcgtgaggatattttaaatgatcctacattagtattgaattctaaggctaaagatgattctgaagtttctcattctggaaacattcgctttgcctatgcgacatctaagattcctcaagtcgctgtattagaatcaggaactcttattttatctgataatgctgagttgtggttgtgtggcttaaaacaagagaaaggtagtgagatcttgctctcagcaggaactgtattacgtattttcgatcctaatgctaagcctgaagaaaagcctgaaagtccttctgcaagatcttactatagtgcttatgattctgctagaaatcctgaagagaagactttggcagacatcagtgttattggtgtagatctagcttcttttgttgctagtgaggatgaagctgctcctttacctccacagattatcgttcctaagggcacaacaatcggttcgggatctttagacttgaatcttgtggattctgcaggtgttggttatgaaaaccatgccttattaaataaagagactgatatcacattgctttcatttaggagtgcctcagcagtctcggatgttcctgatttagaccatgctttggaagagttacgtattaacgtttctgttcctaaaattacggacgacacttatgggcatatgggaaaatggtcagatcctcaggttgttaacggtaaattaacgatcaactggaagcctaccagctataagttaaatcctgaaaaaggaggctctatcgtattgaacactttatggggacaatgcggagatttgcgcgccttaaaacaacagcatttatctcataatattactgcacaaagaatggaattagatttctcaacaaacatttggggatctggaatgggaacattctccaattgtgcaacgattgctggagtggacggctttactcatcgtgctggcggctatgctttaggtttagatacacagttgatagaagatttcttgataggaggaagctttgcgcagttctttggttacactgatagtcagtcattttcatcacgtagtgaccaaagtggttacttaggtacgggatatgtcggtatctttgcgggttcttggttattcaaggggatgtttatctatagtgatattcaaaacgacttgaatacaacatatcctacaccaaacattggtagatctaaaggatcgtggaatagccgcggtatcttagcagatgctcatgtggattatcgctatattgtgaattcacgtaggtttatctcatcgattgtttcggctgtggtacctttcgtagaagctgaatatgtttacattgatcttcctacatttgcggaagtaggtagtgaagtgagaacatttgctgaagggcatttacaaaatatagcgattccttttgggattactttggagcataactattctcgagggcagcgttcagaagtgaatagcttaagtttctcctatgctttagatgtctatcgtaaagcacctacagtgcttatcaatttgcctgcagcttcttattcttgggagggggtaggttctgatctttctagaaagtttatgaaagcacagtttagtaatgatacggagtggagttcctacttctctactttcttagggtttacctatgaatggagagaacacacggtatcttatgatgtgaatggaggtatacgtttgatattctag

SEQ ID NO: 46 - protein, 1537 aa, Chlamydia psitacci
MVAKKVSRFPKSTFSHSVVLAILVSTGMTANNHRLYGYETVSEAFLSDSSLKTQLETTSAGVFRKVKSTDTQEVQKENKEENTPVETSFIENASSCSVAILGSECGQRQHLVNASTLFEISDSLSWKSIDGELSKSSKKSATAEDAERKYLVDDSSQGLAFCYKNPSDCVVDETTPGFLGVALVGVGSTSGLSFSNLKSLSAGSAVYSDEDVVFEHLKEKLFFEGCESQAGGGAVSGRSIAINGCHDVSAVSCKTDLDLASSEVVDFSKGGGAFNAHKVHGEAHKSRFFTGEIIFTANSGNVLLDGNHADKANGGVVACGAFVCSVNRGDIRYTSNRALSGGAVSAFKSIDFVGNVGLIEFVDNQALISPESSLFLGGGALASGERISFLNNGGIHCCKNTSKSSGGALLSRDVRIVENIGNSLFKENSAQVVGGAISSQNQVEVGQNFGNITFEGNTSKMGGGAIHCLSAQQPYTSSEEALEGSGDIKIVDNSGAVNFASNENLLESQETHSHIGGGALYGSNVLVSGNIGEVTFSKNTAGQCESDSTCIGGGAVFANEAVRIVDNSGAITFSYNKGTILPFPKVAASSEGESAPEAPKESSPVDLGVRGGGAIFAKRIEIADNSGVLSFSDNFMKIRDNKAQKENPLGGGALFGIDEVGLKNNKELAFTNNHVSGENSSGGAVLSKVVTIADNGKVQFFRNYSNFLGGAVCSLGDALNIKNNESSVSFIGNRTVTAGGALASAAGDVSISKNLGKVEFKDNLVFGDSRVDNLEEGQLNTTGHHSGGGAIFAKASVVIRENKDQVLFSGNSSGCFGGAILTGSLTPEDQERFASKVVNDNTKVVITENIGDVVFSGNSTTASKHPEHNLFGGGAIYTQDLIINKNAGSVAFYNNYAPTGGAVRISEKGTVILEALGGDIVFQGNRNSEDISNGLYFAGKESKLVEVSASGEKTVNFSDAIIFEDLTLRQGLEGREDILNDPTLVLNSKAKDDSEVSHSGNIRFAYATSKIPQVAVLESGTLILSDNAELWLCGLKQEKGSEILLSAGTVLRIFDPNAKPEEKPESPSARSYYSAYDSARNPEEKTLADISVIGVDLASFVASEDEAAPLPPQIIVPKGTTIGSGSLDLNLVDSAGVGYENHALLNKETDITLLSFRSASAVSDVPDLDHALEELRINVSVPKITDDTYGHMGKWSDPQVVNGKLTINWKPTSYKLNPEKGGSIVLNTLWGQCGDLRALKQQHLSHNITAQRMELDFSTNIWGSGMGTFSNCATIAGVDGFTHRAGGYALGLDTQLIEDFLIGGSFAQFFGYTDSQSFSSRSDQSGYLGTGYVGIFAGSWLFKGMFIYSDIQNDLNTTYPTPNIGRSKGSWNSRGILADAHVDYRYIVNSRRFISSIVSAVVPFVEAEYVYIDLPTFAEVGSEVRTFAEGHLQNIAIPFGITLEHNYSRGQRSEVNSLSFSYALDVYRKAPTVLINLPAASYSWEGVGSDLSRKFMKAQFSNDTEWSSYFSTFLGFTYEWREHTVSYDVNGGIRLIF

SEQ ID NO: 47 - DNA, 1185 bp, Chlamydia trachomatis
atgaaaaaactcttgaaatcggtattagtgtttgccgctttgagttctgcttcctccttgcaagctctgcctgtggggaatcctgctgaaccaagccttatgatcgacggaattctatgggaaggtttcggcggagatccttgcgatccttgcaccacttggtgtgacgctatcagcatgcgtatgggttactatggtgactttgttttcgaccgtgttttgcaaacagatgtgaataaagaattccaaatgggtgccaagcctacaactgctacaggcaatgctgcagctccatccacttgtacagcaagagagaatcctgcttacggccgacatatgcaggatgctgagatgtttacaaatgctgcttacatggcattgaatatttgggatcgttttgatgtattctgtacattaggagccaccagtggatatcttaaaggaaattcagcatctttcaacttagttggcttattcggagataatgagaaccatgctacagtttcagatagtaagcttgtaccaaatatgagcttagatcaatctgttgttgagttgtatacagatactacttttgcttggagtgctggagctcgtgcagctttgtgggaatgtggatgcgcgactttaggcgcttctttccaatacgctcaatccaagcctaaagtcgaagaattaaacgttctctgtaacgcagctgagtttactatcaataagcctaaaggatatgtagggcaagaattccctcttgatcttaaagcaggaacagatggtgtgacaggaactaaggatgcctctattgattaccatgaatggcaagcaagtttagctctctcttacagactgaatatgttcactccctacattggagttaaatggtctcgagcaagttttgatgcagacacgattcgtattgctcagccgaagtcagctacaactgtctttgatgttaccactctgaacccaactattgctggagctggcgatgtgaaagctagcgcagagggtcagctcggagataccatgcaaatcgtttccttgcaattgaacaagatgaaatctagaaaatcttgcggtattgcagtaggaacaactattgtggatgcagacaaatacgcagttacagttgagactcgcttgatcgatgagagagctgctcacgtaaatgcacaattccgcttctaa

SEQ ID NO: 48 - protein, 394 aa, Chlamydia trachomatis
MKKLLKSVLVFAALSSASSLQALPVGNPAEPSLMIDGILWEGFGGDPCDPCTTWCDAISMRMGYYGDFVFDRVLQTDVNKEFQMGAKPTTATGNAAAPSTCTARENPAYGRHMQDAEMFTNAAYMALNIWDRFDVFCTLGATSGYLKGNSASFNLVGLFGDNENHATVSDSKLVPNMSLDQSVVELYTDTTFAWSAGARAALWECGCATLGASFQYAQSKPKVEELNVLCNAAEFTINKPKGYVGQEFPLDLKAGTDGVTGTKDASIDYHEWQASLALSYRLNMFTPYIGVKWSRASFDADTIRIAQPKSATTVFDVTTLNPTIAGAGDVKASAEGQLGDTMQIVSLQLNKMKSRKSCGIAVGTTIVDADKYAVTVETRLIDERAAHVNAQFRF

SEQ ID NO: 49 - DNA, 1194 bp, Chlamydia trachomatis
atgaaaaaactcttgaaatcggtattagtatttgccgctttgagttctgcttcctccttgcaagctctgcctgtggggaatcctgctgaaccaagccttatgatcgacggaattctgtgggaaggtttcggtggagatccttgcgatccttgcaccacttggtgtgacgctatcagcatgcgtatgggttactatggtgactttgttttcgaccgtgttttgaaaacagatgtgaataaagaatttcagatgggagcggcgcctactaccagcgatgtagcaggcttacaaaacgatccaacaacaaatgttgctcgtccaaatcccgcttatggcaaacacatgcaagatgctgaaatgtttacgaacgctgcttacatggcattaaatatctgggatcgttttgatgtattttgtacattgggagcaactaccggttatttaaaaggaaactccgcttccttcaacttagttggattattcggaacaaaaacacaagcttctagctttaatacagcgaatctttttcctaacactgctttgaatcaagctgtggttgagctttatacagacactacctttgcttggagcgtaggtgctcgtgcagctctctgggaatgtgggtgtgcaacgttaggagcttctttccaatatgctcaatctaaacctaaagtagaagagttaaatgttctttgtaatgcatccgaatttactattaataagccgaaaggatatgttggggcggaatttccacttgatattaccgcaggaacagaagctgcgacagggactaaggatgcctctattgactaccatgagtggcaagcaagtttagccctttcttacagattaaatatgttcactccttacattggagttaaatggtctagagtaagttttgatgccgacacgatccgtatcgctcagcctaaattggctgaagcaatcttggatgtcactactctaaacccgaccatcgctggtaaaggaactgtggtcgcttccggaagcgaaaacgacctggctgatacaatgcaaatcgtttccttgcagttgaacaagatgaaatctagaaaatcttgcggtattgcagtaggaacgactattgtagatgcagacaaatacgcagttacagttgagactcgcttgatcgatgagagagcagctcacgtaaatgcacaattccgcttctaa

SEQ ID NO: 50 - protein, 397 aa, Chlamydia trachomatis
MKKLLKSVLVFAALSSASSLQALPVGNPAEPSLMIDGILWEGFGGDPCDPCTTWCDAISMRMGYYGDFVFDRVLKTDVNKEFQMGAAPTTSDVAGLQNDPTTNVARPNPAYGKHMQDAEMFTNAAYMALNIWDRFDVFCTLGATTGYLKGNSASFNLVGLFGTKTQASSFNTANLFPNTALNQAVVELYTDTTFAWSVGARAALWECGCATLGASFQYAQSKPKVEELNVLCNASEFTINKPKGYVGAEFPLDITAGTEAATGTKDASIDYHEWQASLALSYRLNMFTPYIGVKWSRVSFDADTIRIAQPKLAEAILDVTTLNPTIAGKGTVVASGSENDLADTMQIVSLQLNKMKSRKSCGIAVGTTIVDADKYAVTVETRLIDERAAHVNAQFRF

SEQ ID NO: 51 - DNA, 1194 bp, Chlamydia trachomatis
atgaaaaaactcttgaaatcggtattagtatttgccgctttgagttctgcttcctccttgcaagctctgcctgtggggaatcctgctgaaccaagccttatgatcgacggaattctgtgggaaggttttggcggagatccttgcgatccttgcgccacttggtgtgacgctatcagcatgcgtgttggttactacggagactttgttttcgaccgtgttttgaaaactgatgtgaataaagaatttcagatgggagcggcgcctactaccaacgatgcagcagacttacaaaacgatccaaaaacaaatgttgctcgtccaaatcccgcttatggcaaacacatgcaagatgctgaaatgtttacgaacgctgcttacatggcattaaatatctgggatcgttttgatgtattttgtacattgggagcaactaccggttatttaaaaggaaactccgcttccttcaacttagttggattattcggaacaaaaacaaaatcttctgattttaatacagcgaagcttgttcctaacattgctttgaatcgagctgtggttgagctttatacagacactacctttgcttggagcgtaggtgctcgtgcagctctctgggaatgtgggtgtgcaacgttaggagcttctttccaatatgctcaatctaaacctaaagtagaagagttaaatgttctttgtaatgcatccgaatttactattaataagccgaaaggatatgttggggcggaatttccacttgatattaccgcaggaacagaagctgcgacagggactaaggatgcctctattgactaccatgagtggcaagcaagtttagccctttcttacagactaaatatgttcactccttacattggagttaaatggtctagagtaagttttgatgccgacacgatccgtatcgctcagcctaaattggctgaagcaatcttggatgtcactactctaaacccgaccatcgctggtaaaggaactgtggtcgcttccggaagcgataacgacctggctgatacaatgcaaatcgtttccttgcagttgaacaagatgaaatctagaaaatcttgcggtattgcagtaggaacgactattgtagatgcagacaaatacgcagttacagttgagactcgcttgatcgatgagagagcagctcacgtaaatgcacaattccgcttctaa

SEQ ID NO: 52 - protein, 397 aa, Chlamydia trachomatis
MKKLLKSVLVFAALSSASSLQALPVGNPAEPSLMIDGILWEGFGGDPCDPCATWCDAISMRVGYYGDFVFDRVLKTDVNKEFQMGAAPTTNDAADLQNDPKTNVARPNPAYGKHMQDAEMFTNAAYMALNIWDRFDVFCTLGATTGYLKGNSASFNLVGLFGTKTKSSDFNTAKLVPNIALNRAVVELYTDTTFAWSVGARAALWECGCATLGASFQYAQSKPKVEELNVLCNASEFTINKPKGYVGAEFPLDITAGTEAATGTKDASIDYHEWQASLALSYRLNMFTPYIGVKWSRVSFDADTIRIAQPKLAEAILDVTTLNPTIAGKGTVVASGSDNDLADTMQIVSLQLNKMKSRKSCGIAVGTTIVDADKYAVTVETRLIDERAAHVNAQFRF

SEQ ID NO: 53 - DNA, 1182 bp, Chlamydia trachomatis
atgaaaaaactcttgaaatcggtattagtatttgccgctttgagttctgcttcctccttgcaagctctgcctgtggggaatcctgctgaaccaagccttatgatcgacggaattctgtgggaaggtttcggcggagatccttgcgatccttgcaccacttggtgtgacgctatcagcatgcgtatgggttactatggtgactttgttttcgaccgtgttttgaaaacagatgtgaataaagaattccaaatgggtgacaagcctacaagtactacaggcaatgctacagctccaaccactcttacagcaagagagaatcctgcttacggccgacatatgcaggatgctgagatgtttacaaatgccgcttgcatggcattgaatatttgggatcgctttgatgtattctgtacactaggagcctctagcggataccttaaaggaaactctgcttctttcaatttagttggattgtttggagataatgaaaatcaaagcacggtcaaaacgaattctgtaccaaatatgagcttagatcaatctgttgttgaactttacacagatactgccttctcttggagcgtgggcgctcgagcagctttgtgggagtgcggatgtgcgactttaggggcttctttccaatacgctcaatctaaacctaaagtcgaagaattaaacgttctctgtaacgcagctgagtttactatcaataagcctaaaggatatgtagggcaagaattccctcttgcactcatagcaggaactgatgcagcgacgggcactaaagatgcctctattgattaccatgagtggcaagcaagtttagctctctcttacagattgaatatgttcactccctacattggagttaaatggtctcgagcaagttttgatgccgatacgattcgtatagcccagccaaaatcagctacagctatctttgatactaccacgcttaacccaactattgctggagctggcgatgtgaaagctagcgcagagggtcagctcggagataccatgcaaatcgtctccttgcaattgaacaagatgaaatctagaaaatcttgcggtattgcagtaggaacgactattgtagatgcagacaaatacgcagttacagttgagactcgcttgatcgatgagagagctgctcacgtaaatgcacaattccgcttctaa

SEQ ID NO: 54 - protein, 393 aa, Chlamydia trachomatis
MKKLLKSVLVFAALSSASSLQALPVGNPAEPSLMIDGILWEGFGGDPCDPCTTWCDAISMRMGYYGDFVFDRVLKTDVNKEFQMGDKPTSTTGNATAPTTLTARENPAYGRHMQDAEMFTNAACMALNIWDRFDVFCTLGASSGYLKGNSASFNLVGLFGDNENQSTVKTNSVPNMSLDQSVVELYTDTAFSWSVGARAALWECGCATLGASFQYAQSKPKVEELNVLCNAAEFTINKPKGYVGQEFPLALIAGTDAATGTKDASIDYHEWQASLALSYRLNMFTPYIGVKWSRASFDADTIRIAQPKSATAIFDTTTLNPTIAGAGDVKASAEGQLGDTMQIVSLQLNKMKSRKSCGIAVGTTIVDADKYAVTVETRLIDERAAHVNAQFRF

SEQ ID NO: 55 - DNA, 1179 bp, Chlamydia trachomatis
atgaaaaaactcttgaaatcggtattagtatttgccgctttgagttctgcttcctccttgcaagctctgcctgtggggaatcctgctgaaccaagccttatgatcgacggaattctgtgggaaggtttcggcggagatccttgcgatccttgcgccacttggtgtgacgctatcagcatgcgtgttggttactacggagactttgttttcgaccgtgttttgaaaactgatgtgaataaagaatttcagatgggtgccaagcctacaactgatacaggcaatagtgcagctccatccactcttacagcaagagagaatcctgcttacggccgacatatgcaggatgctgagatgtttacaaatgccgcttgcatggcattgaatatttgggatcgttttgatgtattctgtacattaggagccaccagtggatatcttaaaggaaactctgcttctttcaatttagttggattgtttggagataatgaaaatcaaaaaacggtcaaagcggagtctgtaccaaatatgagctttgatcaatctgttgttgagttgtatacagatactacttttgcgtggagcgtcggcgctcgcgcagctttgtgggaatgtggatgtgcaactttaggagcttcattccaatatgctcaatctaaacctaaagtagaagaattaaacgttctctgcaatgcagcagagtttactattaataaacctaaagggtatgtaggtaaggagtttcctcttgatcttacagcaggaacagatgctgcgacaggaactaaggatgcctctattgattaccatgaatggcaagcaagtttagctctctcttacagactgaatatgttcactccctacattggagttaaatggtctcgagcaagctttgatgccgatacgattcgtatagcccagccaaaatcagctacagctatttttgatactaccacgcttaacccaactattgctggagctggcgatgtgaaaactggcgcagagggtcagctcggagacacaatgcaaatcgtttccttgcaattgaacaagatgaaatctagaaaatcttgcggtattgcagtaggaacaactattgtggatgcagacaaatacgcagttacagttgagactcgcttgatcgatgagagagcagctcacgtaaatgcacaattccgcttc

SEQ ID NO: 56 - protein, 393 aa, Chlamydia trachomatis
MKKLLKSVLVFAALSSASSLQALPVGNPAEPSLMIDGILWEGFGGDPCDPCATWCDAISMRVGYYGDFVFDRVLKTDVNKEFQMGAKPTTDTGNSAAPSTLTARENPAYGRHMQDAEMFTNAACMALNIWDRFDVFCTLGATSGYLKGNSASFNLVGLFGDNENQKTVKAESVPNMSFDQSVVELYTDTTFAWSVGARAALWECGCATLGASFQYAQSKPKVEELNVLCNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLNKMKSRKSCGIAVGTTIVDADKYAVTVETRLIDERAAHVNAQFRF

SEQ ID NO: 57 - DNA, 1944 bp, Chlamydia trachomatis
atggaatcaggaccagaatcagtttcttctaatcagagctcgatgaatccaattattaatgggcaaatcgcttctaattcggagaccaaagagtccacgaaggagtcagaagcgagtccttcagcatcgtcctctgtaagcagctggagttttttatcctcagcaaagcatgcattaatctctcttcgtgatgccatcttgaataaaaattctagtccaacagactctctctctcaattagaggcctctacttctacctctacggttacacgtgtagctgcgcgagattataatgaggctaaatcgaattttgatacggcgaaaagtggattagagaacgctacgacacttgctgaatacgagacgaaaatggctgatttaatggcagctctccaagatatggagcgtttggctaaacagaaggctgaagttacaagaattaaagaagctcttcaagagaaacaagaggttattgataagctcaatcagttagttaaacttgaaaaacagaatcagactttaaaggaaactttaacaaccacagactctgcagatcagattccagcgattaatagtcagttagagatcaacaaaaattctgcagatcaaattatcaaagatctggaaggacaaaacataagttatgaagctgttctcactaacgcaggagaggttatcaaagcttcttctgaagcgggaattaagttaggacaagctttgcagtctattgtggatgctggggatcaaagccaggctgcagttcttcaagcacagcaaaataatagcccagataatatcgcagccacgaagaaattaattgatgctgctgaaacgaaggtaaacgagttaaaacaagagcatacagggctaacggactcgcctttagtgaaaaaagctgaggagcagattagtcaagcacaaaaagatattcaagagatcaaacctagtggttcggatattcctatcgttggtccgagtgggtcagctgcttccgcaggaagtgcggtaggagcgttgaaatcctctaacaattcaggaagaatttccttgttgcttgatgatgtagacaatgaaatggcagcgattgcaatgcaaggttttcgatctatgatcgaacaatttaatgtaaacaatcctgcaacagctaaagagctacaagctatggaggctcagctgactgcgatgtcagatcaactggttggtgcggatggcgagctcccagccgaaatacaagcaatcaaagatgctcttgcgcaagctttgaaacaaccatcaacagatggtttagctacagctatgggacaagtggcttttgcagctgccaaggttggaggaggctccgcaggaacagctggcactgtccagatgaatgtaaaacagctttacaagacagcgttttcttcgacttcttccagctcttatgcagcagcactttccgatggatattctgcttacaaaacactgaactctttatattccgaaagcagaagcggcgtgcagtcagctattagtcaaactgcaaatcccgcgctttccagaagcgtttctcgttctggcatagaaagtcaaggacgcagtgcagatgctagccaaagagcagcagaaactattgtcagagatagccaaacgttaggtgatgtatatagccgcttacaggttctggattctttgatgtctacgattgtgagcaatccgcaagtaaatcaagaagagattatgcagaagctcacggcatctattagcaaagctccacaatttgggtatcctgctgttcagaattctgcggatagcttgcagaagtttgctgcgcaattggaaagagagtttgttgatggggaacgtagtctcgcagaatctcgagagaatgcgtttagaaaacagcccgctttcattcaacaggtgttggtaaacattgcttctctattctctggttatctttcttaa

SEQ ID NO: 58 - protein, 647 aa, Chlamydia trachomatis
MESGPESVSSNQSSMNPIINGQIASNSETKESTKESEASPSASSSVSSWSFLSSAKHALISLRDAILNKNSSPTDSLSQLEASTSTSTVTRVAARDYNEAKSNFDTAKSGLENATTLAEYETKMADLMAALQDMERLAKQKAEVTRIKEALQEKQEVIDKLNQLVKLEKQNQTLKETLTTTDSADQIPAINSQLEINKNSADQIIKDLEGQNISYEAVLTNAGEVIKASSEAGIKLGQALQSIVDAGDQSQAAVLQAQQNNSPDNIAATKKLIDAAETKVNELKQEHTGLTDSPLVKKAEEQISQAQKDIQEIKPSGSDIPIVGPSGSAASAGSAVGALKSSNNSGRISLLLDDVDNEMAAIAMQGFRSMIEQFNVNNPATAKELQAMEAQLTAMSDQLVGADGELPAEIQAIKDALAQALKQPSTDGLATAMGQVAFAAAKVGGGSAGTAGTVQMNVKQLYKTAFSSTSSSSYAAALSDGYSAYKTLNSLYSESRSGVQSAISQTANPALSRSVSRSGIESQGRSADASQRAAETIVRDSQTLGDVYSRLQVLDSLMSTIVSNPQVNQEEIMQKLTASISKAPQFGYPAVQNSADSLQKFAAQLEREFVDGERSLAESRENAFRKQPAFIQQVLVNIASLFSGYLS

SEQ ID NO: 59 - DNA, 1911 bp, Chlamydia psitacci
atggttaatcctgtcggccctatagatgaatcaaaaaacattgctcctgcagacttatctactttaggtatgcaggcgagcgcagcaaatcgtagctcagaagctcaatcgataaccggaattgcaggcaagtccgggtcatcgcagccttctgtggaaactgtaggacgattgagctttttgagctctgctcggaaaagtttagcaagtcttttcgataagatttcctcgttcttttcagggaaaacgactcctcaaacttttgatgaagctaagacgcaagcagagagtgcgaaaactgcgctgcagagtgcgactacttatgatcagttcaagaccgctttacagcagctgcaagatgctgtgaaacagatggagcaattagctactactgatgcagaaaaagctacagttgctacatggaaaacggctcttgaggcgcagaagagtacgctggatacacttaaccagttgggtgctattcttacagagaaccagaagcttcttgaggcaataaagacgacctcgtctatggatcagattatgggagctgccggacaagtagaaaccaataaaacaactgctgaggagttaattaaacagttgaaggaagctggggttagctatcctgtgatagatgaccttgagaagcaaattacaacctcaggaactcaggttactgaattagcagatgctatatcggaagcttatgctgcggggaaaaacagtaccgcggctgtggggcaagcacaggcaaataacagccccgcaaatatagaagcttccaaacaaactattgcaaatgcacaaaaagtcatagaagacgctcttaaacttgctccagattctccgatactcaaagctgctttgaaagaacaacaacaggcagcaaaagatatcctcaatgtgaaacctagtggtggtagtgatgtgcctatcggtggtcctggagctcctggtagtgtggggacttctcaaaatcgcggtgctaccttaggggaagttcgcgtatcgatgttattgactgatgttgataatgaaaccgcagcgatcattatgcaaggtttcagaaatatgatcgataacttccatgatcaaaactctgattttacagcgcctttagaagagattatgaatcaagtaaccgacttatcaacgcagatcaatcctgcagatgcggaagctacagcacaactacaagaaatacaacaaaccatacaagatgcccttcaagggactgccggtcaagacggcatgatcaatgctttaggagctataacaacagcagcttcaatttctacaggagctcctatcgcttctgcaaatcaaggtggatcagctgtaaagcagctttacaaaacaggatctactgctgcgagttctaaatcttacgcggattccttatctgcagggtatggggcatatcaatctttaaatgatgtgtactcacgtagtagtgcatctaaccgtgaggttttagatcgtacatcgactccagcattaacgcagacagtttctagaacagaaactcggcctcgtgataatgataacgcagctcagcgttttgcaagaactatagctgctaatagtaatactcttggggatgtttatgcatccgtaggtgtattgcaaacattgctaggtgtattacaaaataatccccaagcgaatgaagaagaaatcaaacagaagctcacttctgaggttacgaaagctccgcagtcaggttatcctcatgtacagctttctaacgactctacgaagaagttcattgctcaactcgagaatgaatttgttcagggatcgaaaagacttgccgaagcaaaagaagctgcgtttgagaaacagcctttgttcatccagcaggtattagtgaacgtagcatctctgttctcgggatacctacagtaa

SEQ ID NO: 60 - protein, 636 aa, Chlamydia psitacci
MVNPVGPIDESKNIAPADLSTLGMQASAANRSSEAQSITGIAGKSGSSQPSVETVGRLSFLSSARKSLASLFDKISSFFSGKTTPQTFDEAKTQAESAKTALQSATTYDQFKTALQQLQDAVKQMEQLATTDAEKATVATWKTALEAQKSTLDTLNQLGAILTENQKLLEAIKTTSSMDQIMGAAGQVETNKTTAEELIKQLKEAGVSYPVIDDLEKQITTSGTQVTELADAISEAYAAGKNSTAAVGQAQANNSPANIEASKQTIANAQKVIEDALKLAPDSPILKAALKEQQQAAKDILNVKPSGGSDVPIGGPGAPGSVGTSQNRGATLGEVRVSMLLTDVDNETAAIIMQGFRNMIDNFHDQNSDFTAPLEEIMNQVTDLSTQINPADAEATAQLQEIQQTIQDALQGTAGQDGMINALGAITTAASISTGAPIASANQGGSAVKQLYKTGSTAASSKSYADSLSAGYGAYQSLNDVYSRSSASNREVLDRTSTPALTQTVSRTETRPRDNDNAAQRFARTIAANSNTLGDVYASVGVLQTLLGVLQNNPQANEEEIKQKLTSEVTKAPQSGYPHVQLSNDSTKKFIAQLENEFVQGSKRLAEAKEAAFEKQPLFIQQVLVNVASLFSGYLQ

SEQ ID NO: 61 - DNA, 1956 bp, Chlamydia pneumoniae
atggttaatcctattggtccaggtcctatagacgaaacagaacgcacacctcccgcagatctttctgctcaaggattggaggcgagtgcagcaaataagagtgcggaagctcaaagaatagcaggtgcggaagctaagcctaaagaatctaagaccgattctgtagagcgatggagcatcttgcgttctgcagtgaatgctctcatgagtctggcagataagctgggtattgcttctagtaacagctcgtcttctactagcagatctgcagacgtggactcaacgacagcgaccgcacctacgcctcctccacccacgtttgatgattataagactcaagcgcaaacagcttacgatactatctttacctcaacatcactagctgacatacaggctgctttggtgagcctccaggatgctgtcactaatataaaggatacagcggctactgatgaggaaaccgcaatcgctgcggagtgggaaactaagaatgccgatgcagttaaagttggcgcgcaaattacagaattagcgaaatatgcttcggataaccaagcgattcttgactctttaggtaaactgacttccttcgacctcttacaggctgctcttctccaatctgtagcaaacaataacaaagcagctgagcttcttaaagagatgcaagataacccagtagtcccagggaaaacgcctgcaattgctcaatctttagttgatcagacagatgctacagcgacacagatagagaaagatggaaatgcgattagggatgcatattttgcaggacagaacgctagtggagctgtagaaaatgctaaatctaataacagtataagcaacatagattcagctaaagcagcaatcgctactgctaagacacaaatagctgaagctcagaaaaagttccccgactctccaattcttcaagaagcggaacaaatggtaatacaggctgagaaagatcttaaaaatatcaaacctgcagatggttctgatgttccaaatccaggaactacagttggaggctccaagcaacaaggaagtagtattggtagtattcgtgtttccatgctgttagatgatgctgaaaatgagaccgcttccattttgatgtctgggtttcgtcagatgattcacatgttcaatacggaaaatcctgattctcaagctgcccaacaggagctcgcagcacaagctagagcagcgaaagccgctggagatgacagtgctgctgcagcgctggcagatgctcagaaagctttagaagcggctctaggtaaagctgggcaacaacagggcatactcaatgctttaggacagatcgcttctgctgctgttgtgagcgcaggagttcctcccgctgcagcaagttctatagggtcatctgtaaaacagctttacaagacctcaaaatctacaggttctgattataaaacacagatatcagcaggttatgatgcttacaaatccatcaatgatgcctatggtagggcacgaaatgatgcgactcgtgatgtgataaacaatgtaagtacccccgctctcacacgatccgttcctagagcacgaacagaagctcgaggaccagaaaaaacagatcaagccctcgctagggtgatttctggcaatagcagaactcttggagatgtctatagtcaagtttcggcactacaatctgtaatgcagatcatccagtcgaatcctcaagcgaataatgaggagatcagacaaaagcttacatcggcagtgacaaagcctccacagtttggctatccttatgtgcaactttctaatgactctacacagaagttcatagctaaattagaaagtttgtttgctgaaggatctaggacagcagctgaaataaaagcactttcctttgaaacgaactccttgtttattcagcaggtgctggtcaatatcggctctctatattctggttatctccaataa

SEQ ID NO: 62 - protein, 651 aa, Chlamydia peneumoniae
MVNPIGPGPIDETERTPPADLSAQGLEASAANKSAEAQRIAGAEAKPKESKTDSVERWSILRSAVNALMSLADKLGIASSNSSSSTSRSADVDSTTATAPTPPPPTFDDYKTQAQTAYDTIFTSTSLADIQAALVSLQDAVTNIKDTAATDEETAIAAEWETKNADAVKVGAQITELAKYASDNQAILDSLGKLTSFDLLQAALLQSVANNNKAAELLKEMQDNPVVPGKTPAIAQSLVDQTDATATQIEKDGNAIRDAYFAGQNASGAVENAKSNNSISNIDSAKAAIATAKTQIAEAQKKFPDSPILQEAEQMVIQAEKDLKNIKPADGSDVPNPGTTVGGSKQQGSSIGSIRVSMLLDDAENETASILMSGFRQMIHMFNTENPDSQAAQQELAAQARAAKAAGDDSAAAALADAQKALEAALGKAGQQQGILNALGQIASAAVVSAGVPPAAASSIGSSVKQLYKTSKSTGSDYKTQISAGYDAYKSINDAYGRARNDATRDVINNVSTPALTRSVPRARTEARGPEKTDQALARVISGNSRTLGDVYSQVSALQSVMQIIQSNPQANNEEIRQKLTSAVTKPPQFGYPYVQLSNDSTQKFIAKLESLFAEGSRTAAEIKALSFETNSLFIQQVLVNIGSLYSGYLQ

SEQ ID NO: 63 - DNA, 261 bp, Chlamydia trachomatis
atgagtcaaaataagaactctgctttcatgcagcctgtgaacgtatccgctgatttagctgccatcgttggtgcaggacctatgcctcgcacagagatcattaagaaaatgtgggattacattaagaagaatggccttcaagatcctacaaacaaacgtaatatcaatcccgatgataaattggctaaagtttttggaactgaaaaacctatcgatatgttccaaatgacaaaaatggtttctcaacacatcattaaataa

SEQ ID NO: 64 - protein, 86 aa, Chlamydia trachomatis
MSQNKNSAFMQPVNVSADLAAIVGAGPMPRTEIIKKMWDYIKKNGLQDPTNKRNINPDDKLAKVFGTEKPIDMFQMTKMVSQHIIK

SEQ ID NO: 65 - DNA, 261 bp, Chlamydia muridarum
atgagtcaaaataagaactctgctttcatgcagcctgtgaacgtatcttctgatttagctgccattgttggtacagggcctatgcctcgcacagaaatcattaagaaaatttgggattatattaagcagaataaacttcaagatcctactaacaaacgcaacatcaatcctgatgataaattagccaaggtttttggttccaaagaccctgtagatatgttccaaatgacaaaaatagtctctaaacacattgttaaataa

SEQ ID NO: 66 - protein, 86 aa, Chlamydia muridarum
MSQNKNSAFMQPVNVSSDLAAIVGTGPMPRTEIIKKIWDYIKQNKLQDPTNKRNINPDDKLAKVFGSKDPVDMFQMTKIVSKHIVK

SEQ ID NO: 67 - DNA, 264 bp, Chlamydia psitacci
atgagtcaaaaaaacaaaaactctgcttttatgaaccccgtcaatattacccccgatttagcagctatcgttggcgagggaccaatgccccgcactgaaattgtcaaaaaagtatgggagcacattaaaaaaaataaccttcaagaccctaagaataaaagaaatatccttcccgatgacgccctagctaaagtctttggttctaaaaatccaatcgatatgtttcaaatgacgaaagccctttccgctcatatcgtaaaataa

SEQ ID NO: 68 - protein, 87 aa, Chlamydia psitacci
MSQKNKNSAFMNPVNITPDLAAIVGEGPMPRTEIVKKVWEHIKKNNLQDPKNKRNILPDDALAKVFGSKNPIDMFQMTKALSAHIVK

SEQ ID NO: 69 - DNA, 264 bp, Chlamydia pneumoniae
atgagtcaaaaaaataaaaactctgcttttatgcatcccgtgaatatttccacagatttagcagttatagttggcaagggacctatgcccagaaccgaaattgtaaagaaagtttgggaatacattaaaaaacacaactgtcaggatcaaaaaaataaacgtaatatccttcccgatgcgaatcttgccaaagtctttggctctagtgatcctatcgacatgttccaaatgaccaaagccctttccaaacatattgtaaaataa

SEQ ID NO: 70 - protein, 87 aa, Chlamydia pneumoniae
MSQKNKNSAFMHPVNISTDLAVIVGKGPMPRTEIVKKVWEYIKKHNCQDQKNKRNILPDANLAKVFGSSDPIDMFQMTKALSKHIVK

SEQ ID NO: 71 - DNA, 1266 bp, Chlamydia trachomatis
atgactgcatcaggaggagctggagggctaggcagcacccaaacagtagacgttgcgcgagcacaagctgctgcagctactcaagatgcacaagaggttatcggctctcaggaagcttctgaggcaagtatgctcaaaggatgtgaggatctcataaatcctgcagctgcaacccgaatcaaaaaaaaaggagagaagtttgaatcattagaagctcgtcgcaaaccaacagcggataaagcagaaaagaaatccgagagcacagaggaaaaaggcgatactcctcttgaagatcgtttcacagaagatctttccgaagtctccggagaagattttcgaggattgaaaaattcgttcgatgatgattcttctcctgacgaaattctcgatgcgctcacaagtaaattttctgatcccacaataaaggatctagctcttgattatctaattcaaacagctccctctgatgggaaacttaagtccactctcattcaggcaaagcatcaactgatgagccagaatcctcaggcgattgttggaggacgcaatgttctgttagcttcagaaacctttgcttccagagcaaatacatctccttcatcgcttcgctccttatatttccaagtaacctcatccccctctaattgcgctaatttacatcaaatgcttgcttcttacttgccatcagagaaaaccgctgttatggagtttctagtaaatggcatggtagcagatttaaaatcggagggcccttccattcctcctgcaaaattgcaagtatatatgacggaactaagcaatctccaagccttacactctgtaaatagcttttttgatagaaatattgggaacttggaaaatagcttaaagcatgaaggacatgcccctattccatccttaacgacaggaaatttaactaaaaccttcttacaattagtagaagataaattcccttcctcttccaaagctcaaaaggcattaaatgaactggtaggcccagatactggtcctcaaactgaagttttaaacttattcttccgcgctcttaatggctgttcgcctagaatattctctggagctgaaaaaaaacagcagctggcatcggttatcacaaatacgctagatgcgataaatgcggataatgaggattatcctaaaccaggtgacttcccacgatcttccttctctagtacgcctcctcatgctccagtacctcaatctgagattccaacgtcacctacctcaacacagcctccatcaccctaa

SEQ ID NO: 72 - protein, 421 aa, Chlamydia trachomatis
MTASGGAGGLGSTQTVDVARAQAAAATQDAQEVIGSQEASEASMLKGCEDLINPAAATRIKKKGEKFESLEARRKPTADKAEKKSESTEEKGDTPLEDRFTEDLSEVSGEDFRGLKNSFDDDSSPDEILDALTSKFSDPTIKDLALDYLIQTAPSDGKLKSTLIQAKHQLMSQNPQAIVGGRNVLLASETFASRANTSPSSLRSLYFQVTSSPSNCANLHQMLASYLPSEKTAVMEFLVNGMVADLKSEGPSIPPAKLQVYMTELSNLQALHSVNSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 73 - DNA, 1257 bp, Chlamydia muridarum
atgactgcatccggaggagctggagggttaggcggaacccaaacagtaaacgtagcacaagcgcaagctgcagcagctactcaggatgcacaagaaatcataggctctcaggaagcttctgaagccagtttgattaaaggaagtgaggatcttgctaatcctgctgcagcgactagaatcaaaaagaaagaagacaaatttcagtcattagaagctcgtcgaaaaacaactagtaaatccgaaaaaaaatcagaaagtacagaagagaaatcagactcttctcttgaagagcgcttcacagaaaatctttcggatgtttctggagaagattttcgagggttaaaggattctctgagtgaagattcctctcctgaagagattcttgagaagctgtcaggcaaattttcggaccccacaattaaagatcttgctctagactttctgattcaatcgagtcctcctgatgggaaattaagagcctctcttattcaggcaaaacagacgctttttcaacaaaatcctcaagcagtcaaaggagggcgcaacgttcttttagcatcagaagcctttgcttctaaagcaaacacttcccctgcatcattacgcgcattgtatacccaagtaacctcatctccggctaattgtgcttctctaagtcagatgctatcctcttattctcctacagaaaaagcagctgttatagattttttaacaaatggtatggtgtctgatctcaaatcaggagggccttccatccctgctccacaattgcaagtgtatatgacggagctcagcaatctacaagccctcaactctgtagacagtttttttgacaaaaatacaaaaggactagaagacaatttaaaagccgaaggacatacccttccaccatccctaactcccagtaatcttgctcaaacttttttaaagttagtggaagataagttcccgtcctcccaaaaagctcaaaaattgttggatggccttgttggttctgacgttactcctcaaactgaagttttaaatctcttttaccgagcgctcaatggttgttccccacgaatattcggcaatgctgagaaaaaacagcagctagcaacagtaattactaacacattagataccgtgaatgccgataacgaagattatcctaaacctagcgatttccccaaaccttccttccatggcactcctcctcatgctccagtgtctctatctgatattccatcagcaacaacaaactctgcagaccaataa

SEQ ID NO: 74 - protein, 418 aa, Chlamydia muridarum
MTASGGAGGLGGTQTVNVAQAQAAAATQDAQEIIGSQEASEASLIKGSEDLANPAAATRIKKKEDKFQSLEARRKTTSKSEKKSESTEEKSDSSLEERFTENLSDVSGEDFRGLKDSLSEDSSPEEILEKLSGKFSDPTIKDLALDFLIQSSPPDGKLRASLIQAKQTLFQQNPQAVKGGRNVLLASEAFASKANTSPASLRALYTQVTSSPANCASLSQMLSSYSPTEKAAVIDFLTNGMVSDLKSGGPSIPAPQLQVYMTELSNLQALNSVDSFFDKNTKGLEDNLKAEGHTLPPSLTPSNLAQTFLKLVEDKFPSSQKAQKLLDGLVGSDVTPQTEVLNLFYRALNGCSPRIFGNAEKKQQLATVITNTLDTVNADNEDYPKPSDFPKPSFHGTPPHAPVSLSDIPSATTNSADQ

SEQ ID NO: 75 - DNA, 1194 bp, Chlamydia psitacci
atggctgcatctggaggagctggtggcttaggcggttcacaagctgttgacgttgcgcaagtgcaagctgcagctgcgaaagctgatgcccaagaagttatcgctagccaagagcaatccgacatcagtatgattaaggattctcaggatttatcaaatcctcaggctgcgacacgtacaaagaaaaaagaagaaaaattccaaactctagaatctagaaggaaaggcgcgactcaagcagagaaaaagtctgaaagcacgggagataaatccgacgcggatcttgcggataagtatacagaaaataatgctgaaatctcaggtcaagatttacgcagtatccgagattctttgcatgatggttcttccgaagaagatgttttagatcttgtaaaatctaagttctctgatcctgcgcttcaaagtgttgccctagattatttagtccagacaacaccagcttctaaaggagctttaaaagacaccttaatcagggcacaacaaaaccacatgcaacaaaatcgacaagctgttgttggtggtaaaaatattctatttgcctctcaagagtatgcatctttattaaatacctctgctccaggattacgtgctctttatcttgaggtaacgtctgatttccattcttgtgagcaattactaacatctctccagtcacgttatagttacgaagaaatgggcactgtttcctctttcatacttaaggggatggctgctgatttaaaatctgaaggatcttcaattccagctccgaaactacaggtgatgatgacagaaactcgtaaccttcaagctgtgcttactggttatcatttctttgagacaaagctaccaacacttaccgcatctttaaaagccgatggggtaacagttccggatcttaaatttgataaagtagccgatactttctttaagttaatcaatgataaattccctacggcttcaaaaatggagcgcggtgtccgtgaccttattggcgacgatacagaagctgttacagggatgctcaacctcttctttgttgctttaagggggacatccccaagattatttgcttcagcagaaaagcgtcagcaattaggcacaatgatggctaatgctttagatgctgtgaatattaacaacgaagattacccaaaatctacagacttccccaaaccttatccctggtcttaa

SEQ ID NO: 76 - protein, 397 aa, Chlamydia psitacci
MAASGGAGGLGGSQAVDVAQVQAAAAKADAQEVIASQEQSDISMIKDSQDLSNPQAATRTKKKEEKFQTLESRRKGATQAEKKSESTGDKSDADLADKYTENNAEISGQDLRSIRDSLHDGSSEEDVLDLVKSKFSDPALQSVALDYLVQTTPASKGALKDTLIRAQQNHMQQNRQAVVGGKNILFASQEYASLLNTSAPGLRALYLEVTSDFHSCEQLLTSLQSRYSYEEMGTVSSFILKGMAADLKSEGSSIPAPKLQVMMTETRNLQAVLTGYHFFETKLPTLTASLKADGVTVPDLKFDKVADTFFKLINDKFPTASKMERGVRDLIGDDTEAVTGMLNLFFVALRGTSPRLFASAEKRQQLGTMMANALDAVNINNEDYPKSTDFPKPYPWS

SEQ ID NO: 77 - DNA, 1200 bp, Chlamydia pneumoniae
atggcagcatcaggaggcacaggtggtttaggaggcactcagggtgtcaaccttgcagctgtagaagctgcagctgcaaaagcagatgcagcagaagttgtagccagccaagaaggttctgagatgaacatgattcaacaatctcaggacctgacaaatcccgcagcagcaacacgcacgaaaaaaaaggaagagaagtttcaaactctagaatctcggaaaaaaggagaagctggaaaggctgagaaaaaatctgaatctacagaagagaagcctgacacagatcttgctgataagtatgcttctgggaattctgaaatctctggtcaagaacttcgcggcctgcgtgatgcaataggagacgatgcttctccagaagacattcttgctcttgtacaagagaaaattaaagacccagctctgcaatccacagctttggactacctggttcaaacgactccaccctcccaaggtaaattaaaagaagcgcttatccaagcaaggaatactcatacggagcaattcggacgaactgctattggtgcgaaaaacatcttatttgcctctcaagaatatgcagaccaactgaatgtttctccttcagggcttcgctctttgtacttagaagtgactggagacacacatacctgtgatcagctactttctatgcttcaagaccgctatacctaccaagatatggctattgtcagctcctttctaatgaaaggaatggcaacagaattaaaaaggcagggtccctacgtacccagtgcgcaactacaagttctcatgacagaaactcgtaacctgcaagcagttcttacctcgtacgattactttgaaagtcgcgttcctattttactcgatagcttaaaagctgagggaatccaaactccttctgatctaaactttgtgaaggtagctgagtcctaccataaaatcattaacgataagttcccaacagcatctaaagtagaacgagaagtccgcaatctcataggagacgatgttgattctgtgaccggtgtcttgaacttattcttttctgctttacgtcaaacgtcgtcacgccttttctcttcagcagacaaacgtcagcaattaggagctatgattgctaatgctttagatgctgtaaatataaacaatgaagattatcccaaagcatcagacttccctaaaccctatccttggtcatga

SEQ ID NO: 78 - protein, 399 aa, Chlamydia pneumoniae
MAASGGTGGLGGTQGVNLAAVEAAAAKADAAEVVASQEGSEMNMIQQSQDLTNPAAATRTKKKEEKFQTLESRKKGEAGKAEKKSESTEEKPDTDLADKYASGNSEISGQELRGLRDAIGDDASPEDILALVQEKIKDPALQSTALDYLVQTTPPSQGKLKEALIQARNTHTEQFGRTAIGAKNILFASQEYADQLNVSPSGLRSLYLEVTGDTHTCDQLLSMLQDRYTYQDMAIVSSFLMKGMATELKRQGPYVPSAQLQVLMTETRNLQAVLTSYDYFESRVPILLDSLKAEGIQTPSDLNFVKVAESYHKIINDKFPTASKVEREVRNLIGDDVDSVTGVLNLFFSALRQTSSRLFSSADKRQQLGAMIANALDAVNINNEDYPKASDFPKPYPWS

SEQ ID NO: 79 - DNA, 1266 bp, Chlamydia trachomatis
atgactgcatcaggaggagctggagggctaggcagcacccaaacagtagacgttgcgcgagcacaagctgctgcagctactcaagatgcacaagaggttatcggctctcaggaagcttctgaggcaagtatgctcaaaggatgtgaggatctcataaatcctgcagctgcaacccgaatcaaaaaaaaagaagagaagtttgaatcattagaagctcgtcgcaaaccaacagcggataaagcagaaaagaaatccgagagcacagaggaaaaaggcgatactcctcttgaagatcgtttcacagaagatctttccgaagtctccggagaagattttcgaggattgaaaaattcgttcgatgatgattcttctcctgaagaaattctcgatgcgctcacaagtaaattttctgatcccacaataaaggatctagctcttgattatctaattcaaacagctccctctgataggaaacttaagtccgctctcattcaggcaaagcatcaactgatgagccagaatcctcaggcgattgttggaggacgcaatgttctgttagcttcagaaacctttgcttccagagcaaatacatctccttcatcgcttcgctccttatatttccaagtaacctcatccccctctaattgtgataatttacgtcaaatgcttgcttcttactcgccatcagagaaaaccgctgttatggagtttctagtaaatggcatggtagcagatttaaaatcggagggcccttccattcctcctgcaaaattgcaagtatatatgacggaactaagcaatctccaagccttacactctgtagatagcttttttgatagaaatattgggaacttggaaaatagcttaaagcatgaaggacatgcccctattccatccttaacgacaggaaatttaactaaaaccttcttacaattagtagaagataaattcccttcctcttccaaagctcaaaaggcattaaatgaactggtaggcccagatactggtcctcaaactgaagttttaaacttattcttccgcgctcttaatggctgttcgcctagaatattctctggagctgaaaaaaaacagcagctggcatcggttatcacaaatacgctagatgcgataaatgcggataatgaggattatcctaaaccaggtgacttcccacgatcttccttctctagtacgcctcctcatgctccagtacctcaatctgagattccaacgtcacctacctcaacacagcctccatcaccctaa

SEQ ID NO: 80 - protein, 421 aa, Chlamydia trachomatis
MTASGGAGGLGSTQTVDVARAQAAAATQDAQEVIGSQEASEASMLKGCEDLINPAAATRIKKKEEKFESLEARRKPTADKAEKKSESTEEKGDTPLEDRFTEDLSEVSGEDFRGLKNSFDDDSSPEEILDALTSKFSDPTIKDLALDYLIQTAPSDRKLKSALIQAKHQLMSQNPQAIVGGRNVLLASETFASRANTSPSSLRSLYFQVTSSPSNCDNLRQMLASYSPSEKTAVMEFLVNGMVADLKSEGPSIPPAKLQVYMTELSNLQALHSVDSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 81 - DNA, 1266 bp, Chlamydia trachomatis
atgactgcatcaggaggagctggagggctaggcagcacccaaacagtagacgttgcgcgagcacaagctgctgcagctactcaagatgcacaagaggttatcggctctcaggaagcttctgaggcaagtatgctcaaaggatgtgaggatctcataaatcctgcagctgcaacccgaatcaaaaaaaaagaagagaagtttgaatcattagaagctcgtcgcaaaccaacagcggataaagcagaaaagaaatccgagagcacagaggaaaaaggcgatactcctcttgaagatcgtttcacagaagatctttccgaagtctccggagaagattttcgaggattgaaaaattcgttcgatgatgattcttctcctgaagaaattctcgatgcgctcacaagtaaattttctgatcccacaataaaggatctagctcttgattatctaattcaaacagctccctctgataggaaacttaagtccgctctcattcaggcaaagcatcaactgatgagccagaatcctcaggcgattgttggaggacgcaatgttctgttagcttcagaaacctttgcttccagagcaaatacatctccttcatcgcttcgctccttatatctccaagtaacctcatccccctctaattgtgataatttacgtcaaatgcttgcttcttactcgccatcagagaaaaccgctgttatggagtttctagtaaatggcatggtagcagatttaaaatcggagggcccttccattcctcctgcaaaattgcaagtatatatgacggaactaagcaatctccaagccttacactctgtagatagcttttttgatagaaatattgggaacttggaaaatagcttaaagcatgaaggacatgcccctattccatccttaacgacaggaaatttaactaaaaccttcttacaattagtagaagataaattcccttcctcttccaaagctcaaaaggcattaaatgaactggtaggcccagatactggtcctcaaactgaagttttaaacttattcttccgcgctcttaatggctgttcgcctagaatattctctggagctgaaaaaaaacagcagctggcatcggttatcacaaatacgctagatgcgataaatgcggataatgaggattatcctaaaccaggtgacttcccacgatcttccttctctagtacgcctcctcatgctccagtacctcaatctgagattccaacgtcacctacctcaacacagcctccatcaccctaa

SEQ ID NO: 82 - protein, 421 aa, Chlamydia trachomatis
MTASGGAGGLGSTQTVDVARAQAAAATQDAQEVIGSQEASEASMLKGCEDLINPAAATRIKKKEEKFESLEARRKPTADKAEKKSESTEEKGDTPLEDRFTEDLSEVSGEDFRGLKNSFDDDSSPEEILDALTSKFSDPTIKDLALDYLIQTAPSDRKLKSALIQAKHQLMSQNPQAIVGGRNVLLASETFASRANTSPSSLRSLYLQVTSSPSNCDNLRQMLASYSPSEKTAVMEFLVNGMVADLKSEGPSIPPAKLQVYMTELSNLQALHSVDSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 83 - DNA, 1266 bp, Chlamydia trachomatis; identical to SEQ ID NO: 71

SEQ ID NO: 84 - protein, 421 aa, Chlamydia trachomatis; identical to SEQ ID NO: 72

SEQ ID NO: 85 - DNA, 1266 bp, Chlamydia trachomatis
atgactgcatcaggaggagctggagggctaggcagcacccaaacagtagacgttgcgcgagcacaagctgctgcagctactcaagatgcacaagaggttatcggctctcaggaagcttctgaggcaagtatgctcaaaggatgtgaggatctcataaatcctgcagctgcaacccgaatcaaaaaaaaagaagagaagtttgaatcattagaagctcgtcgcaaaccaacagcggataaagcagaaaagaaatccgagagcacagaggaaaaaggcgatactcctcttgaagatcgtttcacagaagatctttccgaagtctccggagaagattttcgaggattgaaaaattcgttcgatgatgattcttctcctgacgaaattctcgatgcgctcacaagtaaattttctgatcccacaataaaggatctagctcttgattatctaattcaaacagctccctctgatgggaaacttaagtccgctctcattcaggcaaagcatcaactgatgagccagaatcctcaggcgattgttggaggacgcaatgttctgttagcttcagaaacctttgcttccagagcaaatacatctccttcatcgcttcgctccttatatttccaagtaacctcatccccctctaattgcgctaatttacatcaaatgcttgcttcttacttgccatcagagaaaaccgctgttatggagtttctagtaaatggcatggtagcagatttaaaatcggagggcccttccattcctcctgcaaaattgcaagtatatatgacggaactaagcaatctccaagccttacactctgtaaatagcttttttgatagaaatattgggaacttggaaaatagcttaaagcatgaaggacatgcccctattccatccttaacgacaggaaatttaactaaaaccttcttacaattagtagaagataaattcccttcctcttccaaagctcaaaaggcattaaatgaactggtaggcccagatactggtcctcaaactgaagttttaaacttattcttccgcgctcttaatggctgttcgcctagaatattctctggagctgaaaaaaaacagcagctggcatcggttatcacaaatacgctagatgcgataaatgcggataatgaggattatcctaaaccaggtgacttcccacgatcttccttctctagtacgcctcctcatgctccagtacctcaatctgagattccaacgtcacctacctcaacacagcctccatcaccctaa

SEQ ID NO: 86 - protein, 421 aa, Chlamydia trachomatis
MTASGGAGGLGSTQTVDVARAQAAAATQDAQEVIGSQEASEASMLKGCEDLINPAAATRIKKKEEKFESLEARRKPTADKAEKKSESTEEKGDTPLEDRFTEDLSEVSGEDFRGLKNSFDDDSSPDEILDALTSKFSDPTIKDLALDYLIQTAPSDGKLKSALIQAKHQLMSQNPQAIVGGRNVLLASETFASRANTSPSSLRSLYFQVTSSPSNCANLHQMLASYLPSEKTAVMEFLVNGMVADLKSEGPSIPPAKLQVYMTELSNLQALHSVNSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 87 - DNA, 1266 bp, Chlamydia trachomatis; identical to SEQ ID NO: 71

SEQ ID NO: 88 - protein, 421 aa, Chlamydia trachomatis; identical to SEQ ID NO: 72

SEQ ID NO: 89 - DNA, 1266 bp, Chlamydia trachomatis
atgactgcatcaggaggagctggagggctaggcagcacccaaacagtagacgttgcgcgagcacaagctgctgcagctactcaagatgcacaagaggttatcggctctcaggaagcttctgaggcaagtatgctcaaagaatgtgcggatctcataaatcctgcagctgcaacccgaatcaaaaaaaaaaaagagaagtttgaatcattagaagctcgtcgcaaaccaacagcggataaagcagaaaagaaatccgagagcacagaggaaaaaggcgatactcctcttgaagatcgtttcacagaagatctttccgaagtctctggagaagattttcgaggattgaaaaattcgttcgatgatgattcttcttctgacgaaattctcgatgcgctcacaagtaaattttctgatcccacaataaaggatctagctcttgattatctaattcaaatagctccctctgatgggaaacttaagtccactctcattcaggcaaagcatcaactgatgagccagaatcctcaggcgattgttggaggacgcaatgttctgttagcttcagaaacctttgcttccagagcaaatacatctccttcatcgcttcgctccttatatctccaagtaacctcatccccctctaattgcgctaatttacatcaaatgcttgcttcttactcgccatcagagaaaaccgctgttatggagtttctagtgaatggcatggtagcagatttaaaatcggagggcccttccattcctcctgcaaaattgcaagtatatatgacggaactaagcaatctccaagccttacactctgtagatagcttttttgatagaaatattgggaacttggaaaatagcttaaagcatgaaggacatgcccctattccatccttaacgacaggaaatttaactaaaaccttcttacaattagtagaagataaattcccttcctcttccaaagctcaaaaggcattaaatgaactggtaggcccagatactggtcctcaaactgaagttttaaacttattcttccgcgctcttaatggctgttcgcctagaatattctctggagctgaaaaaaaacagcagctggcatcggttatcacaaatacgctagatgcgataaatgcggataatgaggattatcctaaaccaggtgacttcccacgatcttccttctctagtacgcctcctcatgctccagtacctcaatctgagattccaacgtcacctacctcaacacagcctccatcaccctaa

SEQ ID NO: 90 - protein, 421 aa, Chlamydia trachomatis
MTASGGAGGLGSTQTVDVARAQAAAATQDAQEVIGSQEASEASMLKECADLINPAAATRIKKKKEKFESLEARRKPTADKAEKKSESTEEKGDTPLEDRFTEDLSEVSGEDFRGLKNSFDDDSSSDEILDALTSKFSDPTIKDLALDYLIQIAPSDGKLKSTLIQAKHQLMSQNPQAIVGGRNVLLASETFASRANTSPSSLRSLYLQVTSSPSNCANLHQMLASYSPSEKTAVMEFLVNGMVADLKSEGPSIPPAKLQVYMTELSNLQALHSVDSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 91 - DNA, 1266 bp, Chlamydia trachomatis; identical to SEQ ID NO: 71

SEQ ID NO: 92 - protein, 421 aa, Chlamydia trachomatis; identical to SEQ ID NO: 72

SEQ ID NO: 93 - DNA, 1266 bp, Chlamydia trachomatis
atgactgcatcaggaggagctggagggctaggcagcacccaaacagtagacgttgcgcgagcacaagctgctgcagctactcaagatgcacaagaggttatcggctctcaggaagcttctgaggcaagtatgctcaaagaatgtgaggatctcataaatcctgcagctgcaacccgaatcaaaaaaaaagaagagaagtttgaatcattagaagctcgtcgcaaaccaacagcggataaagcagaaaagaaatccgagagcacagaggaaaaaggcgatactcctcttgaagatcgtttcacagaagatctttccgaagtctctggagaagattttcgaggattgaaaaattcgttcgatgatgattcttcttctgacgaaattctcgatgcgctcacaagtaaattttctgatcccacaataaaggatctagctcttgattatctaattcaaatagctccctctgatgggaaacttaagtccgctctcattcaggcaaagcatcaactgatgagccagaatcctcaggcgattgttggaggacgcaatgttctgttagcttcagaaacctttgcttccagagcaaatacatctccttcatcgcttcgctccttatatttccaagtaacctcatccccctctaattgcgctaatttacatcaaatgcttgcttcttactcgccatcagagaaaaccgctgttatggagtttctagtgaatggcatggtagcagatttaaaatcggagggcccttccattcctcctgcaaaattgcaagtatatatgacggaactaagcaatctccaagccttacactctgtagatagcttttttgatagaaatattgggaacttggaaaatagcttaaagcatgaaggacatgcccctattccatccttaacgacaggaaatttaactaaaaccttcttacaattagtagaagataaattcccttcctcttccaaagctcaaaaggcattaaatgaactggtaggcccggatactggtcctcaaactgaagttttaaacttattcttccgcgctcttaatggctgttcgcctagaatattctctggagctgaaaaaaaacagcagctggcatcggttatcacaaatacgctagatgcgataaatgcggataatgaggattatcctaaaccaggtgacttcccacgatcttccttctctagtacgcctcctcatgctccagtacctcaatctgagattccaacgtcacctacctcaacacagcctccatcaccctaa

SEQ ID NO: 94 - protein, 421 aa, Chlamydia trachomatis
MTASGGAGGLGSTQTVDVARAQAAAATQDAQEVIGSQEASEASMLKECEDLINPAAATRIKKKEEKFESLEARRKPTADKAEKKSESTEEKGDTPLEDRFTEDLSEVSGEDFRGLKNSFDDDSSSDEILDALTSKFSDPTIKDLALDYLIQIAPSDGKLKSALIQAKHQLMSQNPQAIVGGRNVLLASETFASRANTSPSSLRSLYFQVTSSPSNCANLHQMLASYSPSEKTAVMEFLVNGMVADLKSEGPSIPPAKLQVYMTELSNLQALHSVDSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 95 - DNA, 1749 bp, Chlamydia trachomatis
atggtacaaggagaaagcttggtttgcaagaatgctcttcaagatttgagttttttagagcatttattacaggttaaatatgctcctaaaacatggaaagagcaatacttaggatgggatcttgttcaaagctccgtttctgcacagcagaagcttcgtacacaagaaaatccatcaacaagtttttgccagcaggtccttgctgattttatcggaggattaaatgactttcacgctggagtaactttctttgcgatagaaagtgcttaccttccttataccgtacaaaaaagtagtgacggccgtttctactttgtagatatcatgactttttcttcagagatccgtgttggagatgagttgctagaggtggatggggcgcctgtccaagatgtactcgctactctatatggaagcaatcacaaagggactgcagctgaagagtcggctgctttaagaacactattttctcgcatggcctctttagggcacaaagtaccttctgggcgcactactttaaagattcgtcgtccttttggtactacgagagaagttcgtgtgaaatggcgttatgttcctgaaggtgtaggagatttggctaccatagctccttctatcagggctccacagttacagaaatcgatgagaagctttttccttaagaaagatgatgcgtttcatcggtctagttcgctattctactctccaatggttccgcatttttgggcagagcttcgcaatcattatgcaacgagtggtttgaaaagcgggtacaatattgggagtaccgatgggtttctccctgtcattgggcctgttatatgggagtcggagggtcttttccgcgcttatatttcttcggtgactgatggggatggtaagagccataaagtaggatttctaagaattcctacatatagttggcaggacatggaagattttgatccttcaggaccgcctccttgggaagaatttgctaagattattcaagtattttcttctaatacagaagctttgattatcgaccaaacgaacaacccaggtggtagtgtcctttatctttatgcactgctttccatgttgacagaccgtcctttagaacttcctaaacatagaatgattctgactcaggatgaagtggttgatgctttagattggttaaccctgttggaaaacgtagacacaaacgtggartctcgccttgctctgggagacaacatggaaggatatactgtggatctacaggttgccgagtatttaaaaagctttggacgtcaagtattgaattgttggagtaaaggggatattgagttatcaacgcctattcctctttttggttttgagaagattcatccacatcctcgagttcaatactctaaaccgatttgtgttttgatcaatgagcaagacttttcttgtgctgacttcttccctgtagttttgaaagacaatgatcgagctcttattgttggtactcgaacagctggagctggaggatttgtctttaatgtgcagttcccaaatagaactggaataaaaacttgttctttaacaggatcattagctgttagagagcatggtgccttcattgagaacatcggagtcgaaccgcatatcgatctgccttttacagcgaatgatattcgctataaaggctattccgagtatcttgataaggtcaaaaaattggtttgtcagctgatcaataacgacggtaccattattcttgcggaagatggtagtttttag

SEQ ID NO: 96 - protein, 582 aa, Chlamydia trachomatis
MVQGESLVCKNALQDLSFLEHLLQVKYAPKTWKEQYLGWDLVQSSVSAQQKLRTQENPSTSFCQQVLADFIGGLNDFHAGVTFFAIESAYLPYTVQKSSDGRFYFVDIMTFSSEIRVGDELLEVDGAPVQDVLATLYGSNHKGTAAEESAALRTLFSRMASLGHKVPSGRTTLKIRRPFGTTREVRVKWRYVPEGVGDLATIAPSIRAPQLQKSMRSFFLKKDDAFHRSSSLFYSPMVPHFWAELRNHYATSGLKSGYNIGSTDGFLPVIGPVIWESEGLFRAYISSVTDGDGKSHKVGFLRIPTYSWQDMEDFDPSGPPPWEEFAKIIQVFSSNTEALIIDQTNNPGGSVLYLYALLSMLTDRPLELPKHRMILTQDEVVDALDWLTLLENVDTNVESRLALGDNMEGYTVDLQVAEYLKSFGRQVLNCWSKGDIELSTPIPLFGFEKIHPHPRVQYSKPICVLINEQDFSCADFFPVVLKDNDRALIVGTRTAGAGGFVFNVQFPNRTGIKTCSLTGSLAVREHGAFIENIGVEPHIDLPFTANDIRYKGYSEYLDKVKKLVCQLINNDGTIILAEDGSF

SEQ ID NO: 97 - DNA, 1749 bp, Chlamydia trachomatis
atggtacaaggagaaagcttggtttgcaagaatgctcttcaagatttgagttttttagagcatttattacaggttaaatatgctcctaaaacatggaaagagcaatacttaggatgggatcttgttcaaagctccgtttctgcacagcagaagcttcgtacacaagaaaatccatcaacaagtttttgccagcaggtccttgctgattttatcggaggattaaatgactttcacgctggagtaactttctttgcgatagaaagtgcttaccttccttataccgtacaaaaaagtagtgacggccgtttctactttgtagatatcatgactttttcttcagagatccgtgttggagatgagttgctagaggtggatggggcgcctgtccaagatgtactcgctactctatatggaagcaatcacaaagggactgcagctgaagagtcggctgctttaagaacactattttctcgcatggcctctttagggcacaaagtaccttctgggcgcactactttaaagattcgtcgtccttttggtactacgagagaagttcgtgtgaaatggcgttatgttcctgaaggtgtaggagatttggctaccatagctccttctatcagggctccacagttacagaaatcgatgagaagctttttccctaagaaagatgatgcgtttcatcggtctagttcgctattctactctccaatggttccgcatttttgggcagagcttcgcaatcattatgcaacgagtggtttgaaaagcgggtacaatattgggagtaccgatgggtttctccctgtcattgggcctgttatatgggagtcggagggtcttttccgcgcttatatttcttcggtgactgatggggatggtaagagccataaagtaggatttctaagaattcctacatatagttggcaggacatggaagattttgatccttcaggaccgcctccttgggaagaatttgctaagattattcaagtattttcttctaatacagaagctttgattatcgaccaaacgaacaacccaggtggtagtgtcctttatctttatgcactgctttccatgttgacagaccgtcctttagaacttcctaaacatagaatgattctgactcaggatgaagtggttgatgctttagattggttaaccctgttggaaaacgtagacacaaacgtggaatctcgccttgctctgggagacaacatggaaggatatactgtggatctacaggttgccgagtatttaaaaagctttggacgtcaagtattgaattgttggagtaaaggggatatcgagttatcaacgcctattcctctttttggttttgagaagattcatccacatcctcgagttcaatactctaaaccgatttgtgttttgatcaatgagcaagacttttcttgtgctgacttcttccctgtagttttgaaagacaatgatcgagctcttattgttggtactcgaacagctggagctggaggatttgtctttaatgtgcagttcccaaatagaactggaataaaaacttgttctttaacaggatcattagctgttagagagcatggtgccttcattgagaacatcggagtcgaaccgcatatcgatctgccttttacagcgaatgatattcgctataaaggctattccgagtatcttgataaggtcaaaaaattggtttgtcagctgatcaataacgacggtaccattattcttgcggaagatggtagtttttag

SEQ ID NO: 98 - protein, 582 aa, Chlamydia trachomatis
MVQGESLVCKNALQDLSFLEHLLQVKYAPKTWKEQYLGWDLVQSSVSAQQKLRTQENPSTSFCQQVLADFIGGLNDFHAGVTFFAIESAYLPYTVQKSSDGRFYFVDIMTFSSEIRVGDELLEVDGAPVQDVLATLYGSNHKGTAAEESAALRTLFSRMASLGHKVPSGRTTLKIRRPFGTTREVRVKWRYVPEGVGDLATIAPSIRAPQLQKSMRSFFPKKDDAFHRSSSLFYSPMVPHFWAELRNHYATSGLKSGYNIGSTDGFLPVIGPVIWESEGLFRAYISSVTDGDGKSHKVGFLRIPTYSWQDMEDFDPSGPPPWEEFAKIIQVFSSNTEALIIDQTNNPGGSVLYLYALLSMLTDRPLELPKHRMILTQDEVVDALDWLTLLENVDTNVESRLALGDNMEGYTVDLQVAEYLKSFGRQVLNCWSKGDIELSTPIPLFGFEKIHPHPRVQYSKPICVLINEQDFSCADFFPVVLKDNDRALIVGTRTAGAGGFVFNVQFPNRTGIKTCSLTGSLAVREHGAFIENIGVEPHIDLPFTANDIRYKGYSEYLDKVKKLVCQLINNDGTIILAEDGSF

SEQ ID NO: 99 - DNA, 1749 bp, Chlamydia trachomatis
atggtacaaggagaaagcttggtttgcaagaatgctcttcaagatttgagttttttagagcatttattacaggttaaatatgctcctaaaacatggaaagagcaatacttaggatgggatcttgttcaaagctccgtttctgcacagcagaagcttcgtacacaagaaaatccatcaacaagtttttgccagcaggtccttgctgattttatcggaggattaaatgactttcacgctggagtaactttctttgcgatagaaagtgcttaccttccttataccgtacaaaaaagtagtgacggccgtttctactttgtagatatcatgactttttcttcagagatccgtgttggagatgagttgctagaggtggatggggcgcctgtccaagatgtactcgctactctatatggaagcaatcacaaagggactgcagctgaagagtcggctgctttaagaacactattttctcgcatggcctctttagggcacaaagtaccttctgggcgcactactttaaagattcgtcgtccttttggtactacgagagaagttcgtgtgaaatggcgttatgttcctgaaggtgtaggagatttggctaccatagctccttctatcagggctccacagttacagaaatcgatgagaagctttttccctaagaaagatgatgcgtttcatcggtctagttcgctattctactctccaatggttccgcatttttgggcagagcttcgcaatcattatgcaacgagtggtttgaaaagcgggtacaatattgggagtaccgatgggtttctccctgtcattgggcctgttatatgggagtcggagggtcttttccgcgcttatatttcttcggtgactgatggggatggtaagagccataaagtaggatttctaagaattcctacatatagttggcaggacatggaagattttgatccttcaggaccgcctccttgggaagaatttgctaagattattcaagtattttcttctaatacagaagctttgattatcgaccaaacgaacaacccaggtggtagtgtcctttatctttatgcactgctttccatgttgacagaccgtcctttagaacttcctaaacatagaatgattctgactcaggatgaagtggttgatgctttagattggttaaccctgttggaaaacgtagacacaaacgtggagtctcgccttgctctgggagacaacatggaaggatatactgtggatctacaggttgccgagtatttaaaaagctttggacgtcaagtattgaattgttggagtaaaggggatatcgagttatcaacgcctattcctctttttggttttgagaagattcatccacatcctcgagttcaatactctaaaccgatttgtgttttgatcaatgagcaagacttttcttgtgctgacttcttccctgtagttttgaaagacaatgatcgagctcttattgttggtactcgaacagctggagctggaggatttgtctttaatgtgcagttcccaaatagaactggaataaaaacttgttctttaacaggatcattagctgttagagagcatggtgccttcattgagaacatcggagtcgaaccgcatatcgatctgccttttacagcgaatgatattcgctataaaggctattccgagtatcttgataaggtcaaaaaattggtttgtcagctgatcaataacgacggtaccattattcttgcggaagatggtagtttttag

SEQ ID NO: 100 - protein, 582 aa, Chlamydia trachomatis; identical to SEQ ID NO: 98

SEQ ID NO: 101 - DNA, 1749 bp, Chlamydia trachomatis; identical to SEQ ID NO: 99

SEQ ID NO: 102 - protein, 582 aa, Chlamydia trachomatis; identical to SEQ ID NO: 98

SEQ ID NO: 103 - DNA, 1749 bp, Chlamydia trachomatis; identical to SEQ ID NO: 99

SEQ ID NO: 104 - protein, 582 aa, Chlamydia trachomatis; identical to SEQ ID NO: 98

SEQ ID NO: 105 - DNA, 1749 bp, Chlamydia trachomatis
atggtacgaggagaaagcttggtttgcaagaatgctcttcaagatttgagttttttagagcatttattacaggttaaatatgctcctaaaacatggaaagagcaatacttaggatgggatcttgttcaaagctccgtttctgcacagcagaagcttcgtacacaagaaaatccatcaacaagtttttgccagcaggtccttgctgattttatcggaggattaaatgactttcacgctggagtaactttctttgcgatagaaagtgcttaccttccttataccgtacaaaaaagtagtgacggccgtttctactttgtagatatcatgactttttcttcagagatccgtgttggagatgagttgctagaggtggatggggcgcctgtccaagatgtgctcgctactctatatggaagcaatcacaaagggactgcagctgaagagtcggctgctttaagaacactattttctcgcatggcctctttagggcacaaagtaccttctgggcgcactactttaaagattcgtcgtccttttggtactacgagagaagttcgtgtgaaatggcgttatgttcctgaaggtgtaggagatttggctaccatagctccttctatcagggctccacagttacagaaatcgatgagaagctttttccctaagaaagatgatgcgtttcatcggtctagttcgctattctactctccaatggttccgcatttttgggcagagcttcgcaatcattatgcaacgagtggtttgaaaagcgggtacaatattgggagtaccgatgggtttctccctgtcattgggcctgttatatgggagtcggagggtcttttccgcgcttatatttcttcggtgactgatggggatggtaagagccataaagtaggatttctaagaattcctacatatagttggcaggacatggaagattttgatccttcaggaccgcctccttgggaagaatttgctaagattattcaagtattttcttctaatacagaagctttgattatcgaccaaacgaacaacccaggtggtagtgtcctttatctttatgcactgctttccatgttgacagaccgtcctttagaacttcctaaacatagaatgattctgactcaggatgaagtggttgatgctttagattggttaaccctgttggaaaacgtagacacaaacgtggagtctcgccttgctctgggagacaacatggaaggatatactgtggatctacaggttgccgagtatttaaaaagctttggacgtcaagtattgaattgttggagtaaaggggatatcgagttatcaacacctattcctctttttggttttgagaagattcatccacatcctcgagttcaatactctaaaccgatttgtgttttgatcaatgagcaagacttttcttgtgctgacttcttccctgtagttttgaaagacaatgatcgagctcttattgttggtactcgaacagctggagctggaggatttgtctttaatgtgcagttcccaaatagaactggaataaaaacttgttctttaacaggatcattagctgttagagagcatggtgccttcattgagaacatcggagtcgaaccgcatatcgatctgccttttacagcgaatgatattcgctataaaggctattccgagtatcttgataaggtcaaaaaattggtttgtcagctgatcaataacgacggtaccattattcttgcggaagatggtagtttttag

SEQ ID NO: 106 - protein, 582 aa, Chlamydia trachomatis
MVRGESLVCKNALQDLSFLEHLLQVKYAPKTWKEQYLGWDLVQSSVSAQQKLRTQENPSTSFCQQVLADFIGGLNDFHAGVTFFAIESAYLPYTVQKSSDGRFYFVDIMTFSSEIRVGDELLEVDGAPVQDVLATLYGSNHKGTAAEESAALRTLFSRMASLGHKVPSGRTTLKIRRPFGTTREVRVKWRYVPEGVGDLATIAPSIRAPQLQKSMRSFFLKKDDAFHRSSSLFYSPMVPHFWAELRNHYATSGLKSGYNIGSTDGFLPVIGPVIWESEGLFRAYISSVTDGDGKSHKVGFLRIPTYSWQDMEDFDPSGPPPWEEFAKIIQVFSSNTEALIIDQTNNPGGSVLYLYALLSMLTDRPLELPKHRMILTQDEVVDALDWLTLLENVDTNVESRLALGDNMEGYTVDLQVAEYLKSFGRQVLNCWSKGDIELSTPIPLFGFEKIHPHPRVQYSKPICVLINEQDFSCADFFPVVLKDNDRALIVGTRTAGAGGFVFNVQFPNRTGIKTCSLTGSLAVREHGAFIENIGVEPHIDLPFTANDIRYKGYSEYLDKVKKLVCQLINNDGTIILAEDGSF

SEQ ID NO: 107 - DNA, 1749 bp, Chlamydia trachomatis; identical to SEQ ID NO: 99

SEQ ID NO: 108 - protein, 582 aa, Chlamydia trachomatis; identical to SEQ ID NO: 96

SEQ ID NO: 109 - DNA, 1749 bp, Chlamydia trachomatis; identical to SEQ ID NO: 105

SEQ ID NO: 110 - protein, 582 aa, Chlamydia trachomatis; identical to SEQ ID NO: 106

SEQ ID NO: 111 - DNA, 1770 bp, Chlamydia trachomatis
atgagcatcaggggagtaggaggcaacgggaatagtcgaatcccttctcataatggggatggatcgaatcgcagaagtcaaaatacgaagaataaagttgaagatcgagttcgttctctatattcatctcgtagtaacgaaaatagagaatctccttatgcagtagtagacgtcagctctatgatcgagagcaccccaacgagtggagagacgacaagagcttcgcgtggagtattcagtcgtttccaaagaggtttaggacgagtagctgacaaagtaagacgagctgttcagcgtgcgtggagttcagtctctataagaagatcgtctgcaacaagagccacagaatccagatcaagtagtcgtactgctcgtggtgcaagttctgggtataaggagtattctccttcagcagctagagggctgcgtcttatgttcacagatttctggagaactcgggttttacgccagacctctcctatggctggagtttttgggaatcttgatgtgaacgaggctcgtttgatggctgcgtacacaagtgagtgcgcggatcatttagaagcgaaggagttggctggccctgacggggtagcggccgcccgggaaattgctaaaagatgggagaaaagagttagagatctacaagataaaggtgctgcacgaaaattattaaatgatcctttaggccgacgaacacctaattatcagagcaaaaatccaggtgagtatactgtagggaattccatgttttacgatggtcctcaggtagcgaatctccagaacgtcgacactggtttttggctggacatgagcaatctctcagacgttgtattatccagagagattcaaacaggacttcgagcacgagctactttggaagaatccatgccgatgttagagaatttagaagagcgttttagacgtttgcaagaaacttgtgatgcggctcgtactgagatagaagaatcgggatggactcgagagtccgcatcaagaatggaaggcgatgaggcgcaaggaccttctagagcacaacaagcttttcagagctttgtaaatgaatgtaacagcatcgagttctcatttgggagctttggagagcatgtgcgagttctctgcgctagagtatcacgaggattagctgccgcaggagaggcgattcgccgttgcttctcttgttgtaaaggatcgacgcatcgctacgctcctcgcgatgacctatctcctgaaggtgcatcgttagcagagactttggctagattcgcagatgatatgggaatagagcgaggtgctgatggaacctacgatattcctttggtagatgattggagaagaggggttcctagtattgaaggagaaggatctgactcgatctatgaaatcatgatgcctatctatgaagttatgaatatggatctagaaacacgaagatcttttgcggtacagcaagggcactatcaggacccaagagcttcagattatgacctcccacgtgctagcgactatgatttgcctagaagcccatatcctactccacctttgcctcctagatatcagctacagaatatggatgtagaagcagggttccgtgaggcagtttatgcttcttttgtagcaggaatgtacaattatgtagtgacacagccgcaagagcgtattcccaatagtcagcaggtggaagggattctgcgtgatatgcttaccaacgggtcacagacatttagagacctgatgaagcgttggaatagagaagtcgatagggaataa

SEQ ID NO: 112 - protein, 589 aa, Chlamydia trachomatis
MSIRGVGGNGNSRIPSHNGDGSNRRSQNTKNKVEDRVRSLYSSRSNENRESPYAVVDVSSMIESTPTSGETTRASRGVFSRFQRGLGRVADKVRRAVQRAWSSVSIRRSSATRATESRSSSRTARGASSGYKEYSPSAARGLRLMFTDFWRTRVLRQTSPMAGVFGNLDVNEARLMAAYTSECADHLEAKELAGPDGVAAAREIAKRWEKRVRDLQDKGAARKLLNDPLGRRTPNYQSKNPGEYTVGNSMFYDGPQVANLQNVDTGFWLDMSNLSDVVLSREIQTGLRARATLEESMPMLENLEERFRRLQETCDAARTEIEESGWTRESASRMEGDEAQGPSRAQQAFQSFVNECNSIEFSFGSFGEHVRVLCARVSRGLAAAGEAIRRCFSCCKGSTHRYAPRDDLSPEGASLAETLARFADDMGIERGADGTYDIPLVDDWRRGVPSIEGEGSDSIYEIMMPIYEVMNMDLETRRSFAVQQGHYQDPRASDYDLPRASDYDLPRSPYPTPPLPPRYQLQNMDVEAGFREAVYASFVAGMYNYVVTQPQERIPNSQQVEGILRDMLTNGSQTFRDLMKRWNREVDRE

SEQ ID NO: 113 - DNA, 1770 bp, Chlamydia trachomatis
atgagcatcaggggagtaggaggcaacgggaatagtcgaatcccttctcataatggggatggatcgaatcgcagaagtcaaaatacgaagaataaagttgaagatcgagttcgttctctatattcatctcgtagtaacgaaaatagagaatctccttatgcagtagtagacgtcagctctatgatcgagagcaccccaacgagtggagagacgacaagagcttcgcgtggagtattcagtcgtttccaaagaggtttaggacgagtagctgacaaagtaagacgagctgttcagcgtgcgtggagttcagtctctataagaagatcgtctgcaacaagagccgcagaatccagatcaagtagtcgtactgctcgtggtgcaagttctgggtatagggagtattctccttcagcagctagagggctgcgtcttatgttcacagatttctggagaactcgggttttacgccagacctctcctatggctggagtttttgggaatcttgatgtgaacgaggctcgtttgatggctgcgtacacaagtgagtgcgcggatcatttagaagcgaaggagttggctggccctgacggggtagcggccgcccgggaaattgctaaaagatgggagaaaagagttagagatctacaagataaaggtgctgcacgaaaattattaaatgatcctttaggccgacgaacacctaattatcagagcaaaaatccaggtgagtatactgtagggaattccatgttttacgatggtcctcaggtagcgaatctccagaacgtcgacactggtttttggctggacatgagcaatctctcagacgttgtattatccagagagattcaaacaggacttcgagcacgagctactttggaagaatccatgccgatgttagagaatttagaagagcgttttagacgtttgcaagaaacttgtgatgcggctcgtactgagatagaagaatcgggatggactcgagagtccgcatcaagaatggaaggcgatgaggcgcaaggaccttctagagcacaacaagcttttcagagctttgtaaatgaatgtaacagcatcgagttctcatttgggagctttggagagcatgtgcgagttctctgcgctagagtatcacgaggattagctgccgcaggagaggcgattcgccgttgcttctcttgttgtaaaggatcgacgcatcgctacgctcctcgcgatgacctatctcctgaaggtgcatcgttagcagagactttggctagattcgcagatgatatgggaatagagcgaggtgctgatggaacctacgatattcctttggtagatgattggagaagaggggttcctagtattgaaggagaaggatctgactcgatctatgaaatcatgatgcctatctatgaagttatgaatatggatctagaaacacgaagatcttttgcggtacagcaagggcactatcaggacccaagagcttcagattatgacctcccacgtgctagcgactatgatttgcctagaagcccatatcctactccacctttgcctcctagatatcagctacagaatatggatgtagaagcagggttccgtgaggcagtttatgcttcttttgtagcaggaatgtacaattatgtagtgacacagccgcaagagcgtattcccaatagtcagcaggtggaagagattctgcgtgatatgcttaccaacgggtcacagacatttagagacctgatgaagcgttggaatagagaagtcgatagggaataa

SEQ ID NO: 114 - protein, 589 aa, Chlamydia trachomatis
MSIRGVGGNGNSRIPSHNGDGSNRRSQNTKNKVEDRVRSLYSSRSNENRESPYAVVDVSSMIESTPTSGETTRASRGVFSRFQRGLGRVADKVRRAVQRAWSSVSIRRSSATRAAESRSSSRTARGASSGYREYSPSAARGLRLMFTDFWRTRVLRQTSPMAGVFGNLDVNEARLMAAYTSECADHLEAKELAGPDGVAAAREIAKRWEKRVRDLQDKGAARKLLNDPLGRRTPNYQSKNPGEYTVGNSMFYDGPQVANLQNVDTGFWLDMSNLSDVVLSREIQTGLRARATLEESMPMLENLEERFRRLQETCDAARTEIEESGWTRESASRMEGDEAQGPSRAQQAFQSFVNECNSIEFSFGSFGEHVRVLCARVSRGLAAAGEAIRRCFSCCKGSTHRYAPRDDLSPEGASLAETLARFADDMGIERGADGTYDIPLVDDWRRGVPSIEGEGSDSIYEIMMPIYEVMNMDLETRRSFAVQQGHYQDPRASDYDLPRASDYDLPRSPYPTPPLPPRYQLQNMDVEAGFREAVYASFVAGMYNYVVTQPQERIPNSQQVEEILRDMLTNGSQTFRDLMKRWNREVDRE

SEQ ID NO: 115 - DNA, 1776 bp, Chlamydia trachomatis; identical to SEQ ID NO: 21

SEQ ID NO: 116 - protein, 591 aa, Chlamydia trachomatis; identical to SEQ ID NO: 22

SEQ ID NO: 117 - DNA, 1776 bp, Chlamydia trachomatis
atgagcatcaggggagtaggaggcaacgggaatagtcgaatcccttctcataatggggatggatcgaatcgcagaagtcaaaatacgaagggtaataataaagttgaagatcgagtttgttctctatattcatctcgtagtaacgaaaatagagaatctccttatgcagtagtagacgtcagctctatgatcgagagcaccccaacgagtggagagacgacaagagcttcgcgtggagtgttcagtcgtttccaaagaggtttagtacgagtagctgacaaagtaagacgagctgttcagtgtgcgtggagttcagtctctacaagaagatcgtctgcaacaagagccgcagaatccggatcaagtagtcgtactgctcgtggtgcaagttctgggtatagggagtattctccttcagcagctagagggctgcgtcttatgttcacagatttctggagaactcgggttttacgccagacctctcctatggctggagtttttgggaatcttgatgtgaacgaggctcgtttgatggctgcgtacacaagtgagtgcgcggatcatttagaagcgaacaagttggctggccctgacggggtagcggccgcccgggaaattgctaaaagatgggagcaaagagttagagatctacaagataaaggtgctgcacgaaaattattaaatgatcctttaggccgacgaacacctaattatcagagcaaaaatccaggtgagtatactgtagggaattccatgttttacgatggtcctcaggtagcgaatctccagaacgtcgacactggtttttggctggacatgagcaatctctcagacgttgtattatccagagagattcaaacaggacttcgagcacgagctactttggaagaatccatgccgatgttagagaatttagaagagcgttttagacgtttgcaagaaacttgtgatgcggctcgtactgagatagaagaatcgggatggactcgagagtccgcatcaagaatggaaggcgatgaggcgcaaggaccttctagagcacaacaagcttttcagagctttgtaaatgaatgtaacagcatcgagttctcatttgggagctttggagagcatgtgcgagttctctgcgctagagtatcacgaggattagctgccgcaggagaggcgattcgccgttgcttctcttgttgtaaaggatcgacgcatcgctacgctcctcgcgatgacctatctcctgaaggtgcatcgttagcagagactttggctagattcgcagatgatatgggaatagagcgaggtgctgatggaacctacgatattcctttggtagatgattggagaagaggggttcctagtattgaaggagaaggatctgactcgatctatgaaatcatgatgcctatctatgaagttatggatatggatctagaaacacgaagatcttttgcggtacagcaagggcactatcaggacccaagagcttcagattatgacctcccacgtgctagcgactatgatttgcctagaagcccatatcctactccacctttgcctcctagatatcagctacagaatatggatgtagaagcagggttccgtgaggcagtttatgcttcttttgtagcaggaatgtataattatgtagtgacacagccgcaagagcgtattcccaatagtcagcaggtggaagggattctgcgtgatatgcttaccaacgggtcacagacatttagagacctgatgaagcgttggaatagagaagtcgatagggaataa

SEQ ID NO: 118 - protein, 591 aa, Chlamydia trachomatis
MSIRGVGGNGNSRIPSHNGDGSNRRSQNTKGNNKVEDRVCSLYSSRSNENRESPYAVVDVSSMIESTPTSGETTRASRGVFSRFQRGLVRVADKVRRAVQCAWSSVSTRRSSATRAAESGSSSRTARGASSGYREYSPSAARGLRLMFTDFWRTRVLRQTSPMAGVFGNLDVNEARLMAAYTSECADHLEANKLAGPDGVAAAREIAKRWEQRVRDLQDKGAARKLLNDPLGRRTPNYQSKNPGEYTVGNSMFYDGPQVANLQNVDTGFWLDMSNLSDVVLSREIQTGLRARATLEESMPMLENLEERFRRLQETCDAARTEIEESGWTRESASRMEGDEAQGPSRAQQAFQSFVNECNSIEFSFGSFGEHVRVLCARVSRGLAAAGEAIRRCFSCCKGSTHRYAPRDDLSPEGASLAETLARFADDMGIERGADGTYDIPLVDDWRRGVPSIEGEGSDSIYEIMMPIYEVMDMDLETRRSFAVQQGHYQDPRASDYDLPRASDYDLPRSPYPTPPLPPRYQLQNMDVEAGFREAVYASFVAGMYNYVVTQPQERIPNSQQVEGILRDMLTNGSQTFRDLMKRWNREVDRE

SEQ ID NO: 119 - DNA, 1776 bp, Chlamydia trachomatis; identical to SEQ ID NO: 21

SEQ ID NO: 120 - protein, 591 aa, Chlamydia trachomatis; identical to SEQ ID NO: 22

SEQ ID NO: 121 - DNA, 1773 bp, Chlamydia trachomatis
atgagcatcaggggagtaggaggcaacgggaatagtcgaatcccttctcataatggggatggatcgaatcgcagaagtcaaaatacgaagggtaataataaagttgaagatcgagttcattctctatattcatctcttagtaacgaaaatagagaatctccttatccagtagtagacgtcagctctatgatcgagagcaccccaacgagtggagagacgccaagagcttcgcgtggagtgttcagtcgtttccaaagaggtttaggacgagtagctgacaaagtaagacgagctgttcagtgtgcgtggggttcagtctctacaagaagatcgtctgcaacaagagccgtagaatccggatcaagtagtcgtactgctcgtggtgcaagttctgggagggagtattctccttcagcagctagagggctgcgtcttatgttcacagatttctggagaactcgggttttacgccagacctctcctatggatgtagtttttgggaatcttgatgtgaacgaggctcgtttgatggctgcttacacaagtgagtgcgcggattatttagaagcgcacgatttggctggccctgacggggtagcggccgcccgggaaattgctcaaagatgggagaaaagagttagagatctacaagataaaggtgctgcacaaaaattattaaatgatcctttaggccgacgaacacctaattatcagagcaaaaatccaggtgagtatactgtagggaattccatgttttacgatggtcctcaggtagcgaatctccagaacgtcgacactggtttttggctggacatgagcaatttctcagacgttgtattatccagagagattcaaacagggcttcgagcacgagctactttggaagaatccatgccgatgttagagaatttagaagagcgttttagacgtttgcaagaaacttgtgatgcggctcgtactgagatagaagaatcgggatggactcgagagtccgcatcaagaatgggaggcgatgagacgcaaggaccttctagagcacaacaagcttttcagagctttgtaaatgaatgtaatagcatcgagttctcatttgggagctttggagagcatgtgcgagttctctgcgctagagtatcacgaggattagttgccgcaggagaggcgattcgccgttgcttctcttgttgtaaaggatcgacgcatcgctacgctcctcgcgatgacctatctcctgaaggtgcatcgttagcagagactttggctagattcgcagatgatatgggaatagagcaaggtgctgatggaacctacgatattccttgggtagatgattggagaagaggggttcctagtattgaaggagaaggatctgactcgatctatgaaatcatgatgcctatctatgaagttatgaatatggatctagaaacacgaagatcttttgcggtacagcaagggcactatcaggacccaagagcttcagattatgacctcccacgtgctagcgactatgatttgcctagaagcccatatcctactccacctttgccttctagatatcagctacagaatatggatgtagaagcagggttccgtgaggcagtttatgcttcttttgtagcaggaatgtacaattatgtagtgacacagccgcaagagcgtattcccaatagtcagcaggtggaagggattctgcgtgatatgcttaccaacgggtcacagacatttagcgacctgatgaagcgttgggatagagaagtcgatagggaataa

SEQ ID NO: 122 - protein, 590 aa, Chlamydia trachomatis
MSIRGVGGNGNSRIPSHNGDGSNRRSQNTKGNNKVEDRVHSLYSSLSNENRESPYPVVDVSSMIESTPTSGETPRASRGVFSRFQRGLGRVADKVRRAVQCAWGSVSTRRSSATRAVESGSSSRTARGASSGREYSPSAARGLRLMFTDFWRTRVLRQTSPMDVVFGNLDVNEARLMAAYTSECADYLEAHDLAGPDGVAAAREIAQRWEKRVRDLQDKGAAQKLLNDPLGRRTPNYQSKNPGEYTVGNSMFYDGPQVANLQNVDTGFWLDMSNFSDVVLSREIQTGLRARATLEESMPMLENLEERFRRLQETCDAARTEIEESGWTRESASRMGGDETQGPSRAQQAFQSFVNECNSIEFSFGSFGEHVRVLCARVSRGLVAAGEAIRRCFSCCKGSTHRYAPRDDLSPEGASLAETLARFADDMGIEQGADGTYDIPWVDDWRRGVPSIEGEGSDSIYEIMMPIYEVMNMDLETRRSFAVQQGHYQDPRASDYDLPRASDYDLPRSPYPTPPLPSRYQLQNMDVEAGFREAVYASFVAGMYNYVVTQPQERIPNSQQVEGILRDMLTNGSQTFSDLMKRWDREVDRE

SEQ ID NO: 123 - DNA, 1776 bp, Chlamydia trachomatis; identical to SEQ ID NO: 21

SEQ ID NO: 124 - protein, 591 aa, Chlamydia trachomatis; identical to SEQ ID NO: 22

SEQ ID NO: 125 - DNA, 1773 bp, Chlamydia trachomatis
atgagcatcaggggagtaggaggcaacgggaatagtcgaatcccttctcataatggggatggatcgaatcgcagaagtcaaaatacgaagggtaataataaagttgaagatcgagttcattctctatattcatctcttagtaacgaaaatagagaatctccttatccagtagtagacgtcagctctatgatcgagagcaccccaacgagtggagagacgccaagagcttcgcgtggagtgttcagtcgtttccaaagaggtttaggacgagtagctgacaaagtaagacgagctgttcagtgtgcgtggggttcagtctctacaagaagatcgtctgcaacaagagccgtagaatccggatcaagtagtcgtactgctcgtggtgcaagttctgggagggagtattctccttcagcagctagagggctgcgtcttatgttcacagatttctggagaactcgggttttacgccagacctctcctatggatgtagtttttgggaatcttgatgtgaacgaggctcgtttgatggctgcttacacaagtgagtgcgcggattatttagaagcgcacgatttggctggccctgacggggtagcggccgcccgggaaattgctcaaagatgggataaaagagttagagatctacaagataaaggtgctgcacaaaaattattaaatgatcctttaggccgacgaacacctaattatcagagcaaaaatccaggtgagtatactgtagggaattccatgttttacgatggtcctcaggtagcgaatctccagaacgtcgacactggtttttggctggacatgagcaatttctcagacgttgtattatccagagagattcaaacagggcttcgagcacgagctactttggaagaatccatgccgatgttagagaatttagaagagcgttttagacgtttgcaagaaacttgtgatgcggctcgtactgagatagaagaatcgggatggactcgagagtccgcatcaagaatgggaggcgatgagacgcaaggaccttctagagcacaacaagcttttcagagctttgtaaatgaatgtaatagcatcgagttctcatttgggagctttggagagcatgtgcgagttctctgcgctagagtatcacgaggattagttgccgcaggagaggcgattcgccgttgcttctcttgttgtaaaggatcgacgcatcgctacgctcctcgcgatgacctatctcctgaaggtgcatcgttagcagagactttggctagattcgcagatgatatgggaatagagcaaggtgctgatggaacctacgatattccttgggtagatgattggagaagaggggttcctagtattgaaggagaaggatctgactcgatctatgaaatcatgatgcctatctatgaagttatgaatatggatctagaaacacgaagatcttttgcggtacagcaagggcactatcaggacccaagagcttcagattatgacctcccacgtgctagcgactatgatttgcctagaagcccatatcctactccacctttgccttctagatatcagctacagaatatggatgtagaagcagggttccgtgaggcagtttatgcttcttttgtagcaggaatgtacaattatgtagtgacacagccgcaagagcgtattcccaatagtcagcaggtggaagggattctgcgtgatatgcttaccaacgggtcacagacatttagcaacctgatgcagcgttgggatagagaagtcgatagggaataa

SEQ ID NO: 126 - protein, 590 aa, Chlamydia trachomatis
MSIRGVGGNGNSRIPSHNGDGSNRRSQNTKGNNKVEDRVHSLYSSLSNENRESPYPVVDVSSMIESTPTSGETPRASRGVFSRFQRGLGRVADKVRRAVQCAWGSVSTRRSSATRAVESGSSSRTARGASSGREYSPSAARGLRLMFTDFWRTRVLRQTSPMDVVFGNLDVNEARLMAAYTSECADYLEAHDLAGPDGVAAAREIAQRWDKRVRDLQDKGAAQKLLNDPLGRRTPNYQSKNPGEYTVGNSMFYDGPQVANLQNVDTGFWLDMSNFSDVVLSREIQTGLRARATLEESMPMLENLEERFRRLQETCDAARTEIEESGWTRESASRMGGDETQGPSRAQQAFQSFVNECNSIEFSFGSFGEHVRVLCARVSRGLVAAGEAIRRCFSCCKGSTHRYAPRDDLSPEGASLAETLARFADDMGIEQGADGTYDIPWVDDWRRGVPSIEGEGSDSIYEIMMPIYEVMNMDLETRRSFAVQQGHYQDPRASDYDLPRASDYDLPRSPYPTPPLPSRYQLQNMDVEAGFREAVYASFVAGMYNYVVTQPQERIPNSQQVEGILRDMLTNGSQTFSNLMQRWDREVDRE

(19) United States
(12) Patent Application Publication
(10) Pub. No.: US 2009/0022755 A1
(43) Pub. Date: Jan. 22, 2009

(54) VACCINES AGAINST CHLAMYDIAL INFECTION

(75) Inventors: Brenda Barth, Federal Way, WA (US); Ajay Bhatia, Seattle, WA (US); Mark Alderson, Bainbridge Island, WA (US); Jean-Francois L. Maisonneuve, Federal Way, WA (US); Yves Lobet, Rixensart (BE); Florence Bernadette Nozay, Rixensart (BE); Martine Marchand, Rixensart (BE); Pascal Mettens, Rixensart (BE); Yasir A. Skeiky, Silver Spring, MD (US); Peter Probst, Seattle, WA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignees: GLAXOSMITHKLINE BIOLOGICALS SA, Rixensart (BE); CORIXA CORPORATION, Seattle, WA (US)

(21) Appl. No.: 11/909,992

(22) PCT Filed: Mar. 24, 2006
(86) PCT No.: PCT/US06/10793

§ 371 Date: Sep. 28, 2007

Related U.S. Application Data
(60) Provisional application No. 60/667,331, filed on Mar. 31, 2005.

(51) Int. Cl. A61K 39/00; A61P 31/00

(52) U.S. Cl. 424/190.1

(57) ABSTRACT

The present invention relates to compositions comprising proteins or polynucleotides of Chlamydia sp., in particular combinations of proteins or polynucleotides encoding them, and methods for the use of the proteins or polynucleotides in the treatment, prevention and diagnosis of Chlamydia infection.

### FIELD OF THE INVENTION

The present invention relates generally to the treatment or prevention of Chlamydial infection. In particular, the invention is related to compositions of polypeptides comprising a Chlamydia antigen and combinations thereof, and to compositions of polynucleotides encoding a Chlamydia antigen and combinations thereof, and to the use of such compositions for prophylactic or therapeutic treatment of Chlamydial infection.

### BACKGROUND OF THE INVENTION

Chlamydiae are intracellular bacterial pathogens that are responsible for a wide variety of important human and animal infections.

Chlamydia trachomatis is transmitted between human beings through social or sexual contact. A number of Chlamydia trachomatis serovars exist, and although the identification and classification of serovars continues to evolve, at least 18 have been reported to date. Serovars A to C are primarily associated with ocular trachoma, serovars D to K with oculogenital disease and serovars L1 to L3 with lymphogranuloma venereum (LGV) (Brunham, R C et al. J. Nat. Rev. Immunol. 2005 5:149-161).

Chlamydia trachomatis is one of the most common causes of sexually transmitted diseases and can lead to pelvic inflammatory disease (PID), resulting in tubal obstruction and infertility. Chlamydia trachomatis may also play a role in male infertility. In 1990, the cost of treating PID in the US was estimated to be $4 billion. The World Health Organisation estimated that in 1999 over 90 million new cases of sexually transmitted Chlamydia trachomatis occurred worldwide (Global Prevalence and Incidence of Selected Curable Sexually Transmitted Infections, World Health Organisation, Geneva, 2001). Furthermore, ulcerative sexually transmitted diseases such as Chlamydia trachomatis infection are a major risk factor for HIV acquisition (Brunham, R C et al. J. Nat. Rev. Immunol. 2005 5:149-161; Igietseme, J U et al. Expert Rev. Vaccines 2003 2(1):129-146).

Trachoma, due to ocular infection with Chlamydia trachomatis, is the leading cause of preventable blindness worldwide and is estimated to affect 300-500 million people (West, S K Prog. Ret. Eye Res. 2004 23:381-401). Current treatment involves the use of antibiotics such as tetracycline (daily, for a period of 4 to 6 weeks) or azithromycin (single dose). Although effective in combating infection, re-infection generally occurs due to the endemic nature of the infection. Repeated infection over many years leads to scarring of the eyelid, distortion of the lid margin and rubbing of the eye lashes against the cornea (trichiasis). Constant trauma to the cornea is both painful and leads to corneal opacity and blindness (Mabey, D C W et al. The Lancet 2003 362:223-229).

Chlamydia pneumoniae is a major cause of acute respiratory tract infections in humans and is also believed to play a role in the pathogenesis of atherosclerosis and, in particular, coronary heart disease. Individuals with a high titer of antibodies to Chlamydia pneumoniae have been shown to be at least twice as likely to suffer from coronary heart disease as seronegative individuals.

Often chlamydial infection is asymptomatic and subclinical, such that severe and often irreversible complications may present as the first symptoms of genital infection. Infants born from a mother with a genital chlamydial infection may develop pneumonia and Chlamydia trachomatis is considered the most common causative agent of pneumonia during the first six months of life (de la Maza, L M et al. Curr. Opin. Investig. Drugs 2002 3(7):980-986).

Chlamydial infections thus constitute a significant health problem both in developed and developing countries. In light of the public health concerns, and the fact that the cost of current treatments is excessive in many developing countries, the development of vaccines for Chlamydia species has been an important research target. As the genomic make-up of Chlamydia trachomatis is relatively stable, and since the presence of animal reservoirs is negligible, even vaccines with limited efficacy may have a significant impact on the prevalence of infections.

There thus remains a need in the art for improved vaccines and pharmaceutical compositions for the prevention and treatment of Chlamydia infections. There also remains a need in the art for multivalent vaccines for the prevention and treatment of Chlamydia trachomatis infections which are effective against a range of serovars. The present invention fulfills these needs and further provides other related advantages.

### SUMMARY OF THE INVENTION

The present invention relates to compositions comprising antigens of bacterial pathogens of Chlamydia. Such bacterial pathogens include Chlamydia trachomatis, Chlamydia psitacci, Chlamydia pneumonia, and Chlamydia muridarum. The Chlamydia antigens may be derived from any number of serovars within a Chlamydia species.

It should be noted that Chlamydia muridarum was previously known as Chlamydia trachomatis mouse pneumonitis strain (MoPn), both names are still in common use, although they refer to the same bacterium. For consistency, only the name Chlamydia muridarum is used herein.

The present invention is based, in part, on the inventors' discovery that Chlamydia polypeptides possess immunogenic and antigenic properties and can offer protection against chlamydial infection when administered as prophylactic vaccines. Some level of cross reactivity may be seen between antigens of different serovars and species, and therefore Chlamydia antigens are predicted to provide a protective immune response against a species or serovar other than the one from which the antigen was obtained.

More specifically, the inventors have discovered that certain combinations of Chlamydia polypeptides provide a good immune response. Certain combinations of Chlamydia polypeptides have been shown to provide protection against Chlamydia infection in mouse models.

In a specific embodiment, the isolated or purified Chlamydia polypeptides of the invention may be formulated as pharmaceutical compositions for administration into a subject in the prevention and/or treatment of Chlamydia infection. The immunogenicity of the protein composition may be enhanced by the inclusion of an adjuvant.

In a specific embodiment, the isolated or purified Chlamydia polypeptides are administered as combinations of individual antigens, optionally in combination with an adjuvant. Alternatively, the Chlamydia polypeptides are administered in the form of a fusion protein, optionally in combination with an adjuvant.

In another aspect of the invention, isolated or purified polynucleotides are used to produce recombinant polypeptide antigens in vitro. Alternatively, the polynucleotides may be administered into a subject as polynucleotide vaccines to cause antigen expression in the subject, and the subsequent induction of an anti-Chlamydia immune response.

In a further aspect of the invention, certain combinations of Chlamydia polypeptides according to the present invention, immunogenic fragments thereof or polynucleotides encoding them which are derived from a first Chlamydia trachomatis serovar may be administered to a subject for the treatment or prevention of Chlamydia infection from a second Chlamydia trachomatis serovar.

It is also an object of the invention that the polypeptides be used in in vitro assays for detecting humoral antibodies or cell-mediated immunity against Chlamydia for diagnosis of infection or monitoring of disease progression. Alternatively, the polypeptides may be used as immunogens to generate anti-Chlamydia antibodies in a non-human animal. The antibodies can be used to detect the target antigens in vivo and in vitro.

### BRIEF DESCRIPTION OF THE FIGURES

FIG. 1 shows Day 7 bacterial shedding data in Balb/c mice, representing data from three experiments in which groups of 5 Balb/c mice were immunized with the indicated combinations of antigens in AS01B. Graph represents data from three experiments in which groups of 5 Balb/c mice were immunized with the indicated combination of antigens in AS01B adjuvant. UVEB from serovar E formulated with AS01B served as a positive control of protection, and AS01B sham-immunized mice were used as positive control of infection. Progesterone-treated mice were challenged with an intra-vaginal dose of 5×10⁵ IFU of serovar K. Bacterial shedding was quantified by taking swabs on day 7 post infection and determining the IFU using McCoy cells. Data from one back to back experiment were pooled.

FIG. 2 shows Chlamydial shedding in the LGT and chlamydial load post challenge with Chlamydia trachomatis serovar K in the UGT of Balb/c mice immunized with antigen combinations. Graphs represent data from back to back experiments in which groups of 8 balb/c mice were immunized with the indicated combination of antigens in AS01B adjuvant. UVEB from serovar E formulated with AS01B served as a positive control of protection, and AS01B sham-immunized mice were used as control of infection. Progesterone-treated mice were challenged with an intra-vaginal dose of 5×10⁵ IFU of serovar K. Bacterial shedding was quantified by taking swabs on day 7 post challenge and determining the IFU using McCoy cells. Mice were sacrificed 10 days post infection to determine the chlamydial load in the upper genital tract by homogenizing half of the UGT and determining IFU using McCoy cells. It should be noted that the limit of detection was 10 in respect of both of the above plots.

FIG. 3 shows Day 7 bacterial shedding data in C57Bl/6 mice immunized with the indicated combinations of antigens in AS01B. Graphs represent data from back to back experiments in which groups of 8 C57Bl/6 mice were immunized with the indicated combination of antigens in AS01B. UVEB from serovar E formulated with AS01B served as a positive control of protection, and AS01B sham-immunized mice were used as control of infection. Progesterone-treated mice were challenged with an intra-vaginal dose of 5×10⁵ IFU of serovar K. Bacterial shedding was quantified by taking swabs on day 7 post challenge and determining the IFU using McCoy cells.

FIG. 4 shows Day 7 bacterial shedding data in Balb/c mice immunized with the indicated combinations of antigens in AS01B. Graphs represent pooled data from 5 experiments in which groups of 5-8 Balb/c mice were immunized with the indicated combination of antigens in AS01B. UVEB from serovar E formulated with AS01B served as a positive control of protection, and AS01B sham-immunized mice were used as control of infection. Progesterone-treated mice were challenged with an intra-vaginal dose of 5×10⁵ IFU of serovar K. Bacterial shedding was quantified by taking swabs on day 7 post challenge and determining the IFU using McCoy cells.

FIG. 5 shows Day 7 bacterial shedding data in C57Bl/6 mice immunized with the indicated combinations of antigens in AS01B. Graphs represent pooled data from 3 experiments in which groups of 5-8 C57Bl/6 mice were immunized with the indicated combination of antigens in AS01B. UVEB from serovar E formulated with AS01B served as a positive control of protection, and AS01B sham-immunized mice were used as control of infection. Progesterone-treated mice were challenged with an intra-vaginal dose of 5×10⁵ IFU of serovar K. Bacterial shedding was quantified by taking swabs on day 7 post challenge and determining the IFU using McCoy cells.

FIG. 6 shows the sequence alignment for Ct-089 from Chlamydia trachomatis serovar E with Ct-089 from a range of other Chlamydia trachomatis serovars.

FIGS. 7a and 7b show the sequence alignment for Ct-858 from Chlamydia trachomatis serovar E with Ct-858 from a range of other Chlamydia trachomatis serovars.

FIGS. 8a and 8b show the sequence alignment for Ct-875 from Chlamydia trachomatis serovar E with Ct-875 from a range of other Chlamydia trachomatis serovars.

FIG. 9 shows the results of an amino acid sequence identity comparison of Ct-089 from Chlamydia trachomatis serovar E with Ct-089 from a range of other Chlamydia trachomatis serovars.

FIG. 10 shows the results of an amino acid sequence identity comparison of Ct-858 from Chlamydia trachomatis serovar E with Ct-858 from a range of other Chlamydia trachomatis serovars.

FIG. 11 shows the results of an amino acid sequence identity comparison of Ct-875 from Chlamydia trachomatis serovar E with Ct-875 from a range of other Chlamydia trachomatis serovars.

FIG. 12 shows the sequence alignment for Ct-089 from Chlamydia trachomatis serovar E with equivalent proteins from other Chlamydia trachomatis serovars and Chlamydia species.

FIG. 13 shows the sequence alignment for Ct-858 from Chlamydia trachomatis serovar E with equivalent proteins from other Chlamydia trachomatis serovars and Chlamydia species.

FIG. 14 shows the sequence alignment for Ct-875 from Chlamydia trachomatis serovar E with equivalent proteins from other Chlamydia trachomatis serovars and Chlamydia species.

FIG. 15 shows swab results taken from Chlamydia trachomatis serovar E Ct-089, Ct-858 and Ct-875 immunised mice four days after challenge from Chlamydia trachomatis serovars D, K or J. UV EB in each case are derived from the same servovar used for challenge (i.e. J, D and K respectively). Both UV EB and the combination treatment are performed in the presence of the adjuvant (i.e. AS01B).

FIG. 16 shows swab results taken from Chlamydia trachomatis serovar E Ct-089, Ct-858 and Ct-875 immunised mice seven days after challenge from Chlamydia trachomatis serovars D, K or J. UV EB in each case are derived from the same servovar used for challenge (i.e. J, D and K respectively). Both UV EB and the combination treatment are performed in the presence of the adjuvant (i.e. AS01B).

FIG. 17 shows colonisation of the UGT from Chlamydia trachomatis serovar E Ct-089, Ct-858 and Ct-875 immunised mice ten days after challenge from Chlamydia trachomatis serovars D, K or J. UV EB in each case are derived from the same serovar used for challenge (i.e. J, D and K respectively). Both UV EB and the combination treatment are performed in the presence of the adjuvant (i.e. AS01B).

FIG. 18 shows swab results taken from Chlamydia trachomatis serovar E Ct-089, Ct-858 and Ct-875 immunised mice four days after challenge from Chlamydia trachomatis serovars K or J. UV EB in each case are derived from the same servovar used for challenge (i.e. J and K respectively). Both UV EB and the combination treatment are performed in the presence of the adjuvant (i.e. AS01B).

FIG. 19 shows swab results taken from Chlamydia trachomatis serovar E Ct-089, Ct-858 and Ct-875 immunised mice seven days after challenge from Chlamydia trachomatis serovars K or J. UV EB in each case are derived from the same servovar used for challenge (i.e. J and K respectively). Both UV EB and the combination treatment are performed in the presence of the adjuvant (i.e. AS01B).

FIG. 20 shows colonisation of the UGT from Chlamydia trachomatis serovar E Ct-089, Ct-858 and Ct-875 immunised mice ten days after challenge from Chlamydia trachomatis serovars K or J. UV EB in each case are derived from the same serovar used for challenge (i.e. J and K respectively). Both UV EB and the combination treatment are performed in the presence of the adjuvant (i.e. AS01B).

FIG. 21 shows the proportion of CD4 responders (for a signal to noise S/N cut-off of at least 2:1) to stimulation with a range of antigens for a number of seropositive subject groups.

FIG. 22 shows the proportion of CD4 responders (for a signal to noise cut-off of at least 3:1) to stimulation with a range of antigens for a number of seropositive subject groups.

FIG. 23 shows the proportion of CD4 responders (for a signal to noise cut-off of at least 2:1) to stimulation with a range of antigens and combinations thereof for a number of seropositive subject groups.

### DESCRIPTION OF THE SPECIFIC EMBODIMENTS

The present invention relates to compositions comprising combinations of antigens useful for the diagnosis, prevention and treatment of Chlamydia infection, polynucleotides encoding such antigens, and methods for their use. The antigens of the present invention are polypeptides of Chlamydia antigens and immunogenic fragments thereof.

In particular, compositions of the present invention may comprise a combination of two or more Chlamydia proteins or immunogenic fragments thereof. Such proteins may be selected from Swib (also known as Ct-460), Momp (major outer membrane protein, also known as Ct-681), Ct-858, Ct-875, Ct-622, Ct-089 (also known as CopN), passenger domain of PmpG (PmpGpd, also known as Ct-871) and passenger domain of PmpD (PmpDpd, also known as Ct-812).

For example, the composition of the present invention may comprise Ct-089 and Ct-858 or immunogenic fragments thereof and optionally further antigens which may be selected for example from Momp, Ct-875, Ct-622, PmpGpd and PmpDpd. In a further example, the composition of the present invention may comprise Ct-875 and Ct-858 or immunogenic fragments thereof and optionally further antigens which may be selected for example from Momp, Ct-622, Ct-089, PmpGpd and PmpDpd.

For example the composition of the present invention may comprise one of the following combinations of Chlamydia polypeptides or immunogenic fragments thereof:
1. Momp, PmpDpd, Ct-858, Ct-089, Swib
1′. PmpDpd, Ct-858, Ct-089, Swib
2. Momp, PmpDpd, Ct-858, Ct-622, Ct-089, Swib
3. Momp, PmpDpd, Ct-858, PmpGpd, Ct-622, Ct-089
4. Ct-858, Ct-875, Ct-622, Ct-089
5. Ct-858, Ct-875, Ct-089
5′. PmpDpd, Ct-858, Ct-875, Ct-089
6. Momp, PmpD, Ct-858, PmpGpd, Ct-089

All of the above combinations comprise Ct-089 and Ct-858.

In a further set of examples, the composition of the present invention comprises one of the following combinations, provided that all of the combinations comprise Ct-089 and Ct-858:
1a. All five of: Momp, PmpDpd, Ct-858, PmpGpd and Ct-089
1′a. Three out of: PmpDpd, Ct-858, Ct-089, Swib
2a. Five out of: Momp, PmpDpd, Ct-858, Ct-622, Ct-089 and Swib
3a. Five out of: Momp, PmpDpd, Ct-858, PmpGpd, Ct-622 and Ct-089
4a. Three out of: Ct-858, Ct-875, Ct-622 and Ct-089
5a. Two out of: Ct-858, Ct-875 and Ct-089
5′a. Three out of: PmpDpd, Ct-858, Ct-875, Ct-089
6a. Four out of: Momp, PmpD, Ct-858, PmpGpd and Ct-089 or alternatively
1a″. Five out of: Swib, Momp, PmpDpd, Ct-858, PmpGpd and Ct-089

Other example compositions of the present invention may comprise one of the following combinations of Chlamydia polypeptides or immunogenic fragments thereof:
1b. Momp, PmpDpd, Ct-858, Ct-875, Swib, Ct-089
1b′. PmpDpd, Ct-858, Ct-875, Swib, Ct-089
2b. Momp, PmpDpd, Ct-858, Ct-622, Ct-875, Swib, Ct-089
3b. Momp, PmpDpd, Ct-858, PmpGpd, Ct-622, Ct-875, Ct-089
4b. Ct-858, Ct-875
5b. Momp, Ct-858, Ct-875, Ct-089
5b′. Momp, Ct-858, Ct-875
6b. Momp, PmpD, Ct-858, PmpGpd, Ct-875, Ct-089

All of the above combinations comprise Ct-875 and Ct-858.

In a further set of examples, the composition of the present invention comprises one of the following combinations, provided that all of the combinations comprise Ct-875 and Ct-858:
1c. Five out of: Swib, Momp, PmpDpd, Ct-858, PmpGpd and Ct-875
1c′. Three out of: PmpDpd, Ct-858, Ct-0875, Swib
2c. Five out of: Momp, PmpDpd, Ct-858, Ct-622, Ct-875 and Swib
3c. Five out of: Momp, PmpDpd, Ct-858, PmpGpd, Ct-622 and Ct-875
4c. Three out of: Ct-858, Ct-875, Ct-622 and Ct-089
5c′. Three out of: PmpDpd, Ct-858, Ct-875, Ct-089
6c. Four out of: Momp, PmpD, Ct-858, PmpGpd and Ct-875

The compositions according to the invention comprise two or more Chlamydia proteins or immunogenic fragments, for example 3, 4, 5, 6, 7, 8, 9 or 10 proteins or immunogenic fragments. For a composition comprising each of the combinations listed above under numbers 1-6, 1a-6a, 1b-6b and 1c-6c (e.g. 1-6 and 1a-6a) the combination may include further Chlamydia antigens, for example one further Chlamydia antigen, or it may contain no more Chlamydia antigens than those listed. For example, composition 1a″ may contain only five antigens which are a combination of those Chlamydia antigens as listed and no other antigens, or composition 1a″ may comprise a combination of five of the Chlamydia antigens as listed (such as all six antigens listed, or five of the six antigens listed plus one other Chlamydia antigen), and so forth for compositions 2-6, 2a-6a, 1b-6b and 1c-6c (e.g. 2-6 and 2a-6a).

It will be evident that in the case of the passenger domains of PmpD and PmpG, these may be present in the context of a larger portion of the PmpD or PmpG antigen or polynucleotide, for example full length PmpD or PmpG or a fragment thereof, provided that the fragment comprises the passenger domain.

The Momp and Swib proteins or immunogenic fragments may be for example from Chlamydia trachomatis, or they may be from other species of Chlamydia. The antigens above designated “Ct” may be Chlamydia trachomatis proteins or immunogenic fragments, or, where possible, they may be the equivalent proteins from different species of Chlamydia (i.e. a Chlamydia species other than Chlamydia trachomatis). In one example, all of the antigens in the composition according to the invention are from Chlamydia trachomatis.

Compositions of the present invention may alternatively comprise polynucleotides encoding the combination of two or more Chlamydia proteins or immunogenic fragments which may be selected from Swib (also known as Ct-460), Momp (major outer membrane protein also known as Ct-681), Ct-858, Ct-875, Ct-622, Ct-089, passenger domain of PmpG (PmpGpd, also known as Ct-871) and passenger domain of PmpD (PmpDpd, also known as Ct-812), for example the combinations of antigens listed above as 1-6, 1a-6a, 1b-6b and 1c-6c (e.g. 1-6). The compositions of polynucleotides according to the invention include those which encode the combinations of antigens according to the invention as described herein (for example Ct-858 and Ct-875). The polynucleotides encoding the different antigens may be present as separate nucleic acids or they may be present together in a single nucleic acid, or a combination of separate and combined nucleic acids.

The following provides polynucleotide and polypeptide sequences for some of the antigens, which may be used in the compositions of the invention and which have been listed above.

### BRIEF DESCRIPTION OF SEQUENCE IDENTIFIERS

SEQ ID NO:1 is the cDNA sequence of Ct-460, also known as Swib from Chlamydia trachomatis, serovar LGVII (serovar LGVII is also referred to as serovar LII).

SEQ ID NO:2 is the protein sequence of Ct-460, also known as Swib from Chlamydia trachomatis, serovar LGVII, which protein is encoded by SEQ ID NO:1.

SEQ ID NO:3 is the cDNA sequence of the Chlamydia antigen known as Major Outer Membrane Protein (Momp) from Chlamydia trachomatis, serovar F.

SEQ ID NO:4 is the protein sequence of the chlamydia antigen known as Major Outer Membrane Protein (Momp) from Chlamydia trachomatis, serovar F, which protein is encoded by SEQ ID NO:3.

SEQ ID NO:5 is the cDNA sequence of Ct-858 from Chlamydia trachomatis, serovar E.

SEQ ID NO:6 is the protein sequence of Ct-858 Chlamydia trachomatis, serovar E, which protein is encoded by SEQ ID NO:5.

SEQ ID NO:7 is the cDNA sequence of Ct-875 from Chlamydia trachomatis, serovar E.

SEQ ID NO: 8 is the protein sequence of Ct-875 from Chlamydia trachomatis, serovar E, which protein is encoded by SEQ ID NO: 7.

SEQ ID NO: 9 is the cDNA sequence of Ct-622 from Chlamydia trachomatis, serovar E.

SEQ ID NO: 10 is the protein sequence of Ct-622 from Chlamydia trachomatis, serovar E, which protein is encoded by SEQ ID NO: 9.

SEQ ID NO: 11 is the cDNA sequence of the passenger domain of PmpG also known as Ct-871 from Chlamydia trachomatis, serovar LGVII.

SEQ ID NO: 12 is the protein sequence of the passenger domain of PmpG, also known as Ct-871 from Chlamydia trachomatis, serovar LGVII, which protein is encoded by SEQ ID NO: 11.

SEQ ID NO: 13 is the cDNA sequence of the passenger domain of PmpD, also known as Ct-812, from Chlamydia trachomatis, serovar LGVII.

SEQ ID NO: 14 is the protein sequence of the passenger domain of PmpD, also known as Ct-812, from Chlamydia trachomatis, serovar LGVII, which protein is encoded by SEQ ID NO: 13.

SEQ ID NO: 15 is the cDNA sequence of the Ct-089 from Chlamydia trachomatis, serovar E.

SEQ ID NO: 16 is the protein sequence of Ct-089 from Chlamydia trachomatis, serovar E, which protein is encoded by SEQ ID NO: 15.

SEQ ID NO: 17 is the cDNA sequence of the chlamydia antigen known as Major Outer Membrane Protein (Momp) from Chlamydia psitacci.

SEQ ID NO: 18 is the protein sequence of the chlamydia antigen known as Major Outer Membrane Protein (Momp) from Chlamydia psitacci, which protein is encoded by SEQ ID NO: 17.

SEQ ID NO: 19 is the cDNA sequence of the Chlamydia antigen known as Major Outer Membrane Protein (Momp) from Chlamydia pneumoniae.

SEQ ID NO: 20 is the protein sequence of the chlamydia antigen known as Major Outer Membrane Protein (Momp) from Chlamydia pneumoniae, which protein is encoded by SEQ ID NO: 19.

SEQ ID NO: 21 is the cDNA sequence of Ct-875 from Chlamydia trachomatis, serovar D.

SEQ ID NO: 22 is the protein sequence of Ct-875 from Chlamydia trachomatis, serovar D which protein is encoded by SEQ ID NO: 21.

SEQ ID NO: 23 is the cDNA sequence of Ct-875 from Chlamydia muridarum.

SEQ ID NO: 24 is the protein sequence of Ct-875 from Chlamydia muridarum, which protein is encoded by SEQ ID NO:23.

SEQ ID NO: 25 is the cDNA sequence of Ct-875 from Chlamydia psitacci

SEQ ID NO: 26 is the protein sequence of Ct-875 from Chlamydia psitacci, which protein is encoded by SEQ ID NO:25.

SEQ ID NO: 27 is the cDNA sequence PmpG also known as Ct-871 from Chlamydia trachomatis, serovar D.

SEQ ID NO: 28 is the protein sequence of PmpG, also known as Ct-871 from Chlamydia trachomatis, serovar D, which protein is encoded by SEQ ID NO:27.

SEQ ID NO: 29 is the cDNA sequence PmpG also known as Ct-871 from Chlamydia muridarum.

SEQ ID NO: 30 is the protein sequence of PmpG, also known as Ct-871 from Chlamydia muridarum, which protein is encoded by SEQ ID NO:29.

SEQ ID NO: 31 is the cDNA sequence PmpG also known as Ct-871 from Chlamydia psitacci.

SEQ ID NO: 32 is the protein sequence of PmpG, also known as Ct-871 from Chlamydia psitacci, which protein is encoded by SEQ ID NO:31.

SEQ ID NO: 33 is the cDNA sequence of Ct-858 from Chlamydia trachomatis, serovar D.

SEQ ID NO: 34 is the protein sequence of Ct-858 Chlamydia trachomatis, serovar D, which protein is encoded by SEQ ID NO: 33.

SEQ ID NO: 35 is the cDNA sequence of Ct-858 from Chlamydia muridarum.

SEQ ID NO: 36 is the protein sequence of Ct-858 Chlamydia muridarum, which protein is encoded by SEQ ID NO: 35.

SEQ ID NO: 37 is the cDNA sequence of Ct-858 from Chlamydia psitacci.

SEQ ID NO: 38 is the protein sequence of Ct-858 Chlamydia psitacci, which protein is encoded by SEQ ID NO: 37.

SEQ ID NO: 39 is the cDNA sequence of Ct-858 from Chlamydia pneumoniae.

SEQ ID NO: 40 is the protein sequence of Ct-858 Chlamydia pneumoniae, which protein is encoded by SEQ ID NO: 39.

SEQ ID NO: 41 is the cDNA sequence of PmpD, also known as Ct-812, from Chlamydia trachomatis, serovar D.

SEQ ID NO: 42 is the protein sequence of PmpD, also known as Ct-812, from Chlamydia trachomatis, serovar D, which protein is encoded by SEQ ID NO: 41. The passenger domain spans amino acids 31 to 1203.

SEQ ID NO: 43 is the cDNA sequence of PmpD, also known as Ct-812, from Chlamydia muridarum.

SEQ ID NO: 44 is the protein sequence of PmpD, also known as Ct-812, from Chlamydia muridarum, which protein is encoded by SEQ ID NO: 43.

SEQ ID NO: 45 is the cDNA sequence of PmpD, also known as Ct-812, from Chlamydia psitacci.

SEQ ID NO: 46 is the protein sequence of PmpD, also known as Ct-812, from Chlamydia psitacci, which protein is encoded by SEQ ID NO: 45.

SEQ ID NO: 47 is the cDNA sequence of the Chlamydia antigen known as Major Outer Membrane Protein (Momp), also known as Ct-681 from Chlamydia trachomatis, serovar LGVII.

SEQ ID NO: 48 is the protein sequence of the chlamydia antigen known as Major Outer Membrane Protein (Momp), also known as Ct-681 from Chlamydia trachomatis, serovar LGVII, which protein is encoded by SEQ ID NO: 47.

SEQ ID NO: 49 is the cDNA sequence of the Chlamydia antigen known as Major Outer Membrane Protein (Momp), also known as Ct-681 from Chlamydia trachomatis, serovar J.

SEQ ID NO: 50 is the protein sequence of the chlamydia antigen known as Major Outer Membrane Protein (Momp), also known as Ct-681 from Chlamydia trachomatis, serovar J, which protein is encoded by SEQ ID NO: 49.

SEQ ID NO: 51 is the cDNA sequence of the Chlamydia antigen known as Major Outer Membrane Protein (Momp), also known as Ct-681 from Chlamydia trachomatis, serovar H.

SEQ ID NO: 52 is the protein sequence of the chlamydia antigen known as Major Outer Membrane Protein (Momp), also known as Ct-681 from Chlamydia trachomatis, serovar H, which protein is encoded by SEQ ID NO: 51.

SEQ ID NO: 53 is the cDNA sequence of the Chlamydia antigen known as Major Outer Membrane Protein (Momp), also known as Ct-681 from Chlamydia trachomatis, serovar E.

SEQ ID NO: 54 is the protein sequence of the chlamydia antigen known as Major Outer Membrane Protein (Momp), also known as Ct-681 from Chlamydia trachomatis, serovar E, which protein is encoded by SEQ ID NO: 53.

SEQ ID NO: 55 is the cDNA sequence of the Chlamydia antigen known as Major Outer Membrane Protein (Momp), also known as Ct-681 from Chlamydia trachomatis, serovar D.

SEQ ID NO: 56 is the protein sequence of the chlamydia antigen known as Major Outer Membrane Protein (Momp), also known as Ct-681 from Chlamydia trachomatis, serovar D, which protein is encoded by SEQ ID NO: 55.

SEQ ID NO: 57 is the cDNA sequence of Ct-622 from Chlamydia trachomatis, serovar D.

SEQ ID NO: 58 is the protein sequence of Ct-622 from Chlamydia trachomatis, serovar D, which protein is encoded by SEQ ID NO: 57.

SEQ ID NO: 59 is the cDNA sequence of Ct-622 from Chlamydia psitacci.

SEQ ID NO: 60 is the protein sequence of Ct-622 from Chlamydia psitacci, which protein is encoded by SEQ ID NO: 59.

SEQ ID NO: 61 is the cDNA sequence of Ct-622 from Chlamydia pneumoniae.

SEQ ID NO: 62 is the protein sequence of Ct-622 from Chlamydia pneumoniae, which protein is encoded by SEQ ID NO: 61.

SEQ ID NO: 63 is the cDNA sequence of Ct-460, also known as Swib from Chlamydia trachomatis, serovar D.

SEQ ID NO: 64 is the protein sequence of Ct-460, also known as Swib from Chlamydia trachomatis, serovar D, which protein is encoded by SEQ ID NO: 63.

SEQ ID NO: 65 is the cDNA sequence of Ct-460, also known as Swib from Chlamydia muridarum.

SEQ ID NO: 66 is the protein sequence of Ct-460, also known as Swib from Chlamydia muridarum, which protein is encoded by SEQ ID NO: 65.

SEQ ID NO: 67 is the cDNA sequence of Ct-460, also known as Swib from Chlamydia psitacci.

SEQ ID NO: 68 is the protein sequence of Ct-460, also known as Swib from Chlamydia psitacci, which protein is encoded by SEQ ID NO: 67.

SEQ ID NO: 69 is the cDNA sequence of Ct-460, also known as Swib from Chlamydia pneumoniae.

SEQ ID NO: 70 is the protein sequence of Ct-460, also known as Swib from Chlamydia pneumoniae, which protein is encoded by SEQ ID NO:69.

SEQ ID NO: 71 is the cDNA sequence of the Ct-089 from Chlamydia trachomatis, serovar D.

SEQ ID NO: 72 is the protein sequence of Ct-089 from Chlamydia trachomatis, serovar D, which protein is encoded by SEQ ID NO: 71.

SEQ ID NO: 73 is the cDNA sequence of the Ct-089 from Chlamydia muridarum.

SEQ ID NO: 74 is the protein sequence of Ct-089 from Chlamydia muridarum, which protein is encoded by SEQ ID NO: 73.

SEQ ID NO: 75 is the cDNA sequence of the Ct-089 from Chlamydia psitacci.

SEQ ID NO: 76 is the protein sequence of Ct-089 from Chlamydia psitacci, which protein is encoded by SEQ ID NO: 75.

SEQ ID NO: 77 is the cDNA sequence of the Ct-089 from Chlamydia pneumoniae.

SEQ ID NO: 78 is the protein sequence of Ct-089 from Chlamydia pneumoniae, which protein is encoded by SEQ ID NO: 77.

SEQ ID NO: 79 is the cDNA sequence of the Ct-089 from Chlamydia trachomatis, serovar A.

SEQ ID NO: 80 is the protein sequence of Ct-089 from Chlamydia trachomatis, serovar A, which protein is encoded by SEQ ID NO: 79.

SEQ ID NO: 81 is the cDNA sequence of the Ct-089 from Chlamydia trachomatis, serovar B.

SEQ ID NO: 82 is the protein sequence of Ct-089 from Chlamydia trachomatis, serovar B, which protein is encoded by SEQ ID NO: 81.

SEQ ID NO: 83 is the cDNA sequence of the Ct-089 from Chlamydia trachomatis, serovar G.

SEQ ID NO: 84 is the protein sequence of Ct-089 from Chlamydia trachomatis, serovar G, which protein is encoded by SEQ ID NO: 83.

SEQ ID NO: 85 is the cDNA sequence of the Ct-089 from Chlamydia trachomatis, serovar H.

SEQ ID NO: 86 is the protein sequence of Ct-089 from Chlamydia trachomatis, serovar H, which protein is encoded by SEQ ID NO: 85.

SEQ ID NO: 87 is the cDNA sequence of the Ct-089 from Chlamydia trachomatis, serovar I.

SEQ ID NO: 88 is the protein sequence of Ct-089 from Chlamydia trachomatis, serovar I, which protein is encoded by SEQ ID NO: 87.

SEQ ID NO: 89 is the cDNA sequence of the Ct-089 from Chlamydia trachomatis, serovar J.

SEQ ID NO: 90 is the protein sequence of Ct-089 from Chlamydia trachomatis, serovar J, which protein is encoded by SEQ ID NO: 89.

SEQ ID NO: 91 is the cDNA sequence of the Ct-089 from Chlamydia trachomatis, serovar K.

SEQ ID NO: 92 is the protein sequence of Ct-089 from Chlamydia trachomatis, serovar K, which protein is encoded by SEQ ID NO: 91.

SEQ ID NO: 93 is the cDNA sequence of the Ct-089 from Chlamydia trachomatis, serovar L2.

SEQ ID NO: 94 is the protein sequence of Ct-089 from Chlamydia trachomatis, serovar L2, which protein is encoded by SEQ ID NO: 93.

SEQ ID NO: 95 is the cDNA sequence of the Ct-858 from Chlamydia trachomatis, serovar A.

SEQ ID NO: 96 is the protein sequence of Ct-858 from Chlamydia trachomatis, serovar A, which protein is encoded by SEQ ID NO: 95.

SEQ ID NO: 97 is the cDNA sequence of the Ct-858 from Chlamydia trachomatis, serovar B.

SEQ ID NO: 98 is the protein sequence of Ct-858 from Chlamydia trachomatis, serovar B, which protein is encoded by SEQ ID NO: 97.

SEQ ID NO: 99 is the cDNA sequence of the Ct-858 from Chlamydia trachomatis, serovar G.

SEQ ID NO: 100 is the protein sequence of Ct-858 from Chlamydia trachomatis, serovar G, which protein is encoded by SEQ ID NO: 99.

SEQ ID NO: 101 is the cDNA sequence of the Ct-858 from Chlamydia trachomatis, serovar H.

SEQ ID NO: 102 is the protein sequence of Ct-858 from Chlamydia trachomatis, serovar H, which protein is encoded by SEQ ID NO: 101.

SEQ ID NO: 103 is the cDNA sequence of the Ct-858 from Chlamydia trachomatis, serovar I.

SEQ ID NO: 104 is the protein sequence of Ct-858 from Chlamydia trachomatis, serovar I, which protein is encoded by SEQ ID NO: 103.

SEQ ID NO: 105 is the cDNA sequence of the Ct-858 from Chlamydia trachomatis, serovar J.

SEQ ID NO: 106 is the protein sequence of Ct-858 from Chlamydia trachomatis, serovar J, which protein is encoded by SEQ ID NO: 105.

SEQ ID NO: 107 is the cDNA sequence of the Ct-858 from Chlamydia trachomatis, serovar K.

SEQ ID NO: 108 is the protein sequence of Ct-858 from Chlamydia trachomatis, serovar K, which protein is encoded by SEQ ID NO: 107.

SEQ ID NO: 109 is the cDNA sequence of the Ct-858 from Chlamydia trachomatis, serovar L2.

SEQ ID NO: 110 is the protein sequence of Ct-858 from Chlamydia trachomatis, serovar L2, which protein is encoded by SEQ ID NO: 109.

SEQ ID NO: 111 is the cDNA sequence of the Ct-875 from Chlamydia trachomatis, serovar A.

SEQ ID NO: 112 is the protein sequence of Ct-875 from Chlamydia trachomatis, serovar A, which protein is encoded by SEQ ID NO: 111.

SEQ ID NO: 113 is the cDNA sequence of the Ct-875 from Chlamydia trachomatis, serovar B.

SEQ ID NO: 114 is the protein sequence of Ct-875 from Chlamydia trachomatis, serovar B, which protein is encoded by SEQ ID NO: 113.

SEQ ID NO: 115 is the cDNA sequence of the Ct-875 from Chlamydia trachomatis, serovar G.

SEQ ID NO: 116 is the protein sequence of Ct-875 from Chlamydia trachomatis, serovar G, which protein is encoded by SEQ ID NO: 115.

SEQ ID NO: 117 is the cDNA sequence of the Ct-875 from Chlamydia trachomatis, serovar H.

SEQ ID NO: 118 is the protein sequence of Ct-875 from Chlamydia trachomatis, serovar H, which protein is encoded by SEQ ID NO: 117.

SEQ ID NO: 119 is the cDNA sequence of the Ct-875 from Chlamydia trachomatis, serovar I.

SEQ ID NO: 120 is the protein sequence of Ct-875 from Chlamydia trachomatis, serovar I, which protein is encoded by SEQ ID NO: 119.

SEQ ID NO: 121 is the cDNA sequence of the Ct-875 from Chlamydia trachomatis, serovar J.

SEQ ID NO: 122 is the protein sequence of Ct-875 from Chlamydia trachomatis, serovar J, which protein is encoded by SEQ ID NO: 121.

SEQ ID NO: 123 is the cDNA sequence of the CT875 from Chlamydia trachomatis, serovar K.

SEQ ID NO: 124 is the protein sequence of Ct-875 from Chlamydia trachomatis, serovar K, which protein is encoded by SEQ ID NO: 123.

SEQ ID NO: 125 is the cDNA sequence of the Ct-875 from Chlamydia trachomatis, serovar L2.

SEQ ID NO: 126 is the protein sequence of Ct-875 from Chlamydia trachomatis, serovar L2, which protein is encoded by SEQ ID NO: 125.

Certain of the above sequences and other related Chlamydia polypeptides and polynucleotides from a number of serovars are known and available in the art. Further related sequences can be found in issued U.S. Pat. Nos. 6,447,779, 6,166,177, 6,565,856, 6,555,115, 6,432,916, and 6,448,234 and are also disclosed in U.S. patent application Ser. Nos. 10/197,220, 10/762,058 and 10/872,155, each of which is herein incorporated by reference.

The sequence of Ct-089 from serovar D and the potential application of this protein as an antigen has been publicly disclosed, for example in WO02/08267 (Corixa Corporation). The sequence of Ct-089 from serovar L2 was disclosed in WO99/28475 (Genset). The role of CopN (also known as Ct-089) as a putative exported regulator of type III protein secretion systems is discussed in Fields, K A and Hackstadt, T Mol. Microbiol. 2000 38(5):1048-1060.

The sequences of Ct-858 and Ct-875 from serovar D are available from the Swiss-Prot database, primary accession numbers O84866 and O84883 respectively. For further information see Stephens, R S et al. Science 1998 282:754-759.

The use of Ct-858 as an antigen is disclosed, for example, in WO02/08267 (Corixa Corporation).

The sequence of Ct-875 from serovar E (incorporating a His-tag) and its use as an antigen is disclosed, for example, in US 20040137007. However, the document incorrectly refers to Sequence 139 as being Ct-875, when it is in fact Sequence 140 therein.

Individuals who have been exposed to Chlamydia trachomatis have been shown to develop some degree of natural immunity to reinfection, at least in the case of the same serovar (Katz, B P et al. Sex. Transm. Dis. 1987 14:160-164), although the extent of protection may depend upon the time elapsed since the prior infection occurred. Age has also been shown to be important in the duration of infection, with older individuals demonstrating a shorter duration of infection by ocular Chlamydia trachomatis (Bailey, R et al. Epidemiol. Infect. 1999 123:479-486), again suggesting the existence of adaptive immunological protection. It has been suggested that the use of antibiotics may in fact hamper the development of natural immunity to Chlamydia trachomatis (Brunham, R C et al. J. Nat. Rev. Immunol 2005 5:149-161).

The major outer membrane protein (Momp) constitutes approximately 60% of the protein mass of the bacterial outer membrane and is believed to be important in the determination of serotype specificity. The amino acid sequence contains four regions which are externally exposed and in which the majority of sequence variations occur. Of the ca. 400 amino acids in the Momp sequence, up to 70 amino acids differ between Momp from different serovars. Particularly surprising is the finding that serovar grouping based on amino acid sequence identity does not correspond to the serovar grouping based on disease state (i.e. ocular, oculogenital and LGV) (Stothard, D R et al. Infect Immun. 1998 66(8):3618-3625). Similarly, nucleotide sequence identity comparisons for the ompA gene which encodes Momp do not correspond to disease states (Meijer, A et al. J. Bateriol. 1999 181(15):4469-4475; Lysen, M et al. J. Clin. Microbiol. 2004 42(4):1641-1647). Monoclonal antibodies for Momp are effective in culture and in some animal models, however, protection can be limited and is generally serovar specific.

Mice immunised subcutaneously or orally with a monoclonal anti-idiotypic body to the exoglycolipid antigen developed a protective response to serovar C, though remained susceptible to challenge with serovar K (Whittum-Hudson, J A et al. Nat. Med. 1996 2(10):1116-1121).

One protein which has been disclosed to date and which shows a high level of sequence homology among different serovars, namely class I accessible protein-1 (referred to as Cap1, or Ct-529), such proteins have potential use in the development of vaccines which stimulate protection against more than one serovar (Fling, S P et al. PNAS 2001 98(3):11160-1165). However, in addition to the requirement for high levels of sequence homology between serovars, proteins of use in vaccines must also elicit sufficient immune response.

Surprisingly, it has been found that Chlamydia trachomatis proteins Ct-089, Ct-858 and Ct-875 in particular are both highly antigenic and have a high degree of sequence identity across the different Chlamydia trachomatis serovars. There is particularly high conservation in the region of the predicted epitopes. In light of this finding, the possibility exists for the development of Chlamydia vaccines which are effective against a broad range of Chlamydia trachomatis serovars (i.e. which may be of use in cross-protection).

According to this aspect of the present invention there is provided the use of one or more Chlamydial proteins, immunogenic fragments thereof or polynucleotides encoding them, selected from the list consisting of Ct-089, Ct-858 and Ct-875, and which are derived from a first Chlamydia trachomatis serovar, in the manufacture of a vaccine for the treatment or prevention of Chlamydial infection by a second Chlamydia trachomatis serovar.

In a further aspect of the present invention there is provided a method for the treatment or prevention of Chlamydial infection by a second Chlamydia trachomatis serovar, comprising the administration of a vaccine comprising one or more Chlamydial proteins, immunogenic fragments thereof or polynucleotides encoding them, selected from the list consisting of Ct-089, Ct-858 and Ct-875, and which are derived from a first Chlamydia trachomatis serovar.

In one embodiment of the invention the cross-protection vaccine comprises one protein, immunogenic fragment thereof or polynucleotide encoding them, selected from the list consisting of Ct-089, Ct-858 and Ct-875. Vaccines which comprise only one protein, immunogenic fragment thereof or polynucleotide encoding them, selected from the list consisting of Ct-089, Ct-858 and Ct-875 will suitably further comprise at least one additional Chlamydial antigen (for example 1 or 2 additional antigens).

In a second embodiment of the invention the cross-protection vaccine comprises two proteins, immunogenic fragments thereof or polynucleotides encoding them, selected from the list consisting of Ct-089, Ct-858 and Ct-875. For example: Ct-089 and Ct-858; Ct-089 and Ct-875; or Ct-858 and Ct-875.

In a third embodiment of the invention the cross-protection vaccine comprises Ct-089, Ct-858 and Ct-875, immunogenic fragments thereof or polynucleotides encoding them.

The first Chlamydia trachomatis serovar may be any Chlamydia trachomatis serovar. The second Chlamydia trachomatis serovar may be any Chlamydia trachomatis serovar, excluding that of the first Chlamydia trachomatis serovar.

In one embodiment of the invention the first Chlamydia trachomatis serovar is selected from the list consisting of Chlamydia trachomatis serovars A, B, Ba, C, D, Da, E, F, G, H, I, Ia, J, Ja, K, L1, L2 and L3. In a second embodiment of the invention the first Chlamydia trachomatis serovar is selected from the Chlamydia trachomatis ocular serovars (for example A, B, Ba and C). In another embodiment of the invention the first Chlamydia trachomatis serovar is selected from the Chlamydia trachomatis oculogenital serovars (for example D, Da, E, F, G, H, I, Ia, J, Ja and K). In a further embodiment of the invention the first Chlamydia trachomatis serovar is selected from the Chlamydia trachomatis LGV serovars (for example L1, L2 and L3).

In one embodiment of the invention the second Chlamydia trachomatis serovar is selected from the list consisting of Chlamydia trachomatis serovars A, B, Ba, C, D, Da, E, F, G, H, I, Ia, J, Ja, K, L1, L2 and L3. In a second embodiment of the invention the second Chlamydia trachomatis serovar is selected from the Chlamydia trachomatis ocular serovars (for example A, B, Ba and C). In another embodiment of the invention the second Chlamydia trachomatis serovar is selected from the Chlamydia trachomatis oculogenital serovars (for example D, Da, E, F, G, H, I, Ia, J, Ja and K). In a further embodiment of the invention the second Chlamydia trachomatis serovar is selected from the Chlamydia trachomatis LGV serovars (for example L1, L2 and L3).

In order to maximise the breadth of action of the method and use of the present invention, it may be desirable that the first Chlamydia trachomatis serovar is selected such that there is a high level of sequence identity (for example at least 90%, especially 95%, in particular 98%, more particularly 99% sequence identity) with the majority of other Chlamydia trachomatis serovars (for example at least 50%, especially 70%, in particular 80%, more particularly 90% of other Chlamydia trachomatis serovars).

In order to maximise the practical application of the method and use of the present invention, it may be desirable that the first Chlamydia trachomatis serovar is selected such that there is a high level of sequence identity (for example at least 90%, especially 95%, in particular 98%, more particularly 99% sequence identity) with the majority (for example at least 50%, especially 70%, in particular 80%, more particularly 90%) of common Chlamydia trachomatis serovars (such as the common ocular serovars, the common oculogenital serovars, the common LGV serovars, or a combination of any two of these serovar groups, for example, the common ocular and oculogentical serovars). Common Chlamydia trachomatis ocular serovars include A and B. Common Chlamydia trachomatis oculogenital serovars include D, E, F and I (Lan, J et al. J. Clin. Microbiol. 1995 33(12):3194-3197; Singh, V et al. J. Clin. Microbiol. 2003 41 (6):2700-2702). Common Chlamydia trachomatis LGV serovars include L2.

In one embodiment of the present invention the first Chlamydia trachomatis serovar is Chlamydia trachomatis serovar E. In a second embodiment of the invention the first Chlamydia trachomatis serovar is Chlamydia trachomatis serovar K.

In one embodiment of the invention the second Chlamydia trachomatis serovar is selected from Chlamydia trachomatis serovars D, J and K (for example Chlamydia trachomatis serovar K or J).

In another embodiment of the invention the first Chlamydia trachomatis serovar is Chlamydia trachomatis serovar E and the second Chlamydia trachomatis serovar is selected from Chlamydia trachomatis serovars D, J and K (for example Chlamydia trachomatis serovar K or J).

In one example of the present invention, where the vaccine comprises Ct-089, an immunogenic fragment thereof or polynucleotide encoding it, derived from Chlamydia trachomatis serovar E, the vaccine may be used in the treatment or prophylaxis of infections arising from Chlamydia trachomatis serovars A, B, D, G, H, I, J, K or L2; in particular A, B, D, G, H, I or K; especially A or B.

In a second example of the present invention, where the vaccine comprises Ct-858, an immunogenic fragment thereof or polynucleotide encoding it, derived from Chlamydia trachomatis serovar E, the vaccine may be used in the treatment or prophylaxis of infections arising from Chlamydia trachomatis serovars A, B, D, G, H, I, J, K or L2; in particular J or L2.

In a further example of the present invention, where the vaccine comprises Ct-875, an immunogenic fragment thereof or polynucleotide encoding it, derived from Chlamydia trachomatis serovar E, the vaccine may be used in the treatment or prophylaxis of infections arising from Chlamydia trachomatis serovars A, B, D, G, H, I, J, K or L2; in particular A, B, D, G, H, I or K.

The first and second Chlamydia trachomatis serovars may be associated with the same disease state (for example they may both be ocular serovars or both be oculogenital serovars), or the first and second Chlamydia trachomatis serovars may be associated with different disease states (for example the first Chlamydia trachomatis serovar may an oculogenital serovar and the second Chlamydia trachomatis serovar may be an ocular serovar, or vice versa).

In the event that the vaccine of use in the present invention comprises more than one protein, immunogenic fragment thereof or polynucleotide encoding them, selected from the list consisting of Ct-089, Ct-858 and Ct-875, it should be noted that each protein, immunogenic fragment thereof or polynucleotide encoding them, may optionally be derived from a different first Chlamydia trachomatis serovar which may be independently selected.

Cross-protection vaccines of use in the present invention may also comprise additional Chlamydia antigens (i.e. antigens other than Ct-089, Ct-858 and Ct-875 proteins, immunogenic fragments thereof or polynucleotides encoding them), for example 1, 2, 3, 4 or 5 other antigens (selected for example from Momp, Ct-622, PmpGpd and PmpDpd). Additional antigens in cross-protection vaccines may also include Ct-089, Ct-858 and Ct-875 proteins, immunogenic fragments thereof or polynucleotides encoding them which are derived from the second serovar.

In a further embodiment of the invention Chlamydia polypeptides and polynucleotides that may be used in accordance with the invention include those from serovars associated with trachoma such as serovars A, B, Ba and C.

Thus the compositions according to the invention may employ the polypeptide sequences given above or immunogenic fragments of these, or polynucleotide sequences encoding these which may be for example the polynucleotide sequences given above or fragments of these encoding immunogenic fragments of the polypeptides.

In particular embodiments:

(i) the Ct-089 and Ct-858 components of the composition according to the invention may be a polypeptide having at least 95% homology to the polypeptide of SEQ ID NO: 16 (C. trachomatis serovar E) or an immunogenic fragment thereof, or a polypeptide having at least 95% homology to the polypeptide of SEQ ID NO: 6 (C. trachomatis serovar E) or an immunogenic fragment thereof, respectively, or polynucleotides encoding these. Alternatively the Ct-089 and Ct-858 components of the composition may show at least 95% homology to any one of the Ct-089 and Ct-858 polypeptide and polynucleotide sequences from other C. trachomatis serovars which are described herein.

(ii) A Ct-875 component may be a polypeptide having at least 95% homology to the polypeptide of SEQ ID NO: 8 (C. trachomatis serovar E) or an immunogenic fragment thereof, or polynucleotides encoding these.

Alternatively the Ct-875 component of the composition may show at least 95% homology to any one of the Ct-875 polypeptide and polynucleotide sequences from other C. trachomatis serovars which are described herein.

(iii) A PmpDpd component may be a polypeptide having at least 95% homology to the polypeptide of SEQ ID NO: 14 (C. trachomatis serovar LII) or an immunogenic fragment thereof, or polynucleotides encoding these.

(iv) A PmpGpd component may be a polypeptide having at least 95% homology to the polypeptide of SEQ ID NO: 12 (C. trachomatis serovar LII) or an immunogenic fragment thereof, or polynucleotides encoding these.

(v) A Momp component may be a polypeptide having at least 95% homology to the polypeptide of SEQ ID NO: 4 (C. trachomatis serovar F) or an immunogenic fragment thereof, or polynucleotides encoding these.

(vi) A Swib component may be a polypeptide having at least 95% homology to the polypeptide of SEQ ID NO: 8 (C. trachomatis serovar LII) or an immunogenic fragment thereof, or polynucleotides encoding these.

The antigens described herein include polymorphic variants and conservatively modified variations, as well as inter-strain and interspecies Chlamydia homologues. In addition, the antigens described herein include subsequences or truncated sequences.

The antigens described herein may be in the form of fusion proteins. The fusion proteins may also contain additional polypeptides, optionally heterologous peptides from Chlamydia or other sources. These antigens may be modified, for example, by adding linker peptide sequences as described below. These linker peptides may be inserted between one or more polypeptides which make up each of the fusion proteins.

The antigens described herein may also be in the form of chemical conjugates.

The invention further relates to immunogenic compositions and vaccine compositions comprising the compositions of Chlamydia antigens according to the invention, together with a pharmaceutically acceptable carrier and optionally an immunostimulant. The compositions of the present invention may further comprise other components designed to enhance the antigenicity of the antigens or to improve these antigens in other aspects, for example, the isolation of these antigens through addition of a stretch of histidine residues at one end of the antigen. The addition of a stretch of histidine residues at one end of the antigen may also improve expression. The compositions of the invention can comprise additional copies of antigens, or additional polypeptides or polynucleotides from Chlamydia sp. The compositions of the invention can also comprise additional heterologous polypeptides or polynucleotides from other non-Chlamydia sources. For example, the compositions of the invention can include polypeptides or nucleic acids encoding polypeptides, wherein the polypeptide enhances expression of the antigen, e.g., NS1, an influenza virus protein, or an immunogenic portion thereof (see, e.g. WO99/40188 and WO93/04175). The nucleic acids of the invention can be engineered based on codon preference in a species of choice, e.g., humans.

The compositions of the invention may further comprise adjuvants, e.g., MPL, 3D-MPL, IFA, ENHANZYN (Detox), QS21, CWS, TDM, AGP, CPG, Leif, saponin, and saponin mimetics, and derivatives thereof. Alternatively or in addition, the compositions of the invention can comprise BCG or Pvac as an adjuvant.

### DEFINITIONS

“Fusion polypeptide” or “fusion protein” refers to a protein having at least two Chlamydia polypeptides (which may be the same, or may be different) covalently linked, either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order. This term also refers to conservatively modified variants, polymorphic variants, alleles, mutants, subsequences, interspecies homologs, and immunogenic fragments of the antigens that make up the fusion protein. Fusion proteins of the invention can also comprise additional copies of a component antigen or immunogenic fragment thereof.

A polynucleotide sequence encoding a fusion protein of the invention hybridizes under stringent conditions to at least two nucleotide sequences, each encoding an antigen polypeptide selected from the group consisting of Ct-681 (Momp) or an immunogenic fragment thereof, Ct-871 (PmpG) or an immunogenic fragment thereof, Ct-812 (PmpD) or an immunogenic fragment thereof, Ct-089 or an immunogenic fragment thereof, Ct-858 or an immunogenic fragment thereof, Ct-875 or an immunogenic fragment thereof, Ct-460 (swib) or an immunogenic fragment thereof, and Ct-622 or an immunogenic fragment thereof. The polynucleotide sequences encoding the individual antigens of the fusion polypeptide therefore include conservatively modified variants, polymorphic variants, alleles, mutants, subsequences, immunogenic fragments, and interspecies homologs of Ct-681 (Momp), Ct-871 (PmpG), Ct-812 (PmpD), Ct-089, Ct-858, Ct-875, Ct-460 (swib), and Ct-622. The polynucleotide sequences encoding the individual polypeptides of the fusion protein can be in any order.

In some embodiments, the individual polypeptides of the fusion protein are in order (N- to C-terminus) from large to small. Large antigens are approximately 30 to 150 kD in size, medium antigens are approximately 10 to 30 kD in size, and small antigens are approximately less than 10 kD in size. The sequence encoding the individual polypeptide may be as small as, e.g., an immunogenic fragment such as an individual CTL epitope encoding about 8 to 9 amino acids, or, e.g., an HTL or B cell epitope. The fragment may also include multiple epitopes.

A fusion polypeptide of the invention specifically binds to antibodies raised against at least two antigen polypeptides selected from Ct-681 (Momp) or an immunogenic fragment thereof, Ct-871 (PmpG) or an immunogenic fragment thereof, Ct-812 (PmpD) or an immunogenic fragment thereof, Ct-089 or an immunogenic fragment thereof, Ct-858 or an immunogenic fragment thereof, Ct-875 or an immunogenic fragment thereof, Ct-460 (swib) or an immunogenic fragment thereof, and Ct-622 or an immunogenic fragment thereof. The antibodies can be polyclonal or monoclonal. Optionally, the fusion polypeptide specifically binds to antibodies raised against the fusion junction of the antigens, which antibodies do not bind to the antigens individually, i.e., when they are not part of a fusion protein. The fusion polypeptides optionally comprise additional polypeptides, e.g., three, four, five, six, or more polypeptides, up to about 25 polypeptides, optionally heterologous polypeptides or repeated homologous polypeptides, fused to the at least two antigens. The additional polypeptides of the fusion protein are optionally derived from Chlamydia as well as other sources, such as other bacterial, viral, or invertebrate, vertebrate, or mammalian sources. The individual polypeptides of the fusion protein can be in any order. As described herein, the fusion protein can also be linked to other molecules, including additional polypeptides. The compositions of the invention can also comprise additional polypeptides that are unlinked to the fusion proteins of the invention. These additional polypeptides may be heterologous or homologous polypeptides.

The term “fused” refers to the covalent linkage between two polypeptides in a fusion protein. The polypeptides are typically joined via a peptide bond, either directly to each other or via an amino acid linker. Optionally, the peptides can be joined via non-peptide covalent linkages known to those of skill in the art.

“FL” refers to full-length, i.e., a polypeptide that is the same length as the wild-type polypeptide.

The term “immunogenic fragment thereof” refers to a polypeptide comprising an epitope that is recognized by cytotoxic T lymphocytes, helper T lymphocytes or B cells. Methods of determining epitope regions of a sequence are described elsewhere herein. Suitably, the immunogenic fragment will comprise at least 30%, suitably at least 50%, especially at least 75% and in particular at least 90% (e.g. 95% or 98%) of the amino acids in the reference sequence. The immunogenic fragment will suitably comprise all of the epitope regions of the reference sequence.

An adjuvant refers to the components in a vaccine or therapeutic composition that increase the specific immune response to the antigen (see, e.g., Edelman, AIDS Res. Hum Retroviruses 8:1409-1411 (1992)). Adjuvants induce immune responses of the Th1-type and Th-2 type response. Th1-type cytokines (e.g., IFN-γ, IL-2, and IL-12) tend to favor the induction of cell-mediated immune response to an administered antigen, while Th-2 type cytokines (e.g., IL-4, IL-5, Il-6, IL-10 and TNF-β) tend to favor the induction of humoral immune responses. Any of a variety of adjuvants may be employed in the vaccines of this invention to enhance the immune response. Some adjuvants contain a substance designed to protect the antigen from rapid catabolism, such as aluminum hydroxide or mineral oil, and a specific or nonspecific stimulator of immune responses, such as lipid A, Bortadella pertussis or Mycobacterium tuberculosis. Suitable adjuvants are commercially available and include, for example, Freund's Incomplete Adjuvant and Freund's Complete Adjuvant (Difco Laboratories) and Merck Adjuvant 65 (Merck and Company, Inc., Rahway, N.J.). Other suitable adjuvants include alum, biodegradable microspheres, monophosphoryl lipid A, quil A, SBAS1c, SBAS2 (Ling et al., 1997, Vaccine 15:1562-1567), SBAS7, Al(OH)₃ and CpG oligonucleotide (WO96/02555). Suitable adjuvants for use in the invention are discussed in more detail below.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

A polynucleotide of the invention may contain a number of silent variations (for example, 1-5, in particular 1 or 2, and especially 1 codon(s) may be altered) when compared to the reference sequence. A polynucleotide of the invention may contain a number of non-silent conservative variations (for example, 1-5, in particular 1 or 2, and especially 1 codon(s) may be altered) when compared to the reference sequence. Those skilled in the art will recognise that a particular polynucleotide sequence may contain both silent and non-silent conservative variations.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

A polypeptide of the invention may contain a number of conservative variations (for example, 1-5, in particular 1 or 2, and especially 1 amino acid residue(s) may be altered) when compared to the reference sequence. In general, such conservative substitutions will fall within one of the amino-acid groupings specified below, though in some circumstances other substitutions may be possible without substantially affecting the immunogenic properties of the antigen. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

Suitably amino-acid substitutions are restricted to non-epitope regions of an antigen.

Polypeptide sequence variants may also include those wherein additional amino acids are inserted compared to the reference sequence, for example, such insertions may occur at 1 or 2 locations (suitably 1) and may involve the addition of 50 or fewer amino acids (such as 20 or fewer, in particular 10 or fewer, especially 5 or fewer) at each location. Suitably such insertions so not occur in the region of an epitope, and do not therefore have a significant impact on the immunogenic properties of the antigen. One example of insertions includes a short stretch of histidine residues (e.g. 1-6 residues) to aid expression and/or purification of the antigen in question.

Other polypeptide sequence variants include those wherein amino acids have been deleted compared to the reference sequence, for example, such deletions may occur at 1 or 2 locations (suitably 1) and may, for example, involve the deletion of 50 or fewer amino acids (such as 20 or fewer, in particular 10 or fewer, especially 5 or fewer) at each location. Suitably such insertions so not occur in the region of an epitope, and do not therefore have a significant impact on the immunogenic properties of the antigen.

Methods of determining the epitope regions of an antigen are described and exemplified elsewhere herein.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554. (1990); Marks et al., Biotechnology 10:779-783 (1992)).

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to fusion proteins can be selected to obtain only those polyclonal antibodies that are specifically immunoreactive with fusion protein and not with individual components of the fusion proteins. This selection may be achieved by subtracting out antibodies that cross-react with the individual antigens. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

Polynucleotides may comprise a native sequence (i.e., an endogenous sequence that encodes an individual antigen or a portion thereof) or may comprise a variant of such a sequence. Polynucleotide variants may contain one or more substitutions, additions, deletions and/or insertions such that the biological activity of the encoded fusion polypeptide is not diminished, relative to a fusion polypeptide comprising native antigens. Variants preferably exhibit at least about 70% identity, more preferably at least about 80% identity and most preferably at least about 90% identity to a polynucleotide sequence that encodes a native polypeptide or a portion thereof.

The terms “identical” or percent “identity,” in the context of two or more nucleic acids or polypeptide sequences, refer to two or more sequences or subsequences that are the same or have a specified percentage of amino acid residues or nucleotides that are the same (i.e., 70% identity, optionally 75%, 80%, 85%, 90%, or 95% (e.g. 98%) identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Optionally, the identity exists over a region that is at least about 25 to about 50 amino acids or nucleotides in length, or optionally over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 25 to 500, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted by, for example, the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (hftp://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

### Polynucleotide Compositions

As used herein, the terms “DNA segment” and “polynucleotide” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

As will be understood by those skilled in the art, the DNA segments of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

The terms “isolated,” “purified,” or “biologically pure” therefore refer to material that is substantially or essentially free from components that normally accompany it as found in its native state. Of course, this refers to the DNA segment as originally isolated, and does not exclude other isolated proteins, genes, or coding regions later added to the composition by the hand of man. Purity and homogeneity are typically determined using analytical chemistry techniques such as polyacrylamide gel electrophoresis or high performance liquid chromatography. A protein that is the predominant species present in a preparation is substantially purified. An isolated nucleic acid is separated from other open reading frames that flank the gene and encode proteins other than the gene.

As will be recognized by the skilled artisan, polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a Chlamydia antigen or a portion thereof) or may comprise a variant, or a biological or antigenic functional equivalent of such a sequence. Polynucleotide variants may contain one or more substitutions, additions, deletions and/or insertions, as further described below, preferably such that the immunogenicity of the encoded polypeptide is not diminished, relative to a native tumor protein. The effect on the immunogenicity of the encoded polypeptide may generally be assessed as described herein. The term “variants” also encompasses homologous genes of xenogenic origin.

In additional embodiments, the present invention provides isolated polynucleotides and polypeptides comprising various lengths of contiguous stretches of sequence identical to or complementary to one or more of the sequences disclosed herein. For example, polynucleotides are provided by this invention that comprise at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative DNA segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention, for example polynucleotides that are optimized for human and/or primate codon selection. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

### Polynucleotide Identification and Characterization

Polynucleotides may be identified, prepared and/or manipulated using any of a variety of well-established techniques. For example, a polynucleotide may be identified, as described in more detail below, by screening a microarray of cDNAs. Such screens may be performed, for example, using a Synteni microarray (Palo Alto, Calif.) according to the manufacturer's instructions (and essentially as described by Schena et al., Proc. Natl. Acad. Sci. USA 93:10614-10619 (1996) and Heller et al., Proc. Natl. Acad. Sci. USA 94:2150-2155 (1997)). Alternatively, polynucleotides may be amplified from cDNA prepared from cells expressing the proteins described herein, such as C. trachomatis cells. Such polynucleotides may be amplified via polymerase chain reaction (PCR). For this approach, sequence-specific primers may be designed based on the sequences provided herein, and may be purchased or synthesized.

An amplified portion of a polynucleotide of the present invention may be used to isolate a full-length gene from a suitable library (e.g., a C. trachomatis cDNA library) using well-known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²P) using well-known techniques. A bacterial or bacteriophage library is then generally screened by hybridizing filters containing denatured bacterial colonies (or lawns containing phage plaques) with the labeled probe (see Sambrook et al., Molecular Cloning: A Laboratory Manual (1989)). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full-length cDNA molecule can be generated by ligating suitable fragments, using well-known techniques.

Alternatively, there are numerous amplification techniques for obtaining a full-length coding sequence from a partial cDNA sequence. Within such techniques, amplification is generally performed via PCR. Any of a variety of commercially available kits may be used to perform the amplification step. Primers may be designed using, for example, software well known in the art. Primers are preferably 22-30 nucleotides in length have a GC content of at least 50% and anneal to the target sequence at temperatures of about 68° C. to 72° C. The amplified region may be sequenced as described above, and overlapping sequences assembled into a contiguous sequence.

One such amplification technique is inverse PCR (see Triglia et al., Nucl. Acids Res. 16:8186 (1988)), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19 (1991)) and walking PCR (Parker et al., Nucl. Acids. Res. 19:3055-60 (1991)). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

### Polynucleotide Expression in Host Cells

In other embodiments of the invention, polynucleotide sequences or fragments thereof which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life that is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences may be ligated to a heterologous sequence to encode a fusion protein. For example, to screen peptide libraries for inhibitors of polypeptide activity, it may be useful to encode a chimeric protein that can be recognized by a commercially available antibody. A fusion protein may also be engineered to contain a cleavage site located between the polypeptide-encoding sequence and the heterologous protein sequence, so that the polypeptide may be cleaved and purified away from the heterologous moiety.

Sequences encoding a desired polypeptide may be synthesized, in whole or in part, using chemical methods well known in the art (see Caruthers, M. H. et al., Nucl. Acids Res. Symp. Ser. pp. 215-223 (1980), Horn et al., Nucl. Acids Res. Symp. Ser. pp. 225-232 (1980)). Alternatively, the protein itself may be produced using chemical methods to synthesize the amino acid sequence of a polypeptide, or a portion thereof. For example, peptide synthesis can be performed using various solid-phase techniques (Roberge et al., Science 269:202-204 (1995)) and automated synthesis may be achieved, for example, using the ABI 431A Peptide Synthesizer (Perkin Elmer, Palo Alto, Calif.).

A newly synthesized peptide may be substantially purified by preparative high performance liquid chromatography (e.g., Creighton, Proteins, Structures and Molecular Principles (1983)) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

In order to express a desired polypeptide, the nucleotide sequences encoding the polypeptide, or functional equivalents, may be inserted into appropriate expression vector, i.e., a vector that contains the necessary elements for the transcription and translation of the inserted coding sequence. Methods that are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke &Schuster, J. Biol. Chem. 264:5503-5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. Other vectors containing constitutive or inducible promoters include GAP, PGK, GAL and ADH. For reviews, see Ausubel et al. (supra), Grant et al., Methods Enzymol. 153:516-544 (1987) and Romas et al. Yeast 8 423-88 (1992).

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill Yearbook of Science and Technology pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard et al., Proc. Natl. Acad. Sci. U.S.A. 91:3224-3227 (1994)).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus that is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers that are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines that stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler et al., Cell 11:223-32 (1977)) and adenine phosphoribosyltransferase (Lowy et al., Cell 22:817-23 (1990)) genes which can be employed in tk.sup.- or aprt.sup.-cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisd, which allows cells to utilize histinol in place of histidine (Hartman & Mulligan, Proc. Natl. Acad. Sci. U.S.A. 85:8047-51 (1988)). Recently, the use of visible markers has gained popularity with such markers as anthocyanins, β-glucuronidase and its substrate GUS, and luciferase and its substrate luciferin, being widely used not only to identify transformants, but also to quantify the amount of transient or stable protein expression attributable to a specific vector system (Rhodes et al., Methods Mol. Biol. 55:121-131 (1995)).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques that include membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences that direct secretion of the encoded polypeptide through a prokaryotic or eukaryotic cell membrane. Other recombinant constructions may be used to join sequences encoding a polypeptide of interest to nucleotide sequence encoding a polypeptide domain that will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath et al., Prot. Exp. Purif. 3:263-281 (1992) while the enterokinase cleavage site provides a means for purifying the desired polypeptide from the fusion protein. A discussion of vectors which contain fusion proteins is provided in Kroll et al., DNA Cell Biol. 12:441-453 (1993)).

In addition to recombinant production methods, polypeptides of the invention, and fragments thereof, may be produced by direct peptide synthesis using solid-phase techniques (Merrifield, J. Am. Chem. Soc. 85:2149-2154 (1963)). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

### In Vivo Polynucleotide Delivery Techniques

In additional embodiments, genetic constructs comprising the compositions of polynucleotides of the invention are introduced into cells in vivo. This may be achieved using any of a variety or well-known approaches, several of which are outlined below for the purpose of illustration.

1. Adenovirus

One of the preferred methods for in vivo delivery of one or more nucleic acid sequences involves the use of an adenovirus expression vector. “Adenovirus expression vector” is meant to include those constructs containing adenovirus sequences sufficient to (a) support packaging of the construct and (b) to express a polynucleotide that has been cloned therein in a sense or antisense orientation. Of course, in the context of an antisense construct, expression does not require that the gene product be synthesized.

The expression vector comprises a genetically engineered form of an adenovirus. Knowledge of the genetic organization of adenovirus, a 36 kb, linear, double-stranded DNA virus, allows substitution of large pieces of adenoviral DNA with foreign sequences up to 7 kb (Grunhaus & Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5□-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). In nature, adenovirus can package approximately 105% of the wild-type genome (Ghosh-Choudhury et al., 1987), providing capacity for about 2 extra kB of DNA. Combined with the approximately 5.5 kB of DNA that is replaceable in the E1 and E3 regions, the maximum capacity of the current adenovirus vector is under 7.5 kB, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the currently preferred helper cell line is 293.

Recently, Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain a conditional replication-defective adenovirus vector for use in the present invention, since Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus & Horwitz, 1992; Graham & Prevec, 1992). Recently, animal studies suggested that recombinant adenovirus could be used for gene therapy (Stratford-Perricaudet & Perricaudet, 1991; Strafford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993).

2. Retroviruses

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding one or more oligonucleotide or polynucleotide sequences of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas & Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

3. Adeno-Associated Viruses

AAV (Ridgeway, 1988; Hermonat & Muzycska, 1984) is a parovirus, discovered as a contamination of adenoviral stocks. It is a ubiquitous virus (antibodies are present in 85% of the US human population) that has not been linked to any disease. It is also classified as a dependovirus, because its replications is dependent on the presence of a helper virus, such as adenovirus. Five serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter (Muzyczka & McLaughlin, 1988).

The AAV DNA is approximately 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three viral promoters have been identified and named p5, p19, and p40, according to their map position. Transcription from p5 and p19 results in production of rep proteins, and transcription from p40 produces the capsid proteins (Hermonat & Muzyczka, 1984).

There are several factors that prompted researchers to study the possibility of using rAAV as an expression vector. One is that the requirements for delivering a gene to integrate into the host chromosome are surprisingly few. It is necessary to have the 145-bp ITRs, which are only 6% of the AAV genome. This leaves room in the vector to assemble a 4.5-kb DNA insertion. While this carrying capacity may prevent the AAV from delivering large genes, it is amply suited for delivering the antisense constructs of the present invention.

AAV is also a good choice of delivery vehicles due to its safety. There is a relatively complicated rescue mechanism: not only wild type adenovirus but also AAV genes are required to mobilize rAAV. Likewise, AAV is not pathogenic and not associated with any disease. The removal of viral coding sequences minimizes immune reactions to viral gene expression, and therefore, rAAV does not evoke an inflammatory response.

4. Other Viral Vectors as Expression Constructs

Other viral vectors may be employed as expression constructs in the present invention for the delivery of oligonucleotide or polynucleotide sequences to a host cell. Vectors derived from viruses such as vaccinia virus (Ridgeway, 1988; Coupar et al., 1988), lentiviruses, polioviruses and herpes viruses may be employed. They offer several attractive features for various mammalian cells (Friedmann, 1989; Ridgeway, 1988; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. (1991) introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

Additional ‘viral’ vectors include virus like particles (VLPs) and phages.

5. Non-Viral Vectors

In order to effect expression of the oligonucleotide or polynucleotide sequences of the present invention, the expression construct must be delivered into a cell. This delivery may be accomplished in vitro, as in laboratory procedures for transforming cells lines, or in vivo or ex vivo, as in the treatment of certain disease states. As described above, one preferred mechanism for delivery is via viral infection where the expression construct is encapsulated in an infectious viral particle.

Once the expression construct has been delivered into the cell the nucleic acid encoding the desired oligonucleotide or polynucleotide sequences may be positioned and expressed at different sites. In certain embodiments, the nucleic acid encoding the construct may be stably integrated into the genome of the cell. This integration may be in the specific location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In certain embodiments of the invention, the expression construct comprising one or more oligonucleotide or polynucleotide sequences may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty & Reshef (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

### Polypeptide Compositions

The present invention provides polypeptide compositions as described herein. Generally, a polypeptide composition of the invention will be a combination of isolated polypeptides or immunogenic fragments thereof. Alternatively, some or all of the polypeptide antigens in an inventive composition may be within a fusion protein. For example, in an inventive composition comprising three antigens: (i) the antigens may be provided in the form of three isolated polypeptides (ii) all three polypeptides antigens may be provided in a single fusion protein (iii) two of the antigens may be provided in a fusion protein, with the third provided in isolated form. The polypeptides of the combination may be encoded by a polynucleotide sequence or sequences disclosed herein or a sequence or sequences that hybridize under moderately stringent conditions to a polynucleotide sequence or sequences disclosed herein. Alternatively, the polypeptides may be defined as polypeptides each comprising a contiguous amino acid sequence from an amino acid sequence disclosed herein, or which polypeptides each comprise an entire amino acid sequence disclosed herein.

Immunogenic portions may generally be identified using well-known techniques, such as those summarized in Paul, Fundamental Immunology, 3rd ed., 243-247 (1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques. An immunogenic portion of a Chlamydia sp. protein is a portion that reacts with such antisera and/or T-cells at a level that is not substantially less than the reactivity of the full-length polypeptide (e.g., in an ELISA and/or T-cell reactivity assay). Such immunogenic portions may react within such assays at a level that is similar to or greater than the reactivity of the full-length polypeptide. Such screens may generally be performed using methods well known to those of ordinary skill in the art, such as those described in Harlow & Lane, Antibodies: A Laboratory Manual (1988). For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

Polypeptides may be prepared using any of a variety of well-known techniques. Recombinant polypeptides encoded by DNA sequences as described above may be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast, and higher eukaryotic cells, such as mammalian cells and plant cells. Preferably, the host cells employed are E. coli, yeast or a mammalian cell line such as COS or CHO. Supernatants from suitable host/vector systems that secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Polypeptides of the invention, immunogenic fragments thereof which may have for example less than about 100 amino acids, or less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146 (1963). Equipment for automated synthesis of polypeptides is commercially available from suppliers such as Perkin Elmer/Applied BioSystems Division (Foster City, Calif.), and may be operated according to the manufacturer's instructions.

Within certain specific embodiments, a polypeptide may be a fusion protein that comprises multiple polypeptides as described herein, or that comprises at least one polypeptide as described herein and an unrelated sequence, such as a known protein. Such a fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Thus, a fusion protein may be expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides. Typically fusion proteins comprising two or more antigens may omit the initiation codon (Met) from the second and subsequent antigens.

A peptide linker sequence may be employed to separate the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46 (1985); Murphy et al., Proc. Natl. Acad. Sci. USA 83:8258-8262 (1986); U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

Thus the compositions according to the invention may comprise one or more fusion proteins. Such proteins comprise a polypeptide component of the composition as described herein together with an unrelated immunogenic protein. The immunogenic protein may for example be capable of eliciting a recall response. Examples of such proteins include tetanus, tuberculosis and hepatitis proteins (see, e.g., Stoute et al., New Engl. J. Med. 336:86-91 (1997)).

Within certain embodiments, an immunological fusion partner is derived from protein D, a surface protein of the gram-negative bacterium Haemophilus influenza B (WO 91/18926). A protein D derivative may comprise approximately the first third of the protein (e.g., the first N-terminal 100-110 amino acids), and a protein D derivative may be lipidated. Within certain embodiments, the first 109 residues of a lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292 (1986)). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798 (1992)). Within a preferred embodiment, a repeat portion of LYTA may be incorporated into a fusion protein. A repeat portion is found in the C-terminal region starting at residue 178. A particularly preferred repeat portion incorporates residues 188-305.

In general, polypeptides (including fusion proteins) and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

### T Cells

Immunotherapeutic compositions may also, or alternatively, comprise T cells specific for a Chlamydia antigen. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also U.S. Pat. No. 5,240,856; U.S. Pat. No. 5,215,926; WO 89/06280; WO 91/16116 and WO 92/07243). Alternatively, T cells may be derived from related or unrelated humans, non-human mammals, cell lines or cultures.

T cells may be stimulated with a polypeptide of the invention, polynucleotide encoding such a polypeptide, and/or an antigen presenting cell (APC) that expresses such a polypeptide. Such stimulation is performed under conditions and for a time sufficient to permit the generation of T cells that are specific for the polypeptide. Preferably, the polypeptide or polynucleotide is present within a delivery vehicle, such as a microsphere, to facilitate the generation of specific T cells.

T cells are considered to be specific for a polypeptide of the invention if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070 (1994)). Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a polypeptide of the invention (100 ng/ml-100 μg/ml, preferably 200 ng/ml-25 μg/ml) for 3-7 days should result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1 (1998)). T cells that have been activated in response to a polypeptide, polynucleotide or polypeptide-expressing APC may be CD4⁺ and/or CD8⁺. Protein-specific T cells may be expanded using standard techniques. Within preferred embodiments, the T cells are derived from a patient, a related donor or an unrelated donor, and are administered to the patient following stimulation and expansion.

For therapeutic purposes, CD4⁺ or CD8⁺ T cells that proliferate in response to a polypeptide, polynucleotide or APC can be expanded in number either in vitro or in vivo. Proliferation of such T cells in vitro may be accomplished in a variety of ways. For example, the T cells can be re-exposed to a polypeptide, or a short peptide corresponding to an immunogenic portion of such a polypeptide, with or without the addition of T cell growth factors, such as interleukin-2, and/or stimulator cells that synthesize the polypeptide. Alternatively, one or more T cells that proliferate in the presence of the protein can be expanded in number by cloning. Methods for cloning cells are well known in the art, and include limiting dilution.

### Diagnostic Methods

Prior infection of an individual by Chlamydia will often be detectable by ELISA. Individuals carrying Chlamydia specific antibodies (‘seropositive’) having been infected previously. However, it is not uncommon for individuals who have been infected by Chlamydia previously to be found to be seronegative upon testing, i.e. no Chlamydia specific antibodies may be detected. As a result of the prior infection, despite testing seronegative, such individuals respond strongly to restimulation by Chlamydial antigens (relative to seronegative individuals which have not previously been infected), in particular to the various Chlamydial antigen combinations which have been described previously herein.

Therefore, in a further aspect of the present invention there is provided a method for determining prior Chlamydial infection in an individual comprising:
(i) obtaining a sample from the individual;
(ii) contacting said sample with a combination of two or more Chlamydia proteins or immunogenic fragments thereof or a polynucleotide or polynucleotides encoding them, said two or more proteins or immunogenic fragments selected from Swib, Momp, Ct-858, Ct-875, Ct-622, Ct-089, passenger domain of PmpG (PmpGpd) and passenger domain of PmpD (PmpDpd);
(iii) quantifying the sample response.

The sample may for example be whole blood or purified cells. Suitably the sample will contain peripheral blood mononucleated cells (PBMC). In one embodiment of the invention the individual will be seropositive. In a second embodiment of the invention the individual will be seronegative.

The sample response may be quantified by a range of means known to those skilled in the art, including the monitoring of lymphocyte proliferation or the production of specific cytokines or antibodies in the presence of the combination of Chlamydial antigens. For example, T-cell ELISPOT may be used to monitor cytokines such as interferon gamma (IFNγ), interleukin 2 (IL2) and interleukin 5 (IL5). B-cell ELLISPOT may be used to monitor the stimulation of Chlamydia specific antigens.

Methods of quantifying sample response are illustrated in the Examples herein (specifically Example 9). When using such method, a positive response to an antigen may be defined by a signal to noise ratio (S/N ratio) of at least 2:1 (for example, at least 3:1).

In a further aspect of the present invention methods are provided for using one or more of the antigen combinations (or immunogenic fragments thereof or nucleotides encoding them) described above to diagnose prior Chlamydial infection using a skin test. As used herein, a “skin test” is any assay performed directly on a patient in which a delayed-type hypersensitivity (DTH) reaction (such as swelling, reddening or dermatitis) is measured following intradermal injection of an antigen combination (or immunogenic fragments thereof or nucleotides encoding them) as described above. Such injection may be achieved using any suitable device sufficient to contact the antigen combinations with dermal cells of the patient, such as a tuberculin syringe or 1 mL syringe. The reaction is measured after a period of time, for example at least 48 hours after injection, especially 48-72 hours.

The DTH reaction is a cell-mediated immune response, which is greater in patients that have been exposed previously to the test antigen. The response may be measured visually, using a ruler. In general, a response that is greater than about 0.5 cm in diameter, especially greater than about 1.0 cm in diameter, is a positive response, indicative of prior Chlamydial infection, which may or may not be manifested as an active disease.

For use in a skin test, the combinations of this invention are suitably formulated as pharmaceutical compositions containing a physiologically acceptable carrier. Suitably, the carrier employed in such pharmaceutical compositions is a saline solution with appropriate preservatives, such as phenol and/or Tween 80™.

### Pharmaceutical Compositions

In additional embodiments, the present invention concerns formulation of the polynucleotide, polypeptide, T-cell and/or antibody compositions disclosed herein in pharmaceutically-acceptable or physiologically-acceptable solutions for administration to a cell or an animal, either alone, or in combination with one or more other modalities of therapy. Such compositions are also useful for diagnostic uses.

It will also be understood that, if desired, the nucleic acid segments, RNA, DNA or PNA compositions that express a composition of polypeptides as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation. Other routes of administration include via the mucosal surfaces, for example intravaginal administration.

1. Oral Delivery

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. No. 5,641,515; U.S. Pat. No. 5,580,579 and U.S. Pat. No. 5,792,451, each specifically incorporated herein by reference in its entirety). The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Typically, these formulations may contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

2. Injectable Delivery

In certain circumstances it will be desirable to deliver the pharmaceutical compositions disclosed herein parenterally, intravenously, intramuscularly, or even intraperitoneally as described in U.S. Pat. No. 5,543,158; U.S. Pat. No. 5,641,515 and U.S. Pat. No. 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

3. Mucosal Delivery

(i) Nasal Delivery

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

(ii) Intravaginal Delivery

In other embodiments of the invention the pharmaceutical compositions may be formulated for intravaginal delivery. Such formulations may be prepared as liquids, semi-solids or solids (including for example, creams, ointments, gels etc), or may be contained within a physical delivery system such as a pessary, sponge, vaginal ring or film.

(iii) Ocular Delivery

In further embodiments of the invention the pharmaceutical compositions may be formulated for ocular delivery. Such formulations will desirably be clear and colorless.

5. Liposome-, Nanocapsule-, and Microparticle-Mediated Delivery

In certain embodiments, the inventors contemplate the use of liposomes, nanocapsules, microparticles, microspheres, lipid particles, vesicles, and the like, for the introduction of the compositions of the present invention into suitable host cells. In particular, the compositions of the present invention may be formulated for delivery either encapsulated in a lipid particle, a liposome, a vesicle, a nanosphere, or a nanoparticle or the like.

Such formulations may be preferred for the introduction of pharmaceutically-acceptable formulations of the nucleic acids or constructs disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally resistant to transfection by other procedures including T cell suspensions, primary hepatocyte cultures and PC 12 cells (Renneisen et al., 1990; Muller et al., 1990). In addition, liposomes are free of the DNA length constraints that are typical of viral-based delivery systems. Liposomes have been used effectively to introduce genes, drugs (Heath & Martin, 1986; Heath et al., 1986; Balazsovits et al., 1989; Fresta & Puglisi, 1996), radiotherapeutic agents (Pikul et al., 1987), enzymes (Imaizumi et al., 1990a; Imaizumi et al., 1990b), viruses (Faller & Baltimore, 1984), transcription factors and allosteric effectors (Nicolau & Gersonde, 1979) into a variety of cultured cell lines and animals. In addition, several successful clinical trails examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori & Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977; 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

In addition to temperature, exposure to proteins can alter the permeability of liposomes. Certain soluble proteins, such as cytochrome c, bind, deform and penetrate the bilayer, thereby causing changes in permeability. Cholesterol inhibits this penetration of proteins, apparently by packing the phospholipids more tightly. It is contemplated that the most useful liposome formations for antibiotic and inhibitor delivery will contain cholesterol.

The ability to trap solutes varies between different types of liposomes. For example, MLVs are moderately efficient at trapping solutes, but SUVs are extremely inefficient. SUVs offer the advantage of homogeneity and reproducibility in size distribution, however, and a compromise between size and trapping efficiency is offered by large unilamellar vesicles (LUVs). These are prepared by ether evaporation and are three to four times more efficient at solute entrapment than MLVs.

In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature.

Liposomes interact with cells via four different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

The fate and disposition of intravenously injected liposomes depend on their physical properties, such as size, fluidity, and surface charge. They may persist in tissues for h or days, depending on their composition, and half lives in the blood range from min to several h. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominate site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

Targeting is generally not a limitation in terms of the present invention. However, should specific targeting be desired, methods are available for this to be accomplished. Antibodies may be used to bind to the liposome surface and to direct the antibody and its drug contents to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable.

Alternatively, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; 1988; zur Muhlen et al., 1998; Zambaux et al. 1998; Pinto-Alphandry et al., 1995 and U.S. Pat. No. 5,145,684, specifically incorporated herein by reference in its entirety).

### Vaccines

In certain preferred embodiments of the present invention, vaccines are provided. The vaccines will generally comprise one or more pharmaceutical compositions, such as those discussed above, in combination with an immunostimulant. An immunostimulant may be any substance that enhances or potentiates an immune response (including antibody and/or cell-mediated) to an exogenous antigen. Examples of immunostimulants include adjuvants, biodegradable microspheres (e.g., polylactic galactide) and liposomes (into which the compound is incorporated; see, e.g., Fullerton, U.S. Pat. No. 4,235,877). Vaccine preparation is generally described in, for example, Powell & Newman, eds., Vaccine Design (the subunit and adjuvant approach) (1995). Pharmaceutical compositions and vaccines within the scope of the present invention may also contain other compounds, which may be biologically active or inactive. For example, one or more immunogenic portions of other tumor antigens may be present, either incorporated into a fusion polypeptide or as a separate compound, within the composition or vaccine.

Illustrative vaccines may contain DNA encoding two or more of the polypeptides as described above, such that the polypeptides are generated in situ. As noted above, the DNA may be present within any of a variety of delivery systems known to those of ordinary skill in the art, including nucleic acid expression systems, bacteria and viral expression systems. Numerous gene delivery techniques are well known in the art, such as those described by Rolland, Crit. Rev. Therap. Drug Carrier Systems 15:143-198 (1998), and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321 (1989); Flexner et al., Ann. N.Y. Acad. Sci. 569:86-103 (1989); Flexner et al., Vaccine 8:17-21 (1990); U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627 (1988); Rosenfeld et al., Science 252:431-434 (1991); Kolls et al., Proc. Natl. Acad. Sci. USA 91:215-219 (1994); Kass-Eisler et al., Proc. Natl. Acad. Sci. USA 90:11498-11502 (1993); Guzman et al., Circulation 88:2838-2848 (1993); and Guzman et al., Cir. Res. 73:1202-1207 (1993). Techniques for incorporating DNA into such expression systems are well known to those of ordinary skill in the art. The DNA may also be “naked,” as described, for example, in Ulmer et al., Science 259:1745-1749 (1993) and reviewed by Cohen, Science 259:1691-1692 (1993). The uptake of naked DNA may be increased by coating the DNA onto biodegradable beads, which are efficiently transported into the cells. It will be apparent that a vaccine may comprise both a polynucleotide and a polypeptide component. Such vaccines may provide for an enhanced immune response.

It will be apparent that a vaccine may contain pharmaceutically acceptable salts of the polynucleotides and polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

While any suitable carrier known to those of ordinary skill in the art may be employed in the vaccine compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252. One may also employ a carrier comprising the particulate-protein complexes described in U.S. Pat. No. 5,928,647, which are capable of inducing a class I-restricted cytotoxic T lymphocyte responses in a host.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate. Compounds may also be encapsulated within liposomes using well known technology.

Any of a variety of immunostimulants may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium species or Mycobacterium derived proteins. For example, delipidated, deglycolipidated M. vaccae (“pVac”) can be used. In another embodiment, BCG is used as an adjuvant. In addition, the vaccine can be administered to a subject previously exposed to BCG. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); CWS, TDM, Leif, aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

Within the vaccines provided herein, the adjuvant composition may be designed to induce an immune response predominantly of the Th1 type. High levels of Th1-type cytokines (e.g., IFN-γ, TNFα, IL-2 and IL-12) tend to favor the induction of cell-mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within one embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann & Coffman, Ann. Rev. Immunol. 7:145-173 (1989).

Suitable adjuvants for use in eliciting a predominantly Th1-type response include, for example, a combination of monophosphoryl lipid A, for example 3-de-O-acylated monophosphoryl lipid A (3D-MPL), together with an aluminum salt. MPL adjuvants are available from Corixa Corporation (Seattle, Wash.; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352 (1996). Another suitable adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other suitable formulations include more than one saponin in the adjuvant combinations of the present invention, for example combinations of at least two of the following group comprising QS21, QS7, Quil A, β-escin, or digitonin.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as CarbopoIR to increase viscosity, or may be formulated in a dry powder form with a powder excipient such as lactose.

In one embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol containing liposomes, as described in WO 96/33739. Other suitable formulations comprise an oil-in-water emulsion and tocopherol. Another suitable adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 as disclosed in WO 00/09159. Preferably the formulation additionally comprises an oil in water emulsion and tocopherol.

Other suitable adjuvants include Montanide ISA 720 (Seppic, France), SAF (Chiron, Calif., United States), ISCOMS (CSL), MF-59 (Chiron), the SBAS series of adjuvants (SmithKline Beecham, Rixensart, Belgium), Detox (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other suitable adjuvants include adjuvant molecules of the general formula (I):

HO(CH₂CH₂O)ₙ-A-R

wherein, n is 1-50, A is a bond or —C(O)—, R is C₁₋₅₀ alkyl or Phenyl C₁₋₅₀ alkyl.

A further adjuvant of interest is shiga toxin b chain, used for example as described in WO2005/112991.

One embodiment of the present invention consists of a vaccine formulation comprising a polyoxyethylene ether of general formula (I), wherein n is between 1 and 50, preferably 4-24, most preferably 9; the R component is C150, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

Any vaccine provided herein may be prepared using well known methods that result in a combination of antigen, immune response enhancer and a suitable carrier or excipient. The compositions described herein may be administered as part of a sustained release formulation (i.e., a formulation such as a capsule, sponge or gel (composed of polysaccharides, for example) that effects a slow release of compound following administration). Such formulations may generally be prepared using well known technology (see, e.g., Coombes et al., Vaccine 14:1429-1438 (1996)) and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), polyacrylate, latex, starch, cellulose, dextran and the like. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see, e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

Any of a variety of delivery vehicles may be employed within pharmaceutical compositions and vaccines to facilitate production of an antigen-specific immune response that targets tumor cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain embodiments of the present invention use dendritic cells or progenitors thereof as antigen-presenting cells. Dendritic cells are highly potent APCs (Banchereau & Steinman, Nature 392:245-251 (1998)) and have been shown to be effective as a physiological adjuvant for eliciting prophylactic or therapeutic antitumor immunity (see Timmerman & Levy, Ann. Rev. Med. 50:507-529 (1999)). In general, dendritic cells may be identified based on their typical shape (stellate in situ, with marked cytoplasmic processes (dendrites) visible in vitro), their ability to take up, process and present antigens with high efficiency and their ability to activate naïve T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600 (1998)).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allow a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide encoding a protein (or portion or other variant thereof) such that the polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and Cell Biology 75:456-460 (1997). Antigen loading of dendritic cells may be achieved by incubating dendritic cells or progenitor cells with the polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

### Diagnostic Kits

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding a protein in a biological sample. Such kits generally comprise at least one oligonucleotide probe or primer, as described above, that hybridizes to a polynucleotide encoding a protein. Such an oligonucleotide may be used, for example, within a PCR or hybridization assay. Additional components that may be present within such kits include a second oligonucleotide and/or a diagnostic reagent or container to facilitate the detection of a polynucleotide encoding a protein of the invention.

Other diagnostics kits include those designed for the detection of cell mediated responses (which may, for example, be of use in the diagnostic methods of the present invention). Such kits will typically comprise:
(i) apparatus for obtaining an appropriate cell sample from a subject;
(ii) means for stimulating said cell sample with a combination of Chlamydia antigens according to the present invention (or immunogenic fragments thereof, or DNA encoding such antigens or fragments);
(iii) means for detecting or quantifying the cellular response to stimulation.
   Suitable means for quantifying the cellular response include a B-cell ELISPOT kit or alternatively a T-cell ELISPOT kit, which are known to those skilled in the art.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following examples are provided by way of illustration only and not by way of limitation. Those of skill in the art will readily recognize a variety of noncritical parameters that could be changed or modified to yield essentially similar results.

### Example 1

### Expression and Purification of Chlamydia trachomatis Recombinant Proteins

Several Chlamydia trachomatis genes were cloned into plasmid incorporating a 6× histidine tag at the N-terminal to allow for expression and purification of recombinant protein.

Two full-length recombinant proteins, Ct-622 and Ct-875, were expressed in E. coli. Both of these genes were identified using CtL2 and CtE expression screening and the serovar E homologues were expressed. The primers used to amplify these genes were based on serovar L2/E sequences. The genes were amplified using serovar E genomic DNA as the template. Once amplified, the fragments were cloned in pET-17b with a N-terminal 6×-His Tag. After transforming the recombinant plasmid in XL-1 blue cells, the DNA was prepared and the clones fully sequenced. The DNA was then transformed into the expression host BL21-pLysS (Novagen) for production of the recombinant proteins. The proteins were induced with IPTG and purified on Ni-NTA agarose using standard methods. The DNA sequences for CTE622 and CTE875 are disclosed in SEQ ID NO: 9 and 7 respectively, and their amino acid sequences are disclosed in SEQ ID NO: 10 and 8, respectively.

One full-length recombinant protein, Ct-089, was expressed in E. coli. The gene was identified using CtL2 expression screening but the serovar E homologue was expressed. The primers used to amplify this gene was based on serovar L2 sequence. The gene was amplified using serovar E genomic DNA as the template. Once amplified, the fragment was cloned in pET-17b with a N-terminal 6×-His Tag. After transforming the recombinant plasmid in XL-1 blue cells, the DNA was prepared and the clone fully sequenced. The DNA was then transformed into the expression host BL21-pLysS cells (Novagen) for production of the recombinant proteins. The protein was induced with IPTG and purified on Ni-NTA agarose using standard methods.

One full-length recombinant protein, Ct-460, was expressed in E. coli. The gene was identified using CtL2 and CTE expression screening but the serovar L2 homologue was expressed. The primers used to amplify this gene was based on serovar L2 sequence. The genes were amplified using serovar L2 genomic DNA as the template. Once amplified, the fragment was cloned in pET-17b with a N-terminal 6×-His Tag. After transforming the recombinant plasmid in XL-1 blue cells, the DNA was prepared and the clone fully sequenced. The DNA was then transformed into the expression host BL21-pLysE cells (Novagen) for production of the recombinant proteins. The protein was induced with IPTG and purified on Ni-NTA agarose using standard methods.

One full-length recombinant protein, Ct-858, was expressed in E. coli. The gene was identified using CtL2 and CTE expression screening but the serovar E homologue was expressed. The primers used to amplify this gene was based on serovar L2/E sequence. The genes were amplified using serovar E genomic DNA as the template. Once amplified, the fragment was cloned in pCRX2 with a N-terminal 6×-His Tag. After transforming the recombinant plasmid in XL-1 blue cells, the DNA was prepared and the clone fully sequenced. The DNA was then transformed into the expression host Tuner DE3 cells (Novagen) for production of the recombinant proteins. The protein was induced with IPTG and purified on Ni-NTA agarose using standard methods.

One full-length recombinant protein, Ct-681, was expressed in E. coli. The gene was identified using CtL2 and CTE expression screening but the serovar F homologue was expressed. Clone/pET-15-construct was obtained from GSK (MompF). Once amplified, the fragment was cloned in pET-15b with a N-terminal 6×-His Tag. After transforming the recombinant plasmid in XL-1 blue cells, the DNA was prepared and the clone fully sequenced. The DNA was then transformed into the expression host BL21-pLysS cells for production of the recombinant proteins. The protein was induced with IPTG and purified on Ni-NTA agarose using standard methods.

The passenger domain of two recombinant proteins, Ct-812 and Ct-871, were expressed in E. coli. Ct-812 was identified using CtL2 and CtE expression screening and Ct-871 was identified using CtE expression. For both genes the serovar L2 homologues were expressed. The primers used to amplify these genes were based on serovar L2 sequences. The genes were amplified using serovar L2 genomic DNA as the template. Once amplified, the fragments were cloned in pET-17b with a N-terminal 6×-His Tag. After transforming the recombinant plasmid in XL-1 blue cells, the DNA was prepared and the clones fully sequenced. The DNA was then transformed into the expression host BL21-pLysS cells (Novagen) for production of the recombinant proteins. The proteins were induced with IPTG and purified on Ni-NTA agarose using standard methods.

### Example 2

### Formulation of Five Different Combinations of Chlamydia trachomatis Antigens with Adjuvant

The antigen combinations in the table below were prepared as follows. 5 μg of each antigen was combined in 50 μl of PBS and then mixed with 50 □l AS01B adjuvant which comprises 3D-MPL and QS21 formulated with cholesterol containing liposomes, to a total volume per dose of 100 μl.

After mixing with the antigen the final composition of the adjuvant is:

| | | |
|---|---|---|
| 3D-MPL | 100 | ug/ml |
| QS21 | 100 | ug/ml |
| DOPC | 2 | mg/ml |
| Cholesterol | 0.5 | mg/ml |

| | Swib | Momp | | | | | PmpGpd | PmpDpd |
|---|---|---|---|---|---|---|---|---|
| COMBO | CT460 | CT681 | Ct-858 | Ct-875 | Ct-622 | Ct-089 | CT871 | CT812 |
| 1 | X | X | X | | | X | | X |
| 1′ | X | | X | | | X | | X |
| 2 | X | X | X | | X | X | | X |
| 3 | | X | X | | X | X | X | X |
| 4 | | | X | X | X | X | | |
| 5 | | | X | X | | X | | |
| 5′ | | | X | X | | X | | X |
| 6 | | X | X | | | X | X | X |

### Example 3

### Testing of Combinations of Chlamydia trachomatis Antigens in a Mouse Model—Immunization Against Chlamydia Genital Tract Infection

This example demonstrates that vaccination with Chlamydia antigen combinations as described in Example 2 can significantly protect against Chlamydia infection in mice.

A murine model of genital tract infection with human serovar K strain of Chlamydia trachomatis (Ct) was developed that closely resembles the pathology of infection in humans. This model was used to evaluate the effectiveness of immunizing mice with a number of combinations of Ct-specific antigens from different serovars. Specifically, Balb/c mice and C57Bl/6 mice were vaccinated with formulations of adjuvant combinations as described in Example 2. This model was also attempted with a third mouse strain, DBA, but this model did not allow protection against Ct challenge to be demonstrated either in the positive control (UV irradiated chlamydial elementary bodies (UVEB) formulated in AS01B) or in mice vaccinated with the antigen combinations.

Two injections, separated by a three week time interval, were administered to the mice at the base of the tail. Four weeks following the final vaccination, the animals were treated with 1.25 mg of progesterone prior to being intra-vaginally infected with 5×10⁵ Inclusion Forming Units (IFU) of purified Chlamydia trachomatis, serovar K. Mice were immunized with 10 μg UVEB formulated in AS01B as a positive control and the adjuvant AS01B alone as a negative control. Seven days post-infection, the lower genital tracts were swabbed to determine bacterial shedding values by determining IFU using McCoy cells. In some experiments mice were sacrificed at day 10 post-infection and bacterial load in the upper genital tract was determined by homogenizing the UGT and determining IFU using McCoy cells.

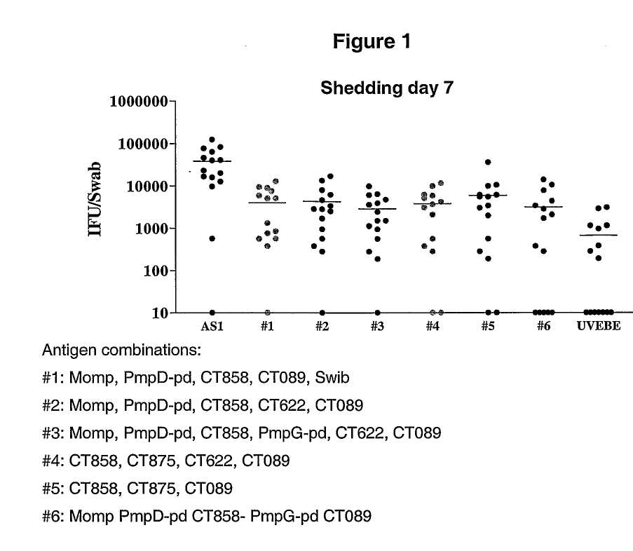

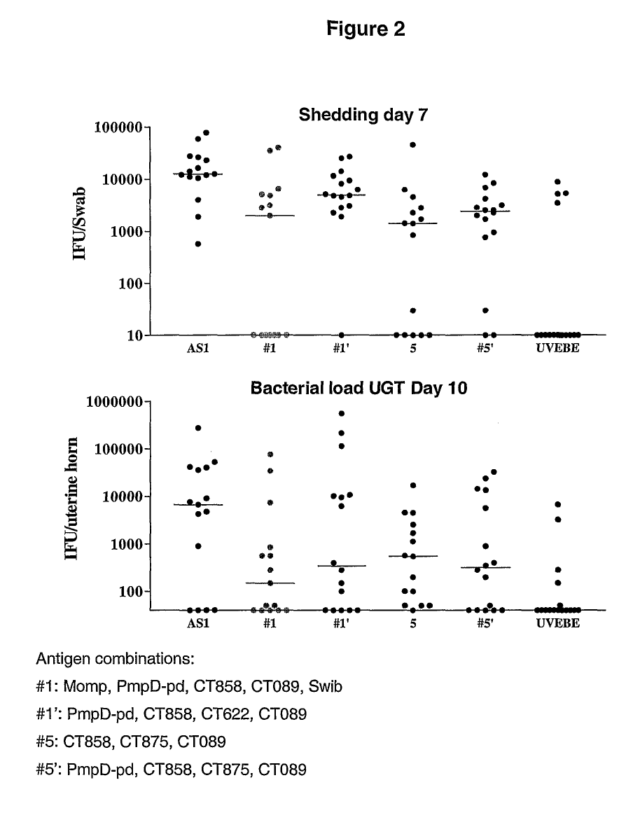

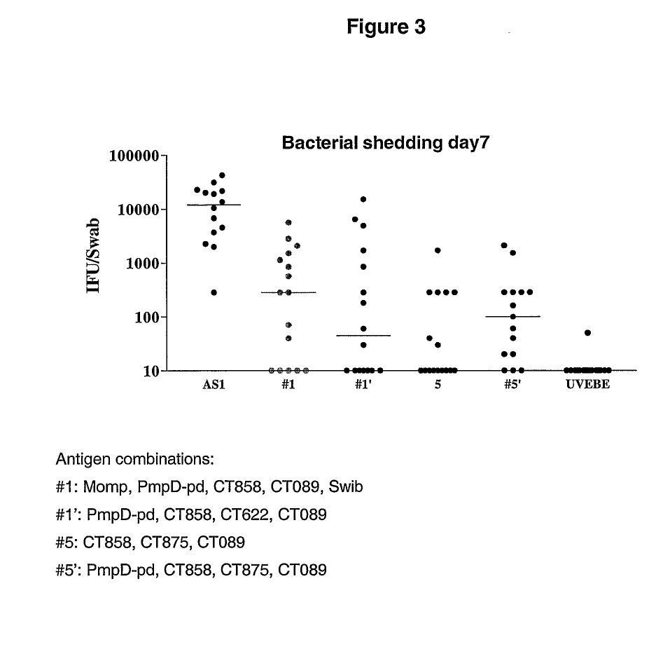

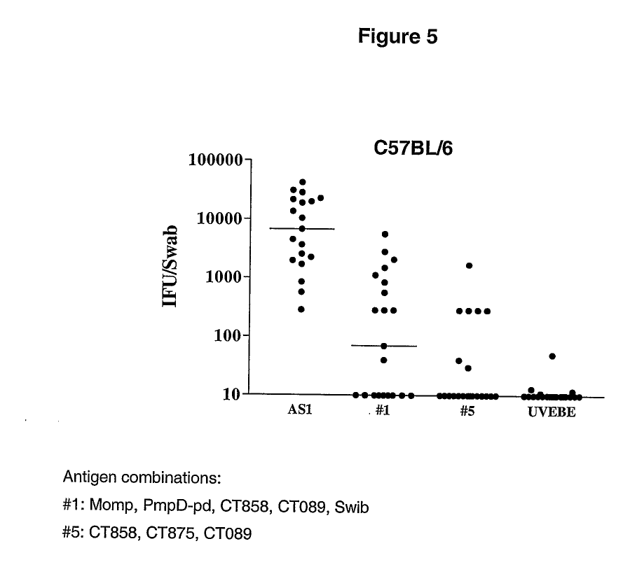

As shown in FIGS. 1 and 2, vaccination of mice with combinations 1, 1′, 2, 3, 4, 5, 5′ or 6 shows the surprising result of offering significant protection (p<0.01-p<0.001) against Chlamydia infection in a Balb/c mouse model. Furthermore, as shown in FIGS. 3 and 5 the protection results are confirmed in a second mouse protection model, C57Bl/6, vaccinated with combinations 1, 1′, 5 and 5′ and challenged with serovar K. (p<0.001 Dunnet's multiple comparison test).

For better statistical analysis the day 7 shedding data from three experiments in Balb/c mice were pooled (FIG. 1). The mean bacterial shedding in the UVEB-immunized groups was reduced by 1.8 log compared to the mean of the AS01B control group. The mean bacterial shedding in the UVEB-immunized groups was significantly lower when compared to all other tested groups (two-tailed t-test, p<0.05). All antigen combinations significantly reduced the mean of bacterial shedding by approximately one log (0.8-1.1) when compared to the AS01B control group (p<0.01; Dunnett's multiple comparison test). The statistical analysis did not detect any difference in the protection induced by the 6 antigen combinations (Tukey and t-test). Thus, immunizations of Balb/c mice with antigen combinations tested induced significant protection against bacterial shedding in the vaginal challenge model with serovar K.

Next, we initiated a set of back to back confirmation experiments in Balb/c and C57Bl/6 mice comparing the combinations 1 and 5 to the two modified versions of combination 1 and 5, combination 5′(adding PmpD-pd to combination 5) and combination 1′(taking MompF out of combination 1). Groups of 8 progesterone-treated Balb/c or C57Bl/6 mice were challenged with an intra-vaginal dose of 5×10⁵ IFU of serovar K four weeks after the second immunization. The data for experiments in Balb/c mice are shown in FIG. 2. Bacterial shedding was determined from swabs taken on day 7 post chlamydial challenge. Mice were sacrificed on day 10 to determine the chlamydial load in the UGT. The data of these experiments have been pooled together for statistical analysis (FIG. 2). UVEB immunization protected 12 out of 16 mice completely against bacterial shedding in the lower genital tract (day 7 post challenge) and there were no Chlamydia detected in the UGT of 11 out of the 16 mice on day 10 post challenge. The medians of both, the bacterial shedding in the LGT and the bacterial load in the UGT, were reduced by at least 1 log in mice immunized with combination 1 or combination 5 when compared to the AS01B-only control. The modification (increase or reduction in number of antigens) of the composition of combination 1 and combination 5 (combination 1′ and combination 5′) did not improve protection. There were statistically significant differences between the means of bacterial shedding of all the groups when compared to the mean of the AS01B control group using the Dunnet's multiple comparison test with the following p values:
AS01B vs. UVEB p<0.001
AS01B vs. combination 5 p<0.001
AS01B vs. combination 5′ p<0.001
AS01B vs. combination 1 p<0.001
AS01B vs. combination 1′ p<0.05

Like in the previous confirmation experiments in Balb/c mice, the statistical analysis has not allowed to distinguish between the antigen combinations. Statistical analysis of the bacterial load in the UGT determined that only the median of the UVEB immunized group was significant lower than the median of the AS01B control group.

The bacterial shedding for the back to back experiments with combinations 1 and 5 in C57Bl/6 mice are shown in FIG. 3. The data from the back to back experiments were pooled for statistical analysis. The experiments followed the Balb/c protocol. Bacterial shedding was determined from swabs taken on day 7 post chlamydial challenge. Immunization with the antigen combination 1, 1′, 5 and 5′ induced significant protection against shedding by 1 to 2 logs, respectively (p<0.001; Dunnet's multiple comparison test). The statistical analysis did not determine any statistical significance between the bacterial shedding means of mice immunized with the combos.

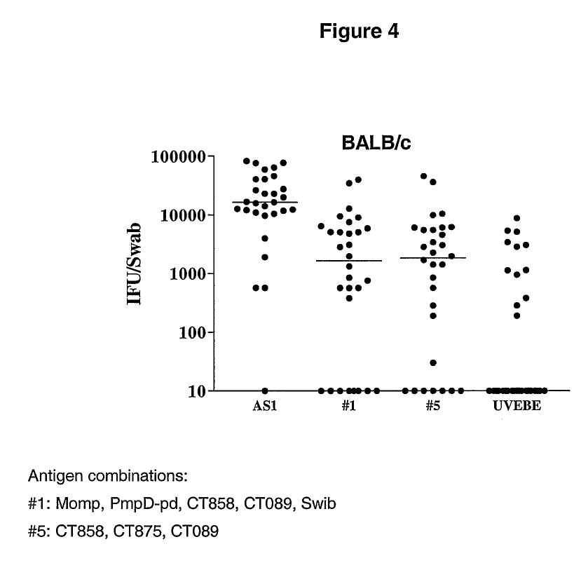

For a final statistical analysis the data from the five confirmation experiments in Balb/c (31 mice per group) and three confirmation experiments in C57Bl/6 mice (21 mice per group) were pooled and are shown in FIGS. 4 and 5. The confirmation experiments demonstrated that the selected combinations 1 and 5 induce significant protection against bacterial shedding in both Balb/c (p<0.001) and C57Bl/6 mice (p<0.001). The data confirm that antigen combinations identified in the experimental design experiment in Balb/c mice also induce protection in C57Bl/6 mice. The data also show that immunizations with these combinations as well as immunizations with UVEB induce higher protection levels in C57Bl/6 mice than in Balb/c mice.

### Example 4

### Ct-089, Ct-858 and Ct-875 Sequence Comparisons

### Method

Chlamydia trachomatis serovar E is a common oculogenital serovar and was chosen as a basis to which the other sequences would be compared.

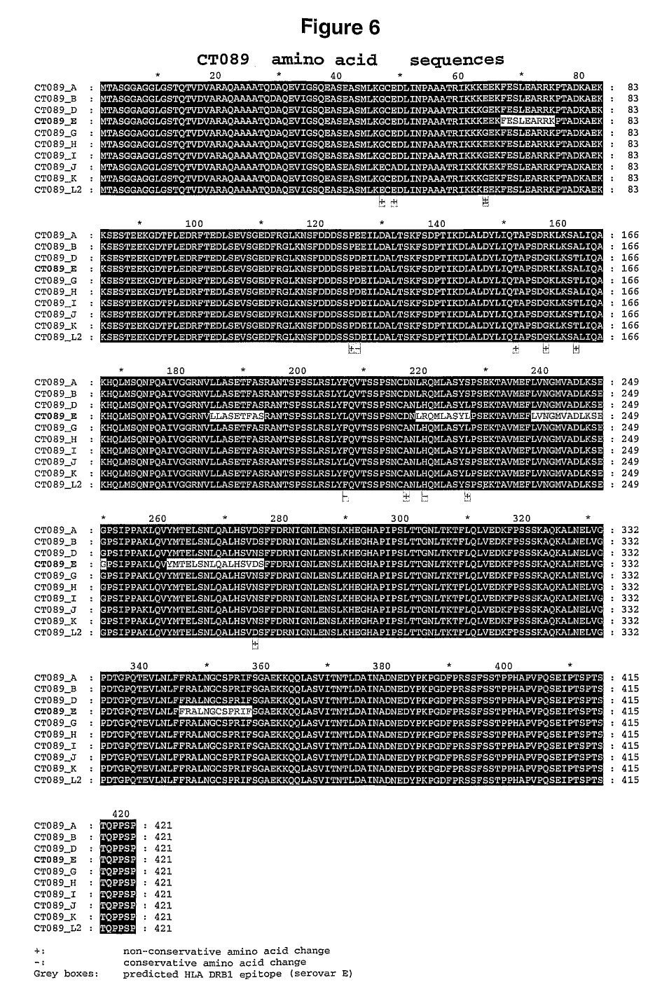

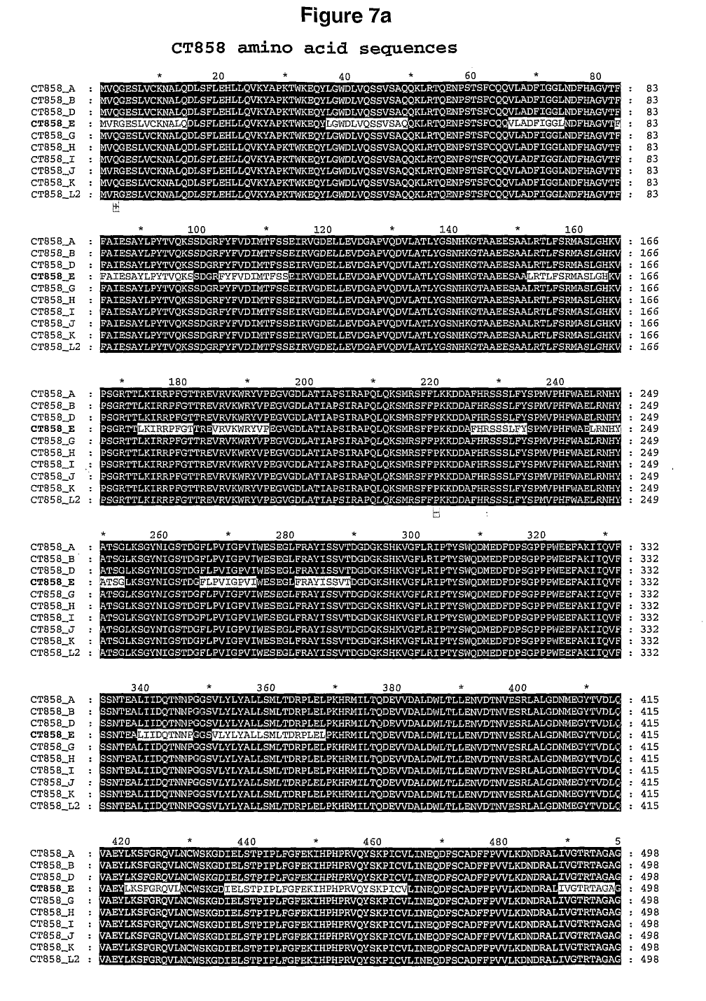

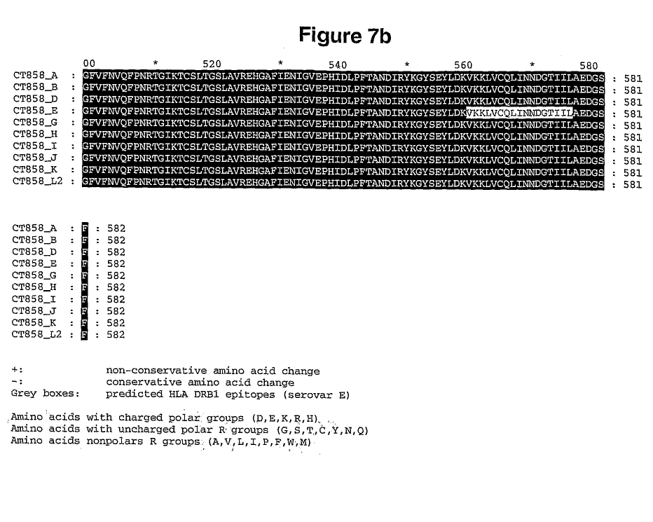

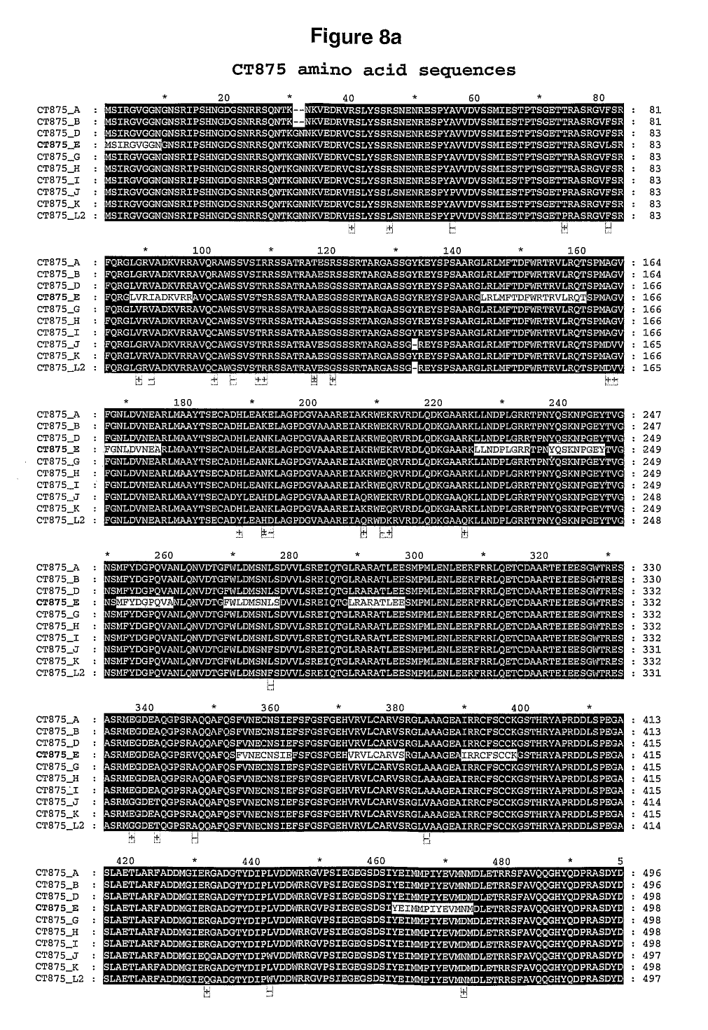

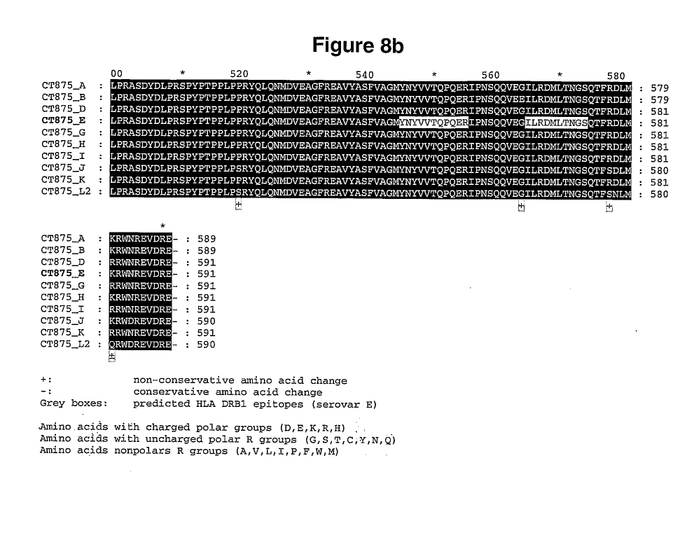

A multiple alignment of amino-acid sequences for comparison has been conducted using the CLUSTAL W program, available in the Lasergene software package, version 5.0 (sold by DNASTAR, Inc., Madison, Wis.)). The basic multiple alignment algorithm involves a three-step procedure: all pairs of sequences are aligned separately in order to calculate a distance matrix giving the divergence of each pair of sequences, then a guide tree is calculated from the distance matrix and finally the sequences are progressively aligned according to the guide tree. CLUSTAL W algorithm is described in Thompson et al., Nuc. Acids Res. 22: 4673-4680 (1994). The alignments are shown in FIGS. 6, 7a/7b and 8a/8b.

The T-helper cell epitopes are peptides bound to HLA class II molecules and recognized by T-helper cells. The prediction of putative T-helper cell epitopes, present on CT089, CT858 and CT875 Chlamydia trachomatis polypeptides from serovar E, was based on the TEPITOPE method described by Sturniolo et al., Nature Biotech. 17:555-561 (1999). The peptides comprising good, potential T-cell epitopes are highlighted (grey boxes) in FIGS. 6, 7a/7b and 8a/8b.

### Results

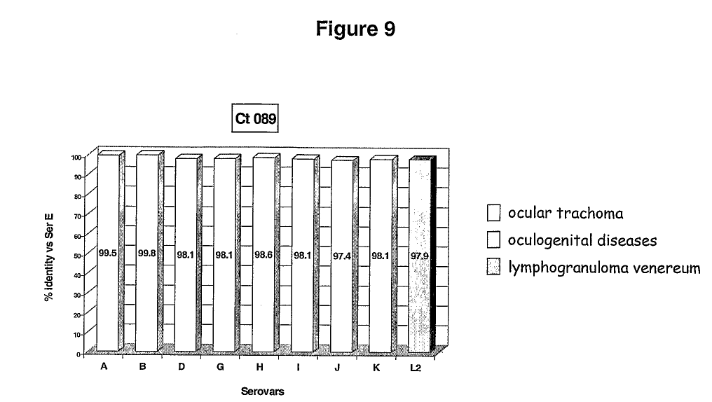

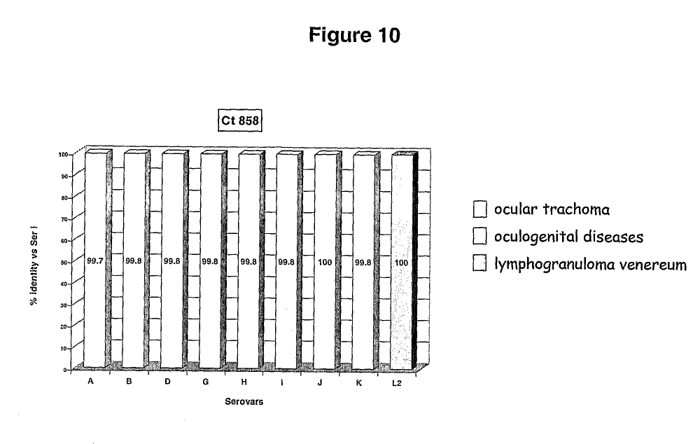

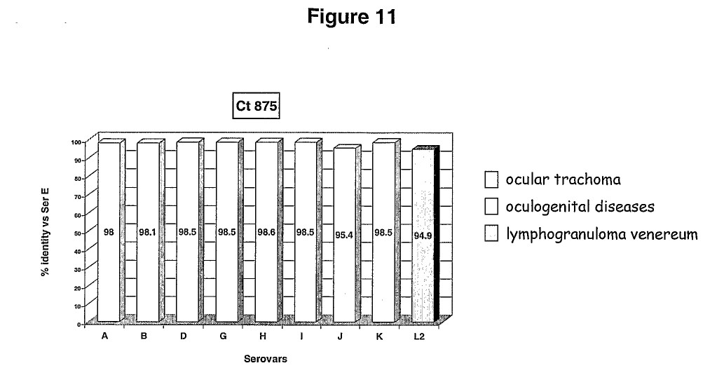

FIG. 9 shows the results of comparison of Ct-089 sequences. FIG. 10 shows the results of comparison of Ct-858 sequences. FIG. 11 shows the results of comparison of Ct-875 sequences.

Ct-089 from Chlamydia trachomatis serovar E shows a high level of sequence identity to Ct-089 from Chlamydia trachomatis serovars A, B, D, G, H, I, J, K and L2. The minimum level of identity was 97.4%, with eight of the ten serovars having at least 98% identity. The ocular serovars A and B show particularly high identity to serovar E, with values of 99.5% and 99.8% respectively.

Ct-858 from Chlamydia trachomatis serovar E shows a very high level of sequence identity to Ct-858 from Chlamydia trachomatis serovars A, B, D, G, H, I, J, K and L2. The minimum level of identity was 99.7%, with ocular serovar J and LGV serovar L2 showing complete identity.

Ct-875 from Chlamydia trachomatis serovar E shows a high level of sequence identity to Ct-089 from Chlamydia trachomatis serovars A, B, D, G, H, I, J, K and L2. The minimum level of identity was 94.9%, with eight of the ten serovars having at least 98% identity.

For each of the three proteins Ct-089, Ct-858 and Ct-875, the percentage of HLA DRB1 predicted epitopes (for serovar E) which are fully conserved across all of the serovars tested is very high and estimated at 77%, 95% and 80%, respectively.

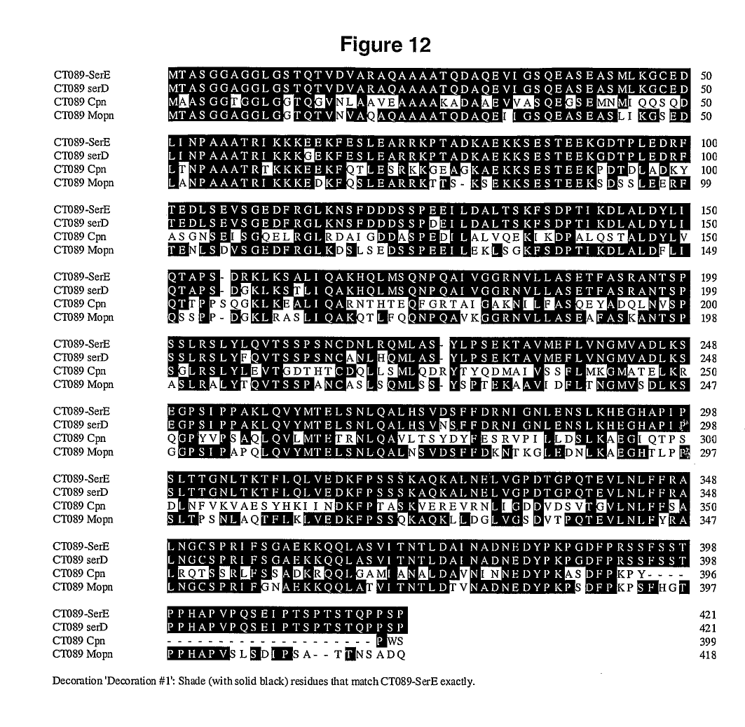

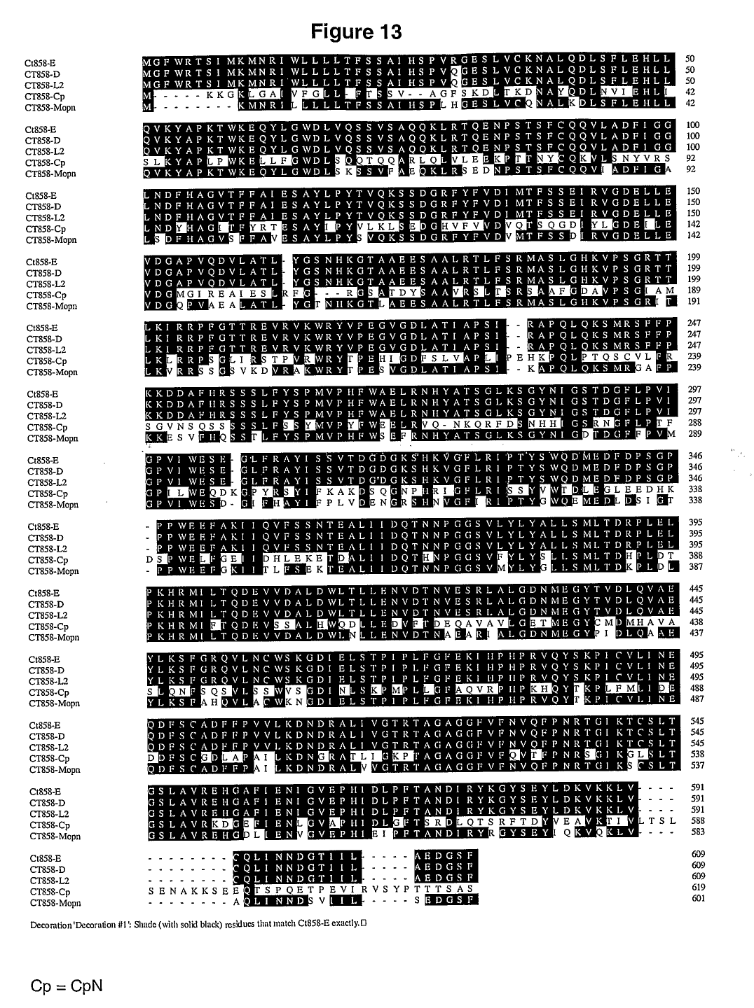

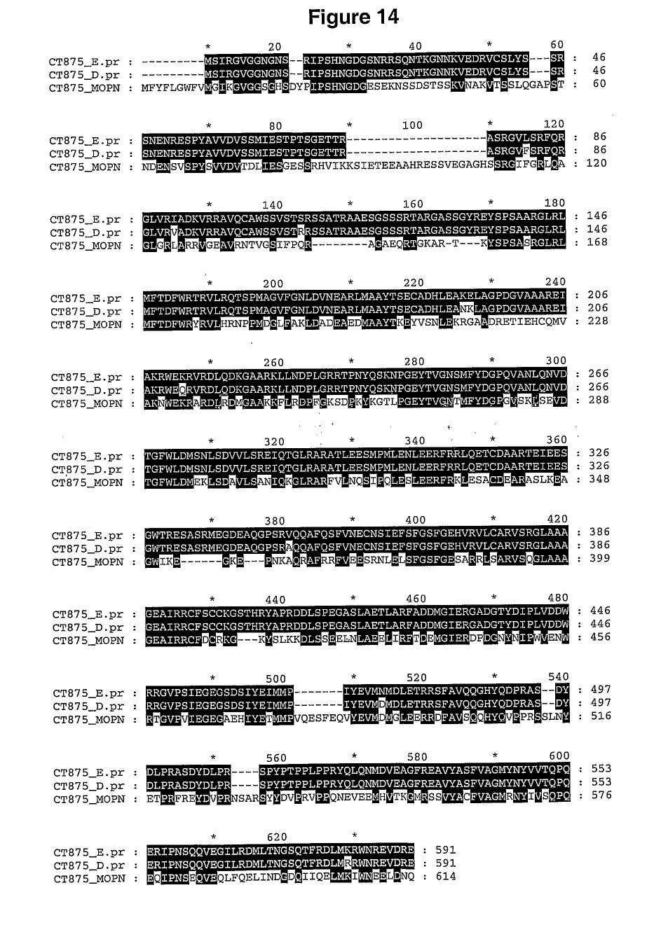

For comparative purposes, FIGS. 12, 13 and 14 show the sequence alignment for Ct-089, Ct-858 and Ct-875 from serovar E respectively with their equivalents from other Chlamydia trachomatis serovars and other Chlamydia species. Cpn indicates the corresponding sequence from Chlamydia pneumoniae, MoPn indicates the corresponding sequence from Chlamydia muridarum.

Ct-089 (Serovar E) amino acid identity

| | |
|---|---|
| Ct-089 (Serovar D) | 99.8% |
| C. pneumoniae - CpN | 47.4% |
| (SEQ ID No 78) | |
| C. muridarum - MoPn | 73.7% |
| (SEQ ID No 74) | |

Ct-858 (Serovar E) amino acid identity

| | |
|---|---|
| Ct-858 (Serovar D) | 99.8% |
| Ct-858 (Serovar L2) | 99.8% |
| C. pneumoniae - CpN | 44.2% |
| (SEQ ID No 40) | |
| C. muridarum - MoPn | 82.2% |
| (SEQ ID No 36) | |

Ct-875 (Serovar E) amino acid identity

| | |
|---|---|
| Ct-875 (Serovar D) | 98.5% |
| C. muridarum - MoPn | 52.3% |
| (SEQ ID No 24) | |

### Conclusion

In summary, each of the three proteins Ct-089, Ct-858 and Ct-875 have highly conserved sequences across all of the Chlamydia trachomatis serovars tested.

Furthermore, the data indicates that there is no link between the degree of sequence variation and disease state associated with a particular serovar. For example, in the case of Ct-089, the oculogenital serovar E shows the highest homology to the ocular serovars A and B, while in the case of Ct-858, serovar E shows the highest homology to the oculogenital serovar J and LGV serovar L2.

Sequence homology of Ct-089, Ct-858 and Ct-875 with the equivalent proteins in other Chlamydia species is relatively low.

The antigenic properties of Ct-089, Ct-858 and Ct-875 have already been described in the prior art. However, contrary to the expectation of one skilled in the art, as a result of the low sequence variation, vaccines containing Ct-089, Ct-858 and Ct-875, immunogenic fragments thereof or polynucleotides encoding them, and which are derived from a first Chlamydia trachomatis serovar may be expected to be of use in the treatment or prevention of Chlamydial infection by a second Chlamydia trachomatis serovar.

### Example 5

### Purification of Chlamydia trachomatis Elementary Bodies from Servars D, E, J and K

Purified elementary bodies were required for challenge of vaccine test subjects and for the preparation of UV irradiated elementary bodies (UVEB) which are used as a positive control vaccine in later examples.

### Method

EB from each of the Chlamydia trachomatis serovars were prepared. Briefly, all serovars were grown separately in confluent McCoy cell monolayers and cultured in RPMI medium (75 cm² culture flasks) that was supplemented with 1 μg/ml of cycloheximide immediately before inoculation. Flasks were inoculated with non-purified lysates from infected cells containing 106 to 10⁷ Infectious Forming Units (IFU) in Sucrose Phosphate Glutamic Acid (SPG). Flasks were spun at 2000 rpm for 1 hour in a table-top cell culture centrifuge and then incubated for 48 or 72 hours at 37° C. in a CO₂ atmosphere. This process was repeated until there were at least 20 flasks of highly infected cell populations (>80% of cells were infected) ready for purification. Chlamydia elementary bodies were purified by ultracentrifugation over a series of Hypaque gradients (30%, 52%, 44% and 40%) with intervening washes in SPG.

### Results

The titer of the purified EB for each Chlamydia trachomatis serovar was assessed using the Chlamydia titration infectivity assay and immunofluorescence microscopy (using FITC-conjugated anti-C. trachomatis antibody and Evan's Blue in PBS) to calculate the number of IFU per ml. Titers for the resulting purified EB were found to range from 1.2×10⁶ to 2.6×10⁹ IFU/ml

### Example 6

### Expression and Purification of Ct-089, Ct-858 and Ct-875 Proteins

To prepare the test vaccines, stocks of purified Chlamydia trachomatis serovar E for use in later examples Ct-089, Ct-858 and Ct-875 proteins were prepared by expressing their genes in E. coli.

### Method

Competent E. coli strains BL21 plys E, Tuner (DE3) and BL21 plys S were transformed with Ct-089, Ct-858 and Ct-875 expression plasmids respectively and grown on the appropriate antibiotic selection medium. The resulting expression clones were used in a mini-induction protocol, and protein yields analyzed by SDS-PAGE. If cells grew well during this process and proteins were induced by isopropyl-beta-D-thiogalactopyranoside (IPTG) in sufficient quantities to be detected on Coomassie blue-stained SDS gels, the clones were used in a large-scale induction experiment (IPTG, 1 mM).

Following lysis of cells in a CHAPS solution and centrifugation, aliquots of the soluble and pellet fractions were analyzed by SDS-PAGE to determine whether the majority of the protein of interest was in the pellet or soluble fraction. The fraction containing the majority of each antigen was subjected to Ni-NTA column purification (after appropriate solubilisation of proteins). Aliquots of the preparations, including material from before Ni-NTA binding, column flow-through, column washes, and column elution fractions, were analyzed by SDS-PAGE. Fractions containing the eluted protein were combined, dialyzed against 10 mM Tris pH 8 or pH 10, filtered sterilized, and concentrated. The BCA protein assay was used on the concentrated CT protein fractions, and purity was assessed by SDS-PAGE.

### Example 7

### Evaluation of the Protection Induced by a Vaccine Containing Ct-089, Ct-858 and Ct-875 from Chlamydia trachomatis Serovar E Against Challenge with Chlamydia trachomatis Serovars D, K and J

The protection provided by a vaccine containing Ct-089, Ct-858 and Ct-875 Chlamydia trachomatis serovar E antigens was tested in vaginal challenge experiments with EB from heterologous (i.e. non-serovar E) Chlamydia trachomatis serovars.

### Method

The study was conducted with 63 six-week old female C57Bl/6 mice. These mice were split into three groups of twenty-one mice, each group to be challenged by a different serovar (Chlamydia trachomatis serovar D, K, or J). The groups were then further separated into sub-groups of seven mice each. These three sub-groups were immunised intramuscularly with 50 ul of different vaccine preparations injected into each anterior tibialis (100 ul total), and repeated three weeks later. Mice were further treated with progesterone, 1.25 mg given in a volume of 100 ul by subcutaneous injection ten and three days before challenge to synchronise their cycles. The three test preparations were:
(i) Adjuvant control (AS01B)
The adjuvant utilised was based upon a liposomal formulation containing 3D-MPL, QS21 and cholesterol. The final composition of the adjuvant solution being:

| | | |
|---|---|---|
| 3D-MPL | 100 | ug/ml |
| QS21 | 100 | ug/ml |
| DOPC | 2 | mg/ml |
| Cholesterol | 0.5 | mg/ml |

Phosphate buffered saline was prepared from 9 mM Na₂HPO₄, 48 mM KH₂PO₄ and 100 mM NaCl at pH 6.1.
A mixture of lipid, cholesterol and 3D-MPL was prepared in organic solvent, this was then dried under vacuum. PBS was then added and the vessel agitated until a suspension formed. This suspension was then microfluidised until a liposome size of around 100 nm was obtained (referred to as small unilamellar vesicles or SUV). Subsequently, the SUV were sterilized by passage through a 0.2 um filter.
Sterile SUV were mixed with the appropriate quantity of aqueous QS21 (at a concentration of 2 mg./ml) with the addition of phosphate buffered saline to obtain the final desired concentrations. The pH was then adjusted to 6.1 (+/−0.1) as necessary using sodium hydroxide or hydrochloric acid.
(ii) UV attenuated Chlamydia trachomatis elementary bodies with AS01B adjuvant
A preparation containing 10 ug of UV treated Chlamydia trachomatis elementary bodies (UVEB) from serovar E with adjuvant (as described above).
(iii) Ct-089, Ct-858 and Ct-875 with AS01B adjuvant
A preparation containing Ct-089 (5 ug), Ct-858 (5 ug) and Ct-875 (5 ug) from Chlamydia trachomatis serovar E with adjuvant (as described above).

Mice were challenged, under anaesthetic (1:1 Ketaject and Xylaject), four weeks after final boost with 1×10⁶ IFU of serovar D, K or J suspended in 20 ul of sucrose phosphate glutamic acid (SPG).

The infection was allowed to proceed for 10 days, with genital swabs were taken under anaesthetic on Day 4 and Day 7. Mice were euthanized on Day 10 and the uterine horns harvested for histopathology and titration. For titration, one-half of the UGT was homogenized, and IFU was determined using McCoy cells.

Samples (vaginal swabs) collected from days 4, 7, and 10 post-challenge were thawed at 37° C. A small amount of glass beads (Sigma) was added to each sample and vortexed for five minutes in 1 ml of SPG. 100 μl of each sample was inoculated onto a monolayer of McCoy cells in medium containing 1 μg/ml cyclohexamide in a 24-well plate. Plates were spun at 2000 rpm for one hour before being transferred to a 37° C. incubator. Time of incubation is 48-72 hours before fixation.

After incubation, methanol that had been pre-chilled at −20° C. was used to fix the cells. Each well was filled with methanol and left at −20° C. for at least 10 minutes. Plates were then washed with PBS three times before staining with Goat anti-chlamydia trachomatis FITC conjugated polyclonal antibody (Chemicon). The stain solution consisted of Evan's Blue Stain (Sigma), FITC-conjugated anti-C. trachomatis antibody, and PBS. Evan's Blue stain was diluted 1:200 in PBS, and FITC-conjugated anti-C. trachomatis antibody was diluted at 1:100. 500 μl of the stain solution was added to each well. Plates were then incubated at 37° C. for 1.5-2 hours.

After the incubation period, the stain was aspirated and the plates were washed with PBS five times on a rocking platform, each time for at least 5 minutes. After the final wash, 1 ml of PBS was added to each well, and the plates were ready to be titered.

There were three methods used for calculating the number of IFU per swab. The primary way consisted of counting 10 random fields under a fluorescence microscope and then using the following formula (s):
n×10×190 (using objective lens 10×)
n×10×283 (using objective lens 20×)
n×10×1180 (using objective lens 40×)
   where n=mean of inclusion bodies counted in 10 random fields, 10 is the dilution factor, and 190, 283 and 1180 are the respective focal conversion factors.

The following method was used when low numbers of inclusion bodies were seen in an entire well:

s×10

where s=number of inclusion bodies counted in a well and 10 is the dilution factor.

Finally, when no inclusion bodies were seen, an arbitrary value of 7 was chosen to represent IFU/swab. This was based on the assumption that although no inclusion bodies were detected in a tenth of the swab, that did not necessarily mean that there were no inclusion bodies in the entire swab.

### Results

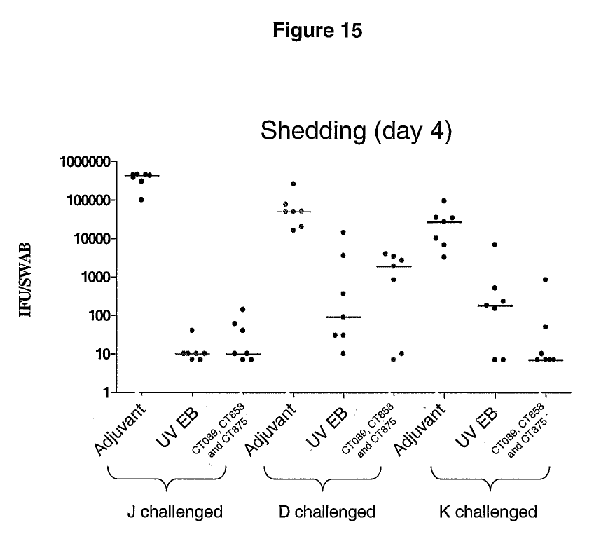

FIGS. 15 to 17 illustrate the results of Example 7.

FIG. 15 indicates that four days after challenge, mice immunised with Ct-089, Ct-858 and Ct-875 according to the present invention show lower levels of shedding compared to the adjuvant control. Furthermore, levels of shedding are generally comparable to those achieved with immunisation by UVEB (lower levels are achieved in the case of serovar K challenge, and higher levels in the case of serovar D challenge).

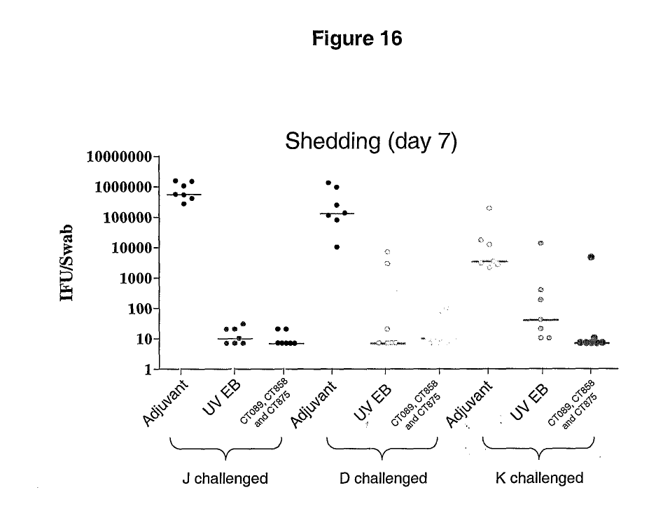

Seven days after challenge mice receiving the adjuvant control show higher levels of shedding than those immunised with UVEB or with Ct-089, Ct-858 and Ct-875 (see FIG. 16). Both UVEB and Ct-089, Ct-858 and Ct-875 immunised mice show very low levels of shedding.

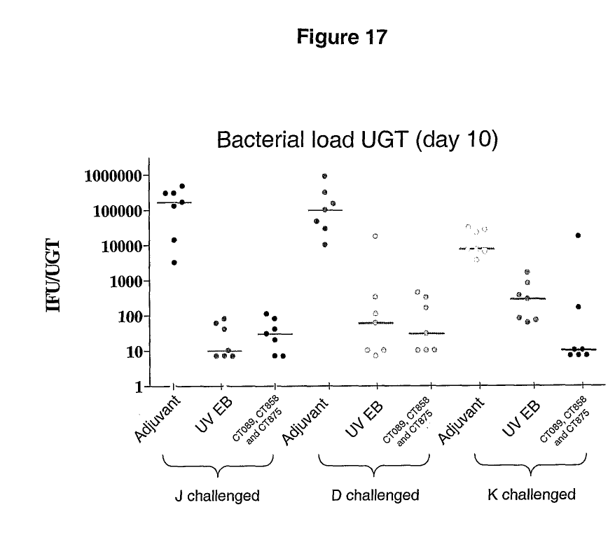

FIG. 17 shows that ten days after challenge the UGT of mice receiving the adjuvant control is highly colonised by bacteria. Both UVEB and Ct-089, Ct-0858 and Ct-875 immunised mice show low levels of UGT colonisation, with treatment according to the invention generally showing a slightly lower level than UVEB treatment.

Statistical analysis indicates that treatment using the Ct-089, Ct-0858 and Ct-875 antigens from Chlamydia trachomatis serovar E results in significant protection in mice challenged with Chlamydia trachomatis serovar J when compared to the negative control (adjuvant only) with p<0.01 by Dunnett's multiple comparison test. No significant difference is seen between antigen treatment and UVEB treatment (p>0.05).

Treatment using the Ct-089, Ct-858 and Ct-875 antigens from Chlamydia trachomatis serovar E results in significant protection in mice challenged with Chlamydia trachomatis serovar D on Day 4 and Day 7 when compared to the negative control (adjuvant only) with p<0.05. No significant difference is seen between antigen treatment and UVEB treatment (p>0.05).

Treatment using the Ct-089, Ct-858 and Ct-875 antigens from Chlamydia trachomatis serovar E results in significant protection in mice challenged with Chlamydia trachomatis serovar K on Day 4 and Day 10 when compared to the negative control (adjuvant only) with p<0.05 and p<0.01 respectively. No significant difference is seen between antigen treatment and UVEB treatment (p>0.05).

### Conclusions

The experiments performed in Example 7 confirm that the combination of the three proteins, Ct-089, Ct-858 and Ct-875 from Chlamydia trachomatis serovar E is capable of eliciting a substantial protective response, which has been shown to be statistically significant. This protective response is one which can provide general protection against challenge by other serovars. In particular, it should be noted that serovars E and K are the most genetically diverse Chlamydia trachomatis serovars and the cross-protection observed between these two Chlamydia trachomatis serovars suggests that a combination of Ct-089, Ct-858 and Ct-875 antigens may be expected to have wide use in the treatment or prevention of Chlamydial infection.

The level of protection afforded by the vaccine formulation containing Ct-089, Ct-858 and Ct-875 was found to be comparable to the use of UVEB, although clearly the use of purified proteins is desirable in light of the risks involved with the use of the whole elementary bodies.

### Example 8

### Evaluation of the Protection Induced by a Vaccine Containing Ct-089, Ct-858 and Ct-875 from Chlamydia trachomatis Serovar E Against Challenge with Serovars K and J, in the Highly Susceptible Mouse Strain C3H/HeN

The protection provided by a vaccine containing Ct-089, Ct-858 and Ct-875 serovar E antigens was tested in vaginal challenge experiments with EB from heterologous (i.e. non-serovar E) Chlamydia trachomatis serovars K and J.

### Method

The study was conducted with 41 fifteen-week old female C3H/HeN mice, a strain known for their susceptibility to chlamydial infection. These mice were split into two groups, one of which was challenged by Chlamydia trachomatis serovar K and the other by serovar J. The groups were then further separated into three sub-groups, these sub-groups were immunised intramuscularly with 50 ul of different vaccine preparations injected into each anterior tibialis (100 ul total), and repeated four weeks later. Each sub-group contained seven mice, save for the group immunised with adjuvant control and challenged with serovar K which contained 6 mice. Mice were further treated with progesterone, 1.25 mg given in a volume of 100 ul by subcutaneous injection ten and three days before challenge to synchronise their cycles. The three test preparations were:
(i) Adjuvant control (AS01B)
The adjuvant utilised was based upon a liposomal formulation containing 3D-MPL, QS21 and cholesterol. The final composition of the adjuvant solution being:

| | | |
|---|---|---|
| 3D-MPL | 100 | ug/ml |
| QS21 | 100 | ug/ml |
| DOPC | 2 | mg/ml |
| Cholesterol | 0.5 | mg/ml |

Adjuvant was prepared as described above in Example 7.
(ii) UV attenuated Chlamydia trachomatis elementary bodies with AS01B adjuvant
A preparation containing 10 ug of UV treated Chlamydia trachomatis elementary bodies (UVEB) from serovar E with adjuvant (as described above).
(iii) Ct-089, Ct-858 and Ct-875 with AS01B adjuvant
A preparation containing Ct-089 (5 ug), Ct-858 (5 ug) and Ct-875 (5 ug) from Chlamydia trachomatis serovar E with adjuvant (as described above).

Mice were challenged, under anaesthetic (1:1 Ketaject and Xylaject, 30 ul per mouse IP, 20 ul into each thigh), four weeks after final boost with 1×10⁶ IFU of serovar K or J suspended in 20 ul of sucrose phosphate glutamic acid (SPG).

The infection was allowed to proceed for 10 days, with genital swabs were taken under anaesthetic on Day 4 and Day 7. Mice were euthanized on Day 10 and the uterine horns harvested for histopathology and titration. For titration, one-half of the UGT was homogenized, and IFU was then determined using McCoy cells as described in Example 7.

The detection limit for titering is 10 IFU, thus one inclusion per well is plotted as 10 IFU. An arbitrary number of 7 IFU was allocated where the number of inclusions observed was less than one.

### Results

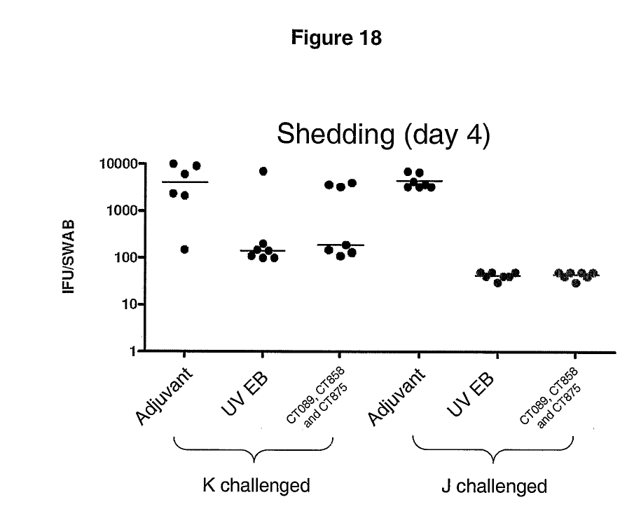

FIGS. 18 to 20 illustrate the results of Example 8.

FIG. 18 indicates that four days after challenge with either Chlamydia trachomatis serovar K or J, mice immunised with Ct-089, Ct-858 and Ct-875 from Chlamydia trachomatis serovar E show lower levels of shedding compared to the adjuvant control. Furthermore, levels of shedding are comparable to those achieved with immunisation by UVEB.

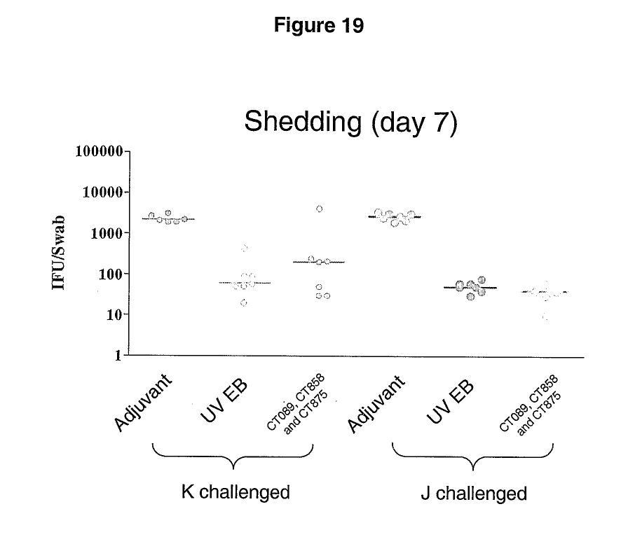

Seven days after challenge mice receiving the adjuvant control show higher levels of shedding than those immunised with UVEB or with Ct-089, Ct-858 and Ct-875 (see FIG. 19). Both UVEB and Ct-089, Ct-858 and Ct-875 immunised mice show very low levels of shedding.

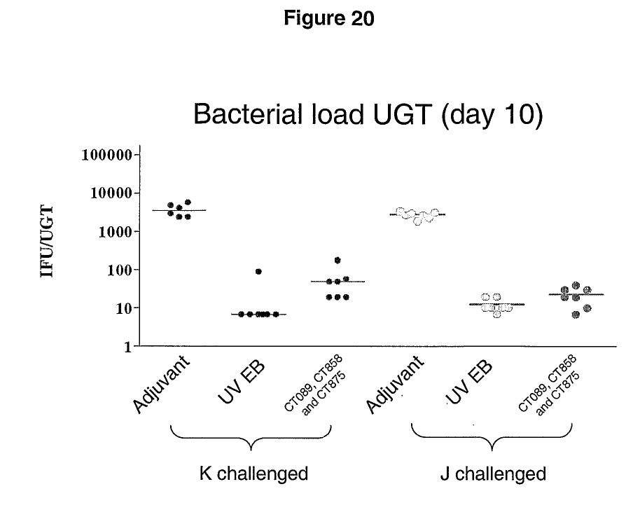

FIG. 20 shows that ten days after challenge with either Chlamydia trachomatis serovar K or J the UGT of mice receiving the adjuvant control is highly colonised by bacteria. Both UVEB and Ct-089, Ct-858 and Ct-875 immunised mice show low levels of UGT colonisation.

Statistical analysis indicates that treatment using the Ct-089, Ct-858 and Ct-875 antigens from Chlamydia trachomatis serovar E results in significant protection in mice challenged with serovar J when compared to the negative control (adjuvant only) with p<0.01. No significant difference is seen between antigen treatment and UVEB treatment (p>0.05).

Treatment using the Ct-089, Ct-858 and Ct-875 antigens from Chlamydia trachomatis serovar E results in significant protection in mice challenged with serovar K on Day 7 and Day 10 when compared to the negative control (adjuvant only) with p<0.05 and p<0.01 respectively. No significant difference is seen between antigen treatment and UVEB treatment (p>0.05).

### Conclusions

The experiments performed in Example 8 confirm that the three proteins, Ct-089, Ct-858 and Ct-875 from Chlamydia trachomatis serovar E are capable of eliciting a substantial protective immune response, which has been shown to be statistically significant. The protection elicited is one which provides general protection against challenge by other serovars.

The level of protection afforded by the vaccine formulation containing Ct-089, Ct-858 and Ct-875 was found to be comparable to the use of UVEB, although clearly the use of purified proteins is desirable in light of the risks involved with the use of the whole elementary bodies.

### Example 9

### Response of Seropositive Individuals to Stimulation by Chlamydial Antigens

The response of seropositive subjects to a number of Chlamydial antigens was examined to investigate which Chlamydia trachomatis antigens may be of particular importance in the normal immune response of humans to Chlamydia trachomatis infection.

### Method

Three subject groups from different locations were involved in the study:
(i) 25 pregnant women who were IgG positive for Chlamydia trachomatis (referred to as BR)
(ii) 19 women who were IgG or PCR positive for Chlamydia trachomatis (referred to as AN)
(iii) 16 individuals of mixed sex (referred to as CR)—
   (a) seven patients who were treated for genital Chlamydia trachomatis infection, identified by Ligase chain reaction and serum titer
   (b) nine non-shedding donors with no history of chlamydial disease, identified by T-cell responses to chlamydial antigens and were not shedding at the time of recruitment to the study. Chlamydia trachomatis IFN-gamma and Chlamydia pneumoniae IFN-gamma response was determined by stimulating 0.3×10⁶ PBMC with 1 ug/ml of the respective chlamydial elementary body. Supernatants were taken after 72 h and analysed by IFN-gamma specific ELISA.

Serology was performed using IgG and IgM complement binding tests supplied by Virion-Serion for subjects in the BR and group, and subjects referred as AN were screened using either the same test or using PCR-techniques (Cobas Amplicor®) on urine samples.

An in vitro assay was performed to evaluate the subjects' T-cell response to various Chlamydia trachomatis antigens and combinations thereof. Peripheral blood mononuclear cell (PBMC) samples were obtained from heparinised whole blood by Ficoll-Hypaque density gradient centrifugation following standard procedures. The cells are washed and cryopreserved in liquid nitrogen until testing (for further details see Lalvani A, Moris P et al. J. Infect. Dis. 1999 180:1656-1664).

For subjects in the CR group, the specific immune response to each Chlamydia trachomatis antigen was characterised by performing lymphocyte proliferation analysis using the tritiated thymidine. This technique assessed the cellular expansion upon in vitro stimulation to an antigen. In practice, cell proliferation is determined by estimating incorporation of tritiated-thymidine into DNA, a process closely related to underlying changes in cell number.

For subjects in the AN and BR groups, the specific immune response to each Chlamydia trachomatis antigen was characterised by performing lymphocyte proliferation analysis using the succinimidyl ester of carboxyfluorsecein diacetate (CFSE). CFSE spontaneously and irreversibly couples to both intracellular and cell surface proteins by reaction with lysine side chains and other available amine groups. When lymphocyte cells divide, CFSE labeling is distributed equally between the daughter cells, which are therefore half as fluorescent as the parents. As a result, halving of cellular fluorescence intensity marks each successive generation in a population of proliferating cells and is readily followed by flow cytometry (for further details see Hodgkins, P D et al J. Exp. Med. 1996 184:277-281).

Practically, after thawing, PMBC were washed and stained with CFSE before being cultivated (2×10⁵ cells) for 72 hrs with 10 ug/ml of antigen in culture media (RPMI-1640 supplemented with glutamine, non essential amino acid, pyruvate and heat inactivated human AB serum). Cells were then harvested and their phenotype was characterized using surface staining to identify memory CD8 and CD4+ T-Cells. Subsequently, flow cytometry analysis indicated the extent of lymphocyte proliferation in response to each antigen (proportion of cells with decreased CFSE intensity upon in vitro stimulation).

### Results

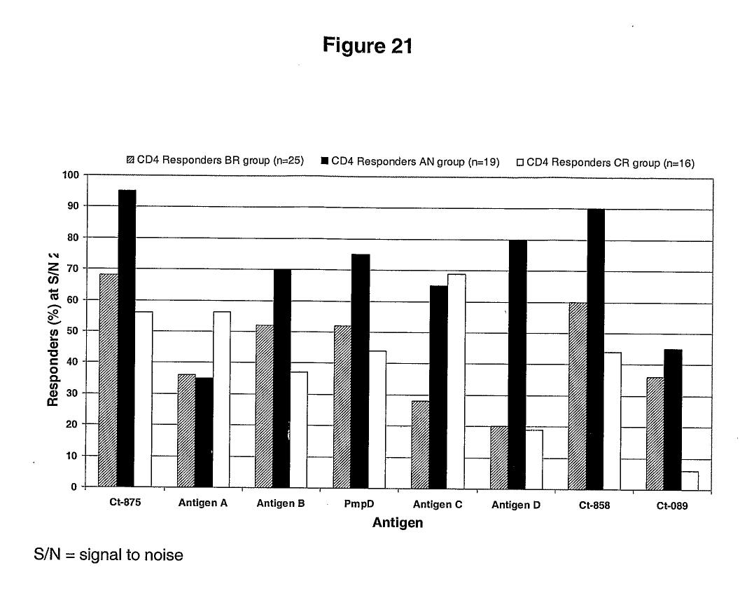

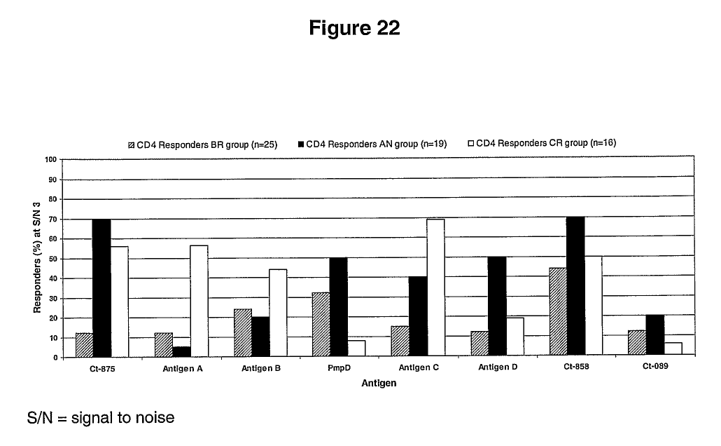

FIG. 21 shows the CD4 response of test subjects to specific antigens, responders in this case from BR and AN groups are defined has having a signal to noise ratio of at least 2:1. Responders in the CR group are defined as those demonstrating a proliferative S/N>10 and IFN-gamma response of 500 ug/ml from T-cells generated from the donor subjects. The proportion of responders can be seen to vary depending upon both the particular antigen being tested and subject group. The AN group generally shows the greatest number of responders (in five out of eight cases), with the CR group generally showing the fewest number of responders (in six out of eight cases) but the specific cellular response was evaluated in another technical setting. Subjects in the BR and AN groups both show their greatest response to the Ct-875 antigen. FIG. 22 shows the CD4 response plotted at a signal to noise ratio of at least 3:1 for BR and AN groups.

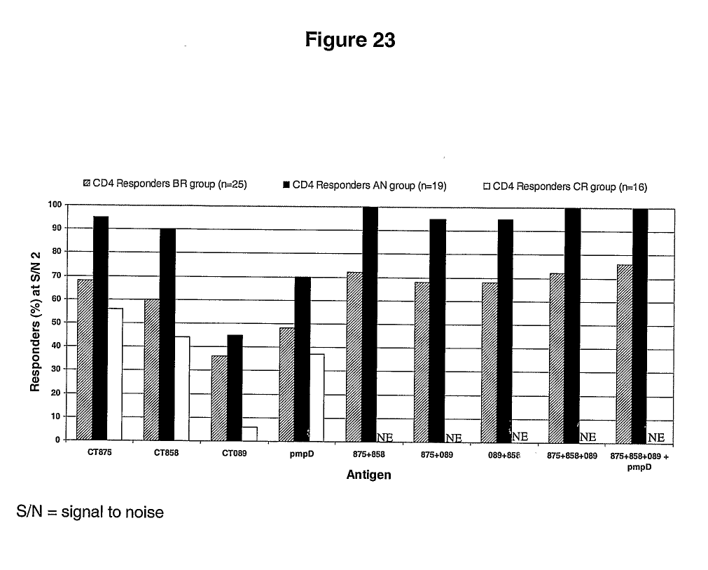

FIG. 23 shows the CD4 response of test subjects to certain antigens, and also to combinations of those antigens (response to the combinations was not examined for the CR subject group, indicated by the notation NE). Compared to the response observed for individual antigens, the combination of Ct-875+Ct-858 or Ct-858+Ct-089 result in a higher proportion of responders in both subject groups. It may be noted that the combination of Ct-875+Ct-089 does not result in any improvement in the number of responders compared to Ct-875 alone, and neither does the combination of Ct-875+Ct-858+Ct-089 result in any improvement in the number of responders compared to the combination of Ct-875+Ct-858. The four antigen combination of Ct-875+Ct-858+Ct-089+PmpD results in the greatest number of responders in the BR subject group (100% response already being observed in the AN group for both the Ct-875+Ct-858 and Ct-875+Ct-858+Ct-089 combinations).

### Conclusions

Although the frequency of responders was not consistent between the three subject groups, possibly as a result of the sample size or population differences, Ct-858 and Ct-875 were well recognized for all three groups.

In the human seropositive subjects tested, the optimal response for a two antigen combination is provided by Ct-875+Ct-858. Ct-089 only seems to result in improved response where Ct-875 is not already present, although Ct-089 may have benefit over and above Ct-875+Ct-858 in other population groups. The greatest response was observed for the four antigen combination of Ct-875+Ct-858+Ct-089+PmpD.

In light of the results of Example 9, it can reasonably be expected that the antigen combinations of the present invention, whether in the form of whole proteins, immunogenic fragments thereof or polynucleotides encoding either of these, will be of particular value in human Chlamydial vaccines and in related diagnostic methods.

1. A composition comprising a combination of two or more Chlamydia proteins or immunogenic fragments thereof or a polynucleotide or polynucleotides encoding them, said two or more proteins or immunogenic fragments selected from Swib, Momp, Ct-858, Ct-875, Ct-622, Ct-089, passenger domain of PmpG (PmpGpd) and passenger domain of PmpD (PmpDpd).

2. A composition according to claim 1 which comprises a Chlamydia Ct-089 protein or an immunogenic fragment thereof, and a Chlamydia Ct-858 protein or an immunogenic fragment thereof, or polynucleotides encoding them.

3. (canceled)

4. A composition according to claim 2, further comprising a Chlamydia Ct-875 protein or an immunogenic fragment thereof or polynucleotide encoding them.

5. A composition according to claim 4, further comprising a Chlamydia PmpDpd protein or an immunogenic fragment thereof or polynucleotide encoding them.

6. (canceled)

7. (canceled)

8. (canceled)

9. (canceled)

10. A composition according to claim 1 further comprising a pharmaceutically acceptable carrier.

11. A composition according to claim 1 further comprising an adjuvant.

12. A composition according to claim 11 wherein the adjuvant is a preferential stimulator of a Th1 response.

13. (canceled)

14. (canceled)

15. (canceled)

16. A composition according to claim 1 wherein two or more of the proteins or immunogenic fragments are linked to form a fusion protein, or the polynucleotide or polynucleotides encoding the protein or immunogenic fragments encodes a fusion of two or more of the proteins or immunogenic fragments.

17. A composition comprising one of the following combinations of Chlamydia polypeptides or immunogenic fragments thereof or polynucleotides encoding them:
1. Momp, PmpDpd, Ct-858, Ct-089, Swib
1′. PmpDpd, Ct-858, Ct-089, Swib
2. Momp, PmpDpd, Ct-858, Ct-622, Ct-089, Swib
3. Momp, PmpDpd, Ct-858, PmpGpd, Ct-622, Ct-089
4. Ct-858, Ct-875, Ct-622, Ct-089
5. Ct-858, Ct-875, Ct-089
5′. PmpDpd, Ct-858, Ct-875, Ct-089
6. Momp, PmpD, Ct-858, PmpGpd, Ct-089
1a. All five of: Momp, PmpDpd, Ct-858, PmpGpd and Ct-089
1′a. Three out of: PmpDpd, Ct-858, Ct-089, Swib
2a. Five out of: Momp, PmpDpd, Ct-858, Ct-622, Ct-089 and Swib
3a. Five out of: Momp, PmpDpd, Ct-858, PmpGpd, Ct-622 and Ct-089
4a. Three out of: Ct-858, Ct-875, Ct-622 and Ct-089
5a. Two out of: Ct-858, Ct-875 and Ct-089
5′a. Three out of: PmpDpd, Ct-858, Ct-875, Ct-089
6a. Four out of: Momp, PmpD, Ct-858, PmpGpd and Ct-089
provided that in all of compositions 1a to 6a, Ct-089 and Ct-858 proteins or immunogenic derivatives or polynucleotides encoding these are present.

18. (canceled)

19. A composition comprising one of the following combinations of Chlamydia polypeptides or immunogenic fragments thereof or polynucleotides encoding them
1b. Momp, PmpDpd, Ct-858, Ct-875, Swib, Ct-089
1b′. PmpDpd, Ct-858, Ct-875, Swib, Ct-089
2b. Momp, PmpDpd, Ct-858, Ct-622, Ct-875, Swib, Ct-089
3b. Momp, PmpDpd, Ct-858, PmpGpd, Ct-622, Ct-875, Ct-089
4b. Ct-858, Ct-875
5b. Momp, Ct-858, Ct-875, Ct-089
5b′. Momp, Ct-858, Ct-875
6b. Momp, PmpD, Ct-858, PmpGpd, Ct-875, Ct-089
1c. Five out of: Swib Momp, PmpDpd, Ct-858, PmpGpd and Ct-875
1c′. Three out of: PmpDpd, Ct-858, Ct-0875, Swib
2c. Five out of: Momp, PmpDpd, Ct-858, Ct-622, Ct-875 and Swib
3c. Five out of: Momp, PmpDpd, Ct-858, PmpGpd, Ct-622 and Ct-875
4c. Three out of: Ct-858, Ct-875, Ct-622 and Ct-089
5c′. Three out of: PmpDpd, Ct-858, Ct-875, Ct-089
6c. Four out of: Momp, PmpD, Ct-858, PmpGpd and Ct-875
provided that in all of compositions 1b to 6c, Ct-875 and Ct-858 proteins or immunogenic derivatives or polynucleotides encoding these are present.

20. A composition according to claim 1, wherein the Swib, Momp, Ct-858, Ct-875, Ct-622, Ct-089, passenger domain of PmpG (PmpGpd) and passenger domain of PmpD (PmpDpd) or immunogenic fragments thereof or polynucleotides, when present in the composition, are:
i. Ct-089—a polypeptide having at least 95% homology to the polypeptide of SEQ ID NO: 16 (Ct-089 from serovar E) or an immunogenic fragment thereof, or a polynucleotide encoding these;
ii. Ct-858—a polypeptide having at least 95% homology to the polypeptide of SEQ ID NO: 6 (Ct-858 from serovar E) or an immunogenic fragment thereof, or a polynucleotide encoding these;
iii. Ct-875—a polypeptide having at least 95% homology to the polypeptide of SEQ ID NO: 8 (Ct-858 from serovar E) or an immunogenic fragment thereof, or a polynucleotide encoding these;
iv. PmpD passenger domain—a polypeptide having at least 95% homology to the polypeptide of SEQ ID NO: 14 (PmpD from serovar LII) or an immunogenic fragment thereof, or a polynucleotide encoding these;
v. Swib—a polypeptide having at least 95% homology to the polypeptide of SEQ ID NO: 2 (Swib from serovar LII) or an immunogenic fragment thereof, or a polynucleotide encoding these;
vi. Momp—a polypeptide having at least 95% homology to the polypeptide of SEQ ID NO: 4 (Momp from serovar F) or an immunogenic fragment thereof, or a polynucleotide encoding these;
vii. Ct-622—a polypeptide having at least 95% homology to the polypeptide of SEQ ID NO: 10 (Ct-622 from serovar E) or an immunogenic fragment thereof, or a polynucleotide encoding these;
viii. PmpG passenger domain—a polypeptide having at least 95% homology to the polypeptide of SEQ ID NO: 12 (PmpG from serovar LII) or an immunogenic fragment thereof, or a polynucleotide encoding these.

21. A composition according to claim 1, wherein all the Chlamydia proteins or immunogenic fragments thereof or a polynucleotide or polynucleotides encoding them in the composition are from Chlamydia trachomatis.

22. (canceled)

23. (canceled)

24. (canceled)

25. A method for the treatment or prevention of Chlamydial infection comprising the administration of a composition according to claim 1.

26. The method according to claim 25, wherein the Chlamydial infection is Chlamydia trachomatis infection.

27. (canceled)

28. (canceled)

29. A method for the treatment or prevention of Chlamydial infection by a second Chlamydia trachomatis serovar, comprising the administration of a vaccine comprising one or more Chlamydial proteins, immunogenic fragments thereof or polynucleotides encoding them, selected from the list consisting of Ct-089, Ct-858 and Ct-875, and which are derived from a first Chlamydia trachomatis serovar.

30. (canceled)

31. The method according to claim 29, wherein the vaccine comprises two proteins, immunogenic fragments thereof or polynucleotides encoding them, selected from the group consisting of Ct-089, Ct-858 and Ct-875.

32. (canceled)

33. (canceled)

34. (canceled)

35. The method according to claim 29, wherein the vaccine comprises Ct-089, Ct-858 and Ct-875, immunogenic fragments thereof or polynucleotides encoding them.

36. The method according to claim 29, wherein the first Chlamydia trachomatis serovar is selected from the list consisting of Chlamydia trachomatis serovars A, B, Ba, C, D, Da, E, F, G, H, I, Ia, J, Ja, K, L1, L2 and L3.

37-44. (canceled)

45. The method according claim 29, wherein the first and second Chlamydia trachomatis serovars are Chlamydia trachomatis serovars which are associated with the same disease state.

46. The method according claim 29, wherein the first and second Chlamydia trachomatis serovars are Chlamydia trachomatis serovars which are associated with different disease states.

47. The method according claim 29, wherein the vaccine comprises one or more additional antigens.

48. (canceled)

49. (canceled)

50. The method according claim 29, wherein the vaccine further comprises an adjuvant.

51. (canceled)

52. (canceled)

53. (canceled)

54. (canceled)

55. A method for determining prior Chlamydial infection in an individual comprising:
(i) obtaining a sample from the individual;
(ii) contacting said sample with a combination of two or more Chlamydia proteins or immunogenic fragments thereof or a polynucleotide or polynucleotides encoding them, said two or more proteins or immunogenic fragments selected from Swib, Momp, Ct-858, Ct-875, Ct-622, Ct-089, passenger domain of PmpG (PmpGpd) and passenger domain of PmpD (PmpDpd);
(iii) quantifying the sample response.

56. The method according to claim 55, wherein the combination of Chlamydia proteins or immunogenic fragments thereof or a polynucleotide or polynucleotides encoding them comprises at least one protein, immunogenic fragment thereof or polynucleotide encoding them, selected from the list consisting of Ct-089, Ct-858 and Ct-875.

57-68. (canceled)

69. A composition according to claim 2 further comprising Momp, Swib, and PmpD or immunogenic fragments thereof or polynucleotides encoding them.
